(19) United States
(12) Patent Application Publication
(10) Pub. No.: US 2023/0312747 A1
(43) Pub. Date: Oct. 5, 2023

(54) Compositions and Methods for Treating Cancer

(71) Applicant: Seagen Inc., Bothell, WA (US)

(72) Inventors: Jillian M. Prendergast, Maynar, MA (US); David A. Eavarone, North Quincy, MA (US); Patricia Rao, Acton, MA (US)

(73) Assignee: Seagen Inc., Bothell, WA (US)

(21) Appl. No.: 18/062,265

(22) Filed: Dec. 6, 2022

Related U.S. Application Data
(63) Continuation of application No. 16/462,084, filed on May 17, 2019, now abandoned.
(60) Provisional application No. 62/563,715, filed on Sep. 27, 2017.
(60) Provisional application No. 62/486,817, filed on Apr. 18, 2017.
(60) Provisional application No. 62/480,096, filed on Mar. 31, 2017.
(60) Provisional application No. 62/443,936, filed on Jan. 9, 2017.
(60) Provisional application No. 62/423,597, filed on Nov. 17, 2016.

(51) Int. Cl. C07K 16/30; A61K 47/68; A61P 35/00; C07K 16/28; G01N 33/574

(52) U.S. Cl. CPC C07K 16/3092; A61K 47/6851; A61K 47/6803; A61P 35/00; C07K 16/2896; G01N 33/57492; A61K 2039/505

(57) ABSTRACT

Methods of detecting, characterizing, and targeting myeloid-derived suppressor cells are disclosed. Some methods include the use of glycan-interacting antibodies, such as anti-STn antibodies. Methods of identifying subjects for treatment with glycan-interacting antibodies are included.

### CROSS REFERENCE TO RELATED APPLICATIONS

This application claims priority to U.S. Provisional Application No. 62/423,597 filed on Nov. 17, 2016 entitled Compositions and Methods for Treating Cancer; U.S. Provisional Application No. 62/443,936 filed on Jan. 9, 2017 entitled Compositions and Methods for Treating Cancer; U.S. Provisional Application No. 62/480,096 filed on Mar. 31, 2017 entitled Compositions and Methods for Treating Cancer; U.S. Provisional Application No. 62/486,817 filed on Apr. 18, 2017 entitled Compositions and Methods for Treating Cancer; and U.S. Provisional Application No. 62/563,715 filed on Sep. 27, 2017 entitled Compositions and Methods for Treating Cancer, the contents of each of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 17, 2017, is named 2033_1025PCT_SL.txt and is 6,905 bytes in size.

### BACKGROUND

Aberrant glycosylation accompanies some of the other mutations commonly observed in carcinomas. It has been estimated that about 80% of all carcinomas express the truncated glycans, the Tn Antigen and the sialylated form, Sialyl Tn (STn). With few exceptions, Tn and STn are not expressed in normal, healthy tissues. Furthermore, the non-human immunogenic sialic acid, N-glycolylneuraminic acid (Neu5Gc), seems to be differentially expressed in some cancers in the form of Neu5Gc-STn (GcSTn).

Multiple aberrant glycosylation forms have been described in human cancers, identifying specific glycans as a class of cell surface molecules suitable for specific tumor targeting (Cheever, M. A. et al., Clin Cancer Res. 2009 Sep. 1; 15(17):5323-37). For example, various human cancer types show high expression of STn antigen, which is rare in normal human tissues (Karlen, P. et al., Gastroenterology. 1998 December; 11 5(6):1395-404; Ohno, S. et al, Anticancer Res. 2006 November-December; 26(6A):4047-53). In addition, the presence of STn on tumor-associated mucins relates to cancer with poor prognosis and is therewith considered an attractive epitope for cancer detection and targeted therapy (Cao, Y. et al., Virchows Arch. 1997 September; 431(3):159-66; Julien, S. et al., Br J Cancer. 2009 Jun. 2; 100(11):1746-54; Itzkowitz, S. H. et al., Cancer. 1990 Nov. 1; 66(9):1960-6; Motoo, Y. et al., Oncology. 1991; 48(4):321-6; Kobayashi, H. et al., J Clin Oncol. 1992 January; 10(1):95-101). Although STn is highly expressed in malignant tissues, low levels are also found on healthy human cells (Jass, J. R. et al., J Pathol. 1995 June; 176(2):143-9; Kirkeby, S. et al., Arch Oral Biol. 2010 November; 55(11):830-41). STn alone has attracted attention as a target for cancer detection and therapy (Cheever, M. A. et al., Clin Cancer Res. 2009 Sep. 1; 15(17):5323-37).

There remains a need in the art for therapeutic antibodies capable of binding glycans, including glycans associated with disease and diseased cells and tissues. Further, there remains a need for methods of using these antibodies to target diseased cells and tissues. The present disclosure meets these needs by providing related compounds and methods.

### SUMMARY OF THE INVENTION

In some embodiments, the present disclosure provides a method of targeting one or more Myeloid-Derived Suppressor Cells (MDSCs) by contacting a subject or subject sample with a glycan-interacting antibody. The subject sample may include at least one MDSC. The glycan-interacting antibody may bind to STn. The glycan-interacting antibody may be an IgG antibody. The glycan-interacting antibody may include a heavy chain variable domain (VH) with an amino acid sequence with at least 70% sequence identity to the amino acid sequence of SEQ ID NO: 1. The glycan-interacting antibody may include a light chain variable domain (VL) with an amino acid sequence with at least 70% sequence identity to the amino acid sequence of SEQ ID NO: 2. Targeted MDSCs may be granulocytic lineage cells, monocytic lineage cells, or immature lineage cells. The method of targeting may preferentially target granulocytic lineage cells.

In some embodiments, the present disclosure provides a method of increasing antitumor cell activity that includes administering glycan-interacting antibodies. The antitumor cell activity may be T cell activity.

In some embodiments, the present disclosure provides a method of treating a subject that includes providing an anti-STn antibody to the subject. The subject may have at least one tumor. The anti-STn antibody may have a heavy chain variable domain (VH) with an amino acid sequence with at least 70% sequence identity to the amino acid sequence of SEQ ID NO: 1. The glycan-interacting antibody may include a light chain variable domain (VL) with an amino acid sequence with at least 70% sequence identity to the amino acid sequence of SEQ ID NO: 2.

The anti-STn antibody may be an IgG antibody. The anti-STn antibody may include an antibody-drug conjugate (ADC). The ADC may include at least one conjugate selected from one or more of an auristatin, a maytansine, a tubulysin, a vinca alkaloid, a pyrrolobenzodiazepine dimer, a camptothecin, a duocarmycin, an amanitin, a phosphoinositide 3-kinase (PI3K) inhibitor, and a mitogen-activated protein kinase (MEK) inhibitor. The ADC may include one or more polymer, wherein the one or more polymer connects the anti-STn antibody and the at least one conjugate. The one or more polymer may include one or more of a poly(ethylene glycol) (PEG), a poly(N-(2-hydroxypropyl)methacrylamide) (polyHPMA), a poly(α-amino acid), a carbohydrate polymer, a glycopolysaccharide, a glycolipid, a glycoconjugate, a polyglycerol, a polyvinyl alcohol, a poly(acrylic acid), a polyketal, and a polyacetal. The one or more polymer may include a poly(1-hydroxymethylethylene hydroxymethylformal) (PHF).

Administering the anti-STn antibody may be carried out in combination with at least one other therapeutic treatment. The other therapeutic treatment may include one or more immune checkpoint inhibitor. The immune checkpoint inhibitor may be a programmed death-1 (PD-1) inhibitor, or a T-lymphocyte antigen 4 (CTLA-4) inhibitor. The at least one tumor may be one or more of a colon tumor, a bladder tumor, an endometrial tumor, a gastric tumor, a kidney tumor, an ovarian tumor, and a pancreatic tumor. The tumor may be a metastatic tumor.

In some embodiments, the present disclosure provides a method of identifying a subject for anti-STn antibody treatment that includes obtaining a sample from the subject; identifying one or more MDSC in the sample; and evaluating the one or more MDSCs for STn expression. The sample may be a tumor sample. The sample may be a fluid sample. The fluid sample may be blood, plasma, lymph, saliva, or interstitial fluid. The fluid sample may be peripheral blood. The one or more MDSC may be a granulocytic MDSC, a monocytic MDSC, and/or an immature MDSC. Identifying the one or more MDSC in the sample may be carried out using at least one of anti-CD33 antibody, anti-HLA-DR antibody, anti-CD45 antibody, anti-CD11b antibody, anti-CD14 antibody, and anti-CD15 antibody. Identifying the one or more MDSC and/or evaluating STn expression by the one or more MDSC may be carried out using flow cytometry.

Methods of the present disclosure include a method of reducing MDSC levels in at least one tissue and/or fluid from a subject by administering an anti-STn antibody to the subject. The levels of granulocytic MDSCs and/or monocytic MDSCs may be reduced. The granulocytic MDSCs and/or monocytic MDSCs may express STn. The tissue may be a tumor tissue or spleen tissue. The percentage of tumor cells expressing STn may be reduced by the anti-STn antibody. The anti-STn antibody may be SIA01. The anti-STn antibody may be an IgG antibody. The anti-STn antibody may be an antibody-drug conjugate. The antibody-drug conjugate may include one or more of an auristatin, a maytansine, a tubulysin, a vinca alkaloid, a pyrrolobenzodiazepine dimer, a camptothecin, a duocarmycin, an amanitin, a PI3K inhibitor, a nucleotide analog, and a MEK inhibitor. Tumor tissues may be from one or more of a bladder tumor, a colon tumor, an endometrial tumor, a gastric tumor, a kidney tumor, an ovarian tumor, and a pancreatic tumor.

### BRIEF DESCRIPTION OF THE FIGURES

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1A is a schematic depicting α2,6-sialylated N-acetylgalactosamine (STn) with the largest ellipse indicating the specific region of STn recognized by Group 1 antibodies.

FIG. 1B is a schematic depicting α2,6-sialylated N-acetylgalactosamine (STn) with the largest ellipse indicating the specific region of STn recognized by Group 2 antibodies.

FIG. 1C is a schematic depicting α2,6-sialylated N-acetylgalactosamine (STn) with the largest ellipse indicating the specific region of STn recognized by Group 3 antibodies.

FIG. 1D is a schematic depicting α2,6-sialylated N-acetylgalactosamine (STn) with the largest ellipse indicating the specific region of STn recognized by Group 4 antibodies.

FIG. 2 is a graph showing the percentage of patient-derived tumor cells expressing STn and CD133 markers.

FIG. 3 is a graph showing the percentage of STn expression in different Myeloid-Derived Suppressor Cell (MDSC) lineages obtained from patient samples.

FIG. 4 is a graph showing the correlation between tumor STn expression level and MDSC STn expression level in a set of patient samples.

FIG. 5 is a graph showing the percentage of STn expression in different MDSC lineages from a human tumor sample and matched blood sample.

FIG. 6A is a graph showing the percentage of STn-positive tumor cells from various patient tumor samples analyzed.

FIG. 6B is a graph showing the percentage of STn-positive MDSCs from MDSC subpopulations isolated from various patient tumor samples analyzed.

FIG. 6C is a graph showing the percentage of STn-positive PMN-MDSCs detected in various patient tumor samples analyzed.

FIG. 6D is a graph showing the correlation between STn expression in PMN-MDSCs and tumor cells analyzed from various patient tumor samples.

FIG. 7A is a graph showing changes in tumor volume over time in mice carrying xenograft tumors from wild-type MDA-MB-231 cells or MDA-MB-231 cells expressing STn (MDA-MB-231 STn+).

FIG. 7B is a graph showing terminal tumor volumes in mice carrying xenograft tumors from wild-type MDA-MB-231 cells or MDA-MB-231 STn+ cells.

FIG. 8 is a graph showing the percentage of tumor cells expressing STn in terminal tumor samples from mice carrying xenograft tumors from wild-type MDA-MB-231 cells or MDA-MB-231 STn+ cells.

FIG. 9 is a graph showing the percentage of cells expressing STn in monocytic MDSC (mMDSC) subpopulations in terminal tumor samples from mice carrying xenograft tumors from wild-type MDA-MB-231 cells or MDA-MB-231 STn+ cells.

FIG. 10 is a graph showing the percentage of cells expressing STn in polymorphonuclear MDSC (PMN-MDSC) subpopulations in terminal tumor samples from mice carrying xenograft tumors from wild-type MDA-MB-231 cells or MDA-MB-231 STn+cells.

FIG. 11 is a graph showing changes in OVCAR3 xenograft tumor volume over time in mice treated with MMAE conjugated SIA01 (SIA01-ADC) or MMAE conjugated IgG isotype control antibody.

FIG. 12 is a graph showing the percentage of MDSCs expressing STn in OVCAR3 xenograft tumors from mice treated with SIA01-ADC or isotype control antibody (MMAE conjugated IgG).

FIG. 13 is a graph showing the percentage of MDSCs in spleen samples from mice carrying OVCAR3 xenograft tumors over the course of treatment with SIA01-ADC or isotype control antibody (MMAE conjugated IgG).

FIG. 14 is a set of graphs showing the percentage of STn+ mMDSCs (left) and percentage of STn+ PMN-MDSCs (right) in spleen samples obtained from mice carrying OVCAR3 xenograft tumors after four weeks of treatment with SIA01-ADC or isotype control antibody (MMAE conjugated IgG).

### DETAILED DESCRIPTION

### Introduction

According to the present disclosure, glycan-interacting antibodies are provided. Glycan-interacting antibodies are antibodies which may be specific for or which may interact with epitopes that include glycans. Such glycans may include α2,6-sialylated N-acetylgalactosamine (STn). In nature, STn may be sialylated with N-acetylneuraminic acid (Neu5Ac) or N-glycolylneuraminic acid (Neu5Gc). Glycan-interacting antibodies according to the present disclosure may be directed to glycans having either STn (pan-STn antibodies), glycans having STn with Neu5Ac specifically (AcSTn), or glycans having STn with Neu5Gc specifically (GcSTn). In some embodiments, glycan-interacting antibodies of the present disclosure target cancer-related glycan antigens, such as STn.

In some embodiments, the present disclosure provides anti-glycan antibodies that target Myeloid-Derived Suppressor Cells (MDSCs). MDSCs are a heterogeneous population of myeloid cell progenitors and precursors of granulocytes, macrophages and dendritic cells (Gabrilovich et al., Nat Rev Immunol., 2009 March; 9(3): 162-174). Under normal conditions, immature myeloid cells (IMCs) that are produced in the bone marrow, differentiate into mature granulocytes, macrophages or dendritic cells. During pathological conditions, such as cancer and autoimmune diseases, there is an induction of IMC proliferation accompanied by partially blocked differentiation. The outcome is the accumulation of MDSCs in the blood, bone marrow, and secondary lymphoid organs (e.g., lymph nodes and spleen) or tissues, including tumors (Gabrilovich et al., Nat Rev Immunol., 2009 March; 9(3): 162-174; Bronte V, et al., Blood. 2000; 96:3838). Their presence in the tumor microenvironment has been suggested to have a causative role in promoting tumor-associated immune suppression. MDSCs are important and unique due their ability to suppress the cytotoxic function of natural killer (NK) and NKT cells, as well as the T cell-mediated adaptive immune response.

In tumor environments, MDSCs reduce antitumor immunity and support tumor expansion and metastasis by secreting factors that promote tumor angiogenesis and lymphagiogenesis (Murdoch C et al. Nat Rev Cancer. 2008; 8:618-631; Yang L, et al., Cancer Cell. 2004; 6:409-421). MDSCs also produce MMPs that enhance tumor cell invasion. MDSCs can also differentiate into tumor-associated macrophages (TAMs) that have complementary activity. Targeting MDSCs, and inhibiting their activity may promote antitumor immune responses important for the control of tumor cells. MDSCs may be identifiable in tumor samples through detection of CD45 and CD133 co-expression.

Functionally, MDSCs are defined by their capacity to suppress T cell immunity through a variety of mechanisms (Nagaraj & Gabrilovich, Cancer J. 2010; 16(4):348-53). MDSC activation may result in production of reactive oxygen species or inducible nitric oxide synthase which can result in complete loss of the effector T cell response (Gabrilovich & Nagaraj, Nat Rev Immunol. 2009; 9(3):162-74). In animal tumor models MDSCs can sterically block major histocompatibility complex (MHC)-bound peptides from being presented to T cells via the TCR-CD8 complex through tyrosine nitration. MDSCs can inhibit T cell function indirectly through signals that promote the expansion of regulatory T cells and induce conversion of T cells to a regulatory phenotype (Gabrilovich & Nagaraj, Nat Rev Immunol. 2009; 9(3):162-74). MDSCs can decrease L-selectin expression on T cells eliminating their infiltration into peripheral lymph nodes where dendritic cell (DC)-mediated sensitization to tumor antigens takes place. MDSCs may achieve this function through depletion of nutrients necessary for T cell function such as L-arginine via arginase-1 activity. They may sequester L-cysteine and limit the availability of this lymphocyte activating amino acid. These functions of T cell inhibition can be rapidly reversed by depleting MDSCs or by restoring the missing nutrients (De Veirman et al., Front Oncol. 2014; 4: 349; Marvel & Gabrilovich, J Clin Invest. 2015; 125(9):3356-64). In mouse models, MDSCs infiltrate tumors and display immature elements of monocyte (M-MDSC) and neutrophil (G-MDSC) morphology (Marvel & Gabrilovich, J Clin Invest. 2015; 125(9):3356-64). Murine MDSCs are phenotypically characterized based on dual expression of CD11b and Ly6 antigens. Murine MDSCs can be subdivided into Ly6Glow (monocyte morphology, M-MDSC or “mMDSC”) and Ly6Ghigh cells (polymorphonuclear morphology, PMN-MDSC or G-MDSC). Human MDSCs express the myeloid markers CD11b and CD33. CD14+HLA-DRlow/− and are defined primarily as the monocytic subpopulation, while human granulocytic MDSCs are primarily defined as being CD15+(Youn et al, J Immunol. 2008; 181(8):5791-802).

Glycans and glycoproteins have been linked to MDSC function and provide attractive targets for immunotherapy. The receptor for advanced glycation end products (RAGE) is modified post-translationally by the addition of carboxylated glycans (Koyama, Mol Med. 2007; 13(11-12):625-35). RAGE is present on the membrane of certain carcinomas and one of its ligands, S100A8/A9, is expressed on murine MDSCs (Vernon et al., Oncoimmunology. 2013; 2(5): e24184). S100A8/A9 is induced via STAT3-mediated signaling and appears to be involved in increasing MDSC infiltration in cancer. Inhibition of S100A8/A9 signaling results in inhibition of MDSC function and decreased tumor growth. Interestingly, RAGE and other cell surface glycoproteins may be directly present on MDSCs as well. MDSC expression of S100A8/A9 and other glycoproteins therefore can complete an autocrine loop that leads to enhanced MDSC accumulation (Vernon et al., Oncoimmunology. 2013; 2(5): e24184) and provides a rationale for the clinical use of anti-glycan antibodies. Additionally, a dramatic reduction in the number and function of MDSCs was reported in ganglioside-deficient tumors and not in ganglioside-rich controls (Wondimu et al., Cancer Res. 2014; 74(19): 5449-5457). Notably, transient ganglioside reconstitution of the tumor cell could restore MDSC infiltration, indicating a direct connection between tumor ganglioside production and the recruitment of immunosuppressive MDSCs. MDSC expression of glycan STn, a known tumor-associated antigen and glycan with established roles in immune regulation, is demonstrated herein.

STn, the sialylated version of the carbohydrate Tn antigen, is rarely expressed in normal adult human tissues, but frequently occurs in human cancers such as breast, ovarian, bladder, cervical, colon, and lung cancer. STn demonstrates high tumor specificity and broad tumor expression. Furthermore, the presence of STn in tumors is associated with metastatic disease, poor prognosis, and reduced overall survival. In some embodiments, anti-glycan antibodies targeting MDSCs include anti-STn antibodies. In some embodiments, such antibodies may include any of those described herein. The anti-STn antibodies may be monoclonal. Some anti-STn antibodies targeting MDSCs may be IgG antibodies. Some anti-STn antibodies may be human or humanized. In some embodiments, anti-STn antibodies used to target MDSCs may include a conjugate. The conjugate may include a cytotoxic agent. Cytotoxic agents may include any of those described herein, including, but not limited to monomethyl auristatin E (MMAE) and monomethyl auristatin F (MMAF).

### Definitions

Adjacent: As used herein, the term “adjacent” refers to something that is adjoining, neighboring or next to a given entity. In some embodiments, “adjacent residues” are sugar residues within a glycan chain that are linked to one another. In some embodiments, “adjacent glycans” are glycan chains that next to each other either in direct contact or within close proximity and without another glycan in between the two.

Administered in combination: As used herein, the term “administered in combination” or “combined administration” means that a subject is simultaneously exposed to two or more agents administered at the same time or within an interval of time such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more glycan-interacting antibodies, as described herein, are spaced sufficiently closely together such that a combinatorial (e.g., a synergistic) effect is achieved.

Amino acid: As used herein, the terms “amino acid” and “amino acids” refer to all naturally occurring L-alpha-amino acids as well as non-naturally occurring amino acids. Amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: As used herein, the term “antibody” is used in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also include one or more modifications such as with sugar moieties.

Antibody fragment: As used herein, the term “antibody fragment” refers to a portion of an intact antibody, preferably including an antigen binding region thereof. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Glycan-interacting antibodies may include one or more of these fragments. For the purposes herein, an antibody may include a heavy and light variable domain as well as an Fc region.

Antigen-binding region: As used herein, the term “antigen-binding region” refers to the portion of an antibody, antibody fragment, or related molecule that directly interacts with a target molecule or epitope. Antigen-binding regions typically include a variable domain pair, as in the Fab region of an antibody or as linked together in a scFv.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biomolecule: As used herein, the term “biomolecule” is any natural molecule which is amino acid-based, nucleic acid-based, carbohydrate-based or lipid-based, and the like.

Bispecific antibody: As used herein, the term “bispecific antibody” refers to an antibody capable of binding two different antigens. Such antibodies typically include regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

Branch: As used herein, the term “branch” refers to an entity, moiety or appendage that is linked or extends out from a main entity or source. In some embodiments, a “branch chain” or “branching chain” includes one or more residues (including, but not limited to sugar residues) that extend from a parent chain. As used herein, a “parent chain” is used to refer to a chain of residues (including, but not limited to sugar residues) from which a branching chain is linked. In the case of a glycan with multiple branches, the parent chain may also refer to the source chain from which all such branches are directly or indirectly attached. In the case of a polysaccharide having a chain of hexose residues, parent chain linkages typically occur between carbons 1 and 4 of adjacent residues while branching chains are attached to a parent chain through a linkage between carbon 1 of the branching residue and carbon 3 of the parent residue from which the branch extends. As used herein, the term “branching residue” refers to the residue attached to the parent chain in a branching chain.

Cancer stem cells: As used herein, cancer stem cells (CSCs) refer to a subset of tumor cells that have the ability to self-renew. CSCs may be able to regenerate diverse cell types. In some cases, these cells are difficult or impossible to remove through surgical or chemical treatment of a tumor.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Cyclic or Cyclized: As used herein, the term “cyclic” refers to the presence of a continuous loop. Cyclic molecules need not be circular, only joined to form an unbroken chain of subunits.

Cytidine monophosphate-N-acetylneuraminic acid hydroxylase: As used herein, the term “cytidine monophosphate-N-acetylneuraminic acid hydroxylase” or “CMAH” refers to an enzyme, absent in humans, but present in most other mammals (including, but not limited to mice, pigs and chimpanzees) that catalyzes the formation of N-glycolylneuraminic acid from N-acetylneuraminic acid. The absence of the enzyme in humans is due to a frameshift mutation resulting in the premature termination of the CMAH transcript and the production of a non-functional protein.

Cytotoxic: As used herein, the term “cytotoxic” is used to refer to an agent that kills or causes injurious, toxic, or deadly effects on a cell (e.g., a mammalian cell (e.g., a human cell)), bacterium, virus, fungus, protozoan, parasite, prion, or a combination thereof.

Delivery: As used herein, “delivery” refers to the act or manner of transporting a compound, substance, entity, moiety, cargo or payload to an intended destination.

Delivery Agent: As used herein, “delivery agent” refers to any substance which facilitates, at least in part, the in vivo delivery of a compound, substance, entity, moiety, cargo or payload.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Display library: As used herein, the term “display library” refers to a tool used in scientific discovery to identify biomolecular interactions. Different variations of display libraries exist that include the utilization of bacteriophages, yeast and ribosomes. In each case, proteins within a given library (also referred to herein as “library members”) are linked (physically or through association with a host) to the nucleic acid which encodes the protein. When a target molecule is incubated with the members of a display library, any library members that bind to the target may be isolated and the sequences encoding the bound protein may be determined through analysis of the linked nucleic acid. In some embodiments, display libraries are “phage display libraries” wherein the display library is made up of bacteriophage viral particles (also referred to herein as “phage particles”) wherein nucleic acids have been incorporated into the phage genome resulting in the production of viral coat proteins that are fused to proteins encoded by the nucleic acids that have been introduced. Such fused proteins are “displayed” on the outer surface of the assembled phage particles where they may interact with a given target.

Distal: As used herein, the term “distal” means situated away from the center or away from a point or region of interest.

Engineered: As used herein, embodiments of the invention are “engineered” when they are designed to have a feature or property, whether structural or chemical, that varies from a starting point, wild type or native molecule. Thus, engineered agents or entities are those whose design and/or production include an act of the hand of man.

Epitope: As used herein, an “epitope” refers to a surface or region on a molecule that is capable of interacting with components of the immune system, including, but not limited to antibodies. In some embodiments, an epitope may include a target site. Epitopes may include a region on an antigen or between two or more antigens that is specifically recognized and bound by a corresponding antibody. Some epitopes may include one or more sugar residues along one or more glycan. Such epitopes may include 1, 2, 3, 4, 5, 6, 7, 8, 9 or at least 10 sugar residues. Epitopes may also include one or more regions of interaction between entities. In some embodiments, epitopes may include a junction between two sugar residues, between a branching chain and a parent chain or between a glycan and a protein.

Ether bond: As used herein, an “ether bond” refers to a chemical bond that includes an oxygen bonded between two carbon atoms. In some embodiments, ether bonds link sugar residues to other entities, including, but not limited to other sugar residues to form a glycan chain. Such bonds are also referred to as “glycosidic bonds” or “glycosidic linkages”. In the context of at least one sugar residue, the terms “link” and/or “linkage” are also used herein when referring to a glycosidic linkage. In some embodiments, linkages may link glycans to other entities, including, but not limited to proteins, lipids, phospholipids and sphingolipids. In some embodiments, sugar residues may be linked to protein, typically forming a link between a sugar residue and an amino acid residue. Such amino acid residues include serine and threonine. In some embodiments, ether bonds link glycans to a glycan array through a carbohydrate linker that participates in bond formation. Glycosidic linkages may differ in their stereochemical properties. In some embodiments, alpha oriented glycosidic linkages (also referred to herein as “alpha linkages”) result in an axial orientation between the bonded oxygen of the ether bond and the cyclohexane ring of the sugar reside. In some embodiments, beta oriented glycosidic linkages (also referred to herein as “beta linkages”) result in an equatorial orientation between the bonded oxygen of the ether bond and the cyclohexane ring of the sugar residue.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” refers to a material or mixture prepared according to a formula and which may include at least one antibody, compound, substance, entity, moiety, cargo or payload and a delivery agent, carrier or excipient.

Functional: As used herein, a “functional” biological molecule is a biological entity with a structure and in a form in which it exhibits a property and/or activity by which it is characterized. As used herein, a “functional group” or “chemical group” refers to a characteristic group of atoms or chemical bonds that are part of a larger molecule. In some embodiments, functional groups may be associated with different molecules, but may participate in similar chemical reactions regardless of the molecule of which they are a part. Common functional groups include, but are not limited to carboxyl groups (—COOH), acetyl groups (—COH), amino groups (—NH₂), methyl groups (—CH₃), sulfate groups (—SO₃H) and acyl groups. In some embodiments, the addition of one or more functional group to a molecule may be conveyed using terms that modify the name of the functional group with the ending “-ylated”, e.g., acetylated, methylated and sulfated.

Glycan: As used herein, the terms “glycan”, “oligosaccharide” and “polysaccharide” are used interchangeably and refer to polymers made up of sugar monomers, typically joined by glycosidic bonds also referred to herein as linkages. In some embodiments, the terms “glycan”, “oligosaccharide” and “polysaccharide” may be used to refer to the carbohydrate portion of a glycoconjugate (e.g., glycoprotein, glycolipid or proteoglycan).

Glycan chain: As used herein, the term “glycan chain” refers to a sugar polymer that includes two or more sugars. In some embodiments, glycan chains are covalently linked to proteins through serine or threonine residues on the protein.

Glycan-rich composition: As used herein, the term “glycan-rich composition” refers to a mixture that includes a large percentage of glycans. In some embodiments, glycans within a glycan-rich composition may make up from about 1% to about 10%, from about 5% to about 15%, from about 20% to about 40%, from about 30% to about 50%, from about 60% to about 80%, from about 70% to about 90% or at least 100% of the total weight of the composition.

Glycosidic bond: As used herein, the term “glycosidic bond” refers to a covalent bond formed between a carbohydrate and another chemical group. In some embodiments, glycosidic bonds are formed between the reducing end of one sugar molecule and the non-reducing end of a second sugar molecule or polysaccharide chain. Such glycosidic bonds are also known as O-glycosidic bonds due to the oxygen (or ether bond) between the joined sugars. In some embodiments, a glycosidic bond between two sugars or between a sugar and a linker may also be referred to as a “linkage”.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kit: As used herein, the term “kit” refers to a set that includes one or more components adapted for a cooperative purpose and instructions for use thereof.

Knockout: As used herein, the term “knockout” refers to an organism wherein an existing gene has been inactivated through a process that typically involves the hand of man. In a knockout organism, a gene that has been inactivated is said to have been “knocked out”. In some embodiments, the knocked-out gene may be inactivated through the insertion of a nucleotide sequence into the gene or through replacement of the gene entirely.

Linker: As used herein, a “linker” refers to a moiety that connects two or more domains, moieties or entities. In one embodiment, a linker may include 10, 11, 12, 13, 14, 15 or more atoms. In a further embodiment, a linker may include a group of atoms, e.g., 10-1,000 atoms. Such atoms or groups thereof may include, but are not limited to, carbon, amino, alkylamino, oxygen, sulfur, sulfoxide, sulfonyl, carbonyl, and imine. In some embodiments, the linker may include an amino acid, peptide, polypeptide or protein. In some embodiments, a moiety bound by a linker may include, but is not limited to an atom, a chemical group, a nucleoside, a nucleotide, a nucleobase, a sugar, a nucleic acid, an amino acid, a peptide, a polypeptide, a protein, a protein complex, a payload (e.g., a therapeutic agent) or a marker (including, but not limited to a chemical, fluorescent, radioactive or bioluminescent marker). The linker can be used for any useful purpose, such as to form multimers or conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bonds include an amido bond which may be cleaved for example by the use of tris (2-carboxyethyl)phosphine (TCEP), or other reducing agents, and/or photolysis, as well as an ester bond which may be cleaved for example by acidic or basic hydrolysis. In some embodiments, a linker is a carbohydrate moiety used to link glycans to a substrate, such as in a glycan array. Such carbohydrate linkers include, but are not limited to —O(CH₂)₂CH₂HN₂ and —O(CH₂)₃NHCOCH₂(OCH₂CH₂)₆NH₂.

mRNA: As used herein, the term “mRNA” refers to messenger RNA produced as a result of gene transcription and processing of the generated transcript. In some embodiments, mRNA that has left the nucleus of the cell may be extracted from a cell or set of cells and analyzed to determine which genes have undergone transcription at a given time or under a given set of circumstances.

Mucin: As used herein, the term “mucin” refers to a family of proteins that are heavily glycosylated. In some embodiments mucins are produced by the submaxillary glands and are found in saliva and mucous.

Negative selection: As used herein, the term “negative selection” refers to the selection of library members from a display library based on their ability to bind entities and/or components of a composition that do not include a target antigen. In some embodiments, negative selection is used prior to positive selection to remove elements that might bind non-specifically to the target.

Off-target: As used herein, “off target” refers to any unintended effect on any one or more target, gene, or cellular transcript.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Peptide: As used herein, “peptide” is a protein or polypeptide which is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than active agents (e.g., as described herein) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, dispensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and ti-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Positive selection: As used herein, the term “positive selection” refers to the selection of a given entity from a group of unique entities. Such entities and groups thereof may be, for example antibodies. In some cases, they may be antibody fragments or antibody fragments expressed in association with an agent capable of expressing such fragments (e.g. library members from a display library). Selection may be based on the ability of selected entities to bind to a desired target or epitope. In some embodiments, positive selection may be used with phage display libraries to identify phage particles expressing scFvs that bind to the desired target. In other embodiments, positive selection may refer to the selection of antibody candidates from among a pool of antibodies. In other cases, entities may be cells, cell lines or clones as in the selection of clones during hybridoma selection. In such cases, positive selection may refer to clonal selection based on one or more features of antibodies (e.g. specificity for one or more desired epitopes) produced by such clones. In some cases, desired epitopes in positive selection methods may include STn (e.g. AcSTn and/or GcSTn).

Conversely, “negative selection,” as used herein, included the same principles and examples described for positive selection, but with the distinguishing characteristic that it is used for removal of undesired entities from a group of unique entities.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Proximal: As used herein, the term “proximal” means situated nearer to the center or to a point or region of interest.

Region of interaction: As used herein, the term “region of interaction” refers to a region along any of two or more entities where such entities interact or overlap. In some embodiments, a region of interaction may include one or more sugar residues along a glycan chain that contacts a second glycan chain. In some embodiments, the glycan chains are branching chains from the same parent chain. In some embodiments, a region of interaction may occur between two glycan chains wherein one chain is a branching chain and the second chain is a parent chain. In the case of glycan chains, regions of interaction may include 1, 2, 3, 4, 5, 6, 7, 8, 9 or at least 10 sugar residues. In some embodiments, regions of interaction may also occur between glycans and proteins or between glycans and lipids.

Residue: As used herein, the term “residue” refers to a monomer associated with or capable of associating with a polymer. In some embodiments, residues include sugar molecules including, but not limited to glucose, galactose, N-acetylglucosamine, N-acetylgalactosamine, sialic acids. In some embodiments, residues include amino acids.

Sample: As used herein, the term “sample” refers to an aliquot or portion taken from a source and/or provided for analysis or processing. In some embodiments, a sample is from a biological source (also referred to herein as a “biological sample”) such as a tissue, cell or component part (e.g. a body fluid, including but not limited to blood, plasma, serum, mucus, lymphatic fluid, synovial fluid, cerebrospinal fluid, saliva, amniotic fluid, amniotic cord blood, urine, vaginal fluid and semen). In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample includes a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Sialyl: As used herein, the prefix “sialyl” as well as the term “sialylated” describe compounds including sialic acid.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Submaxillary glands: As used herein, the term “submaxillary glands” or “submandibular glands” refers to mucous producing glands located beneath the mouth floor. These glands are capable of producing mucins and in some embodiments, may be extracted from mammals as a source of mucin.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Target: As used herein, the term “target” refers to an object or entity to be affected by an action. In some embodiments, targets refer to antigens to be used for the development of antibodies that specifically bind the antigens.

Target screening: As used herein, the term “target screening” refers to the use of a target substance to identify binding partners for that substance.

Target site: As used herein, the term “target site” refers to a region on or within one or more glycans, glycoproteins, biomolecules and/or biostructures on or within a cell, the extracellular space, a tissue, an organ and/or an organism that is recognized by a binding agent or effector molecule (e.g., an antibody). In some embodiments, glycan target sites may reside exclusively on one sugar residue, may be formed by two or more residues, or may include both glycan and non-glycan components. In some embodiments, target sites are formed between two or more glycans or glycoproteins. In some embodiments, target sites are formed between branching chains of the same glycan or between one or more branching chains and a parent chain.

Target cell: As used herein, a “target cell” refers to cells of interest, including a cell intended to be affected by a given action or process. In some embodiments, a cell that interacts with an antibody is referred to as a “target” of that antibody. The target cell may be a cell that is disrupted or killed by an antibody carrying a cytotoxic conjugate. Target cells may be found in vitro, in vivo, in situ or in the tissue or organ of an organism. The organism may be an animal, mammal, or human (e.g., a human patient).

Terminal residue: As used herein, the term “terminal residue” refers to the last residue in a polymeric chain. In some embodiments, terminal residues are sugar residues located at the non-reducing end of a polysaccharide chain.

Therapeutic agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen that includes a plurality of doses. Those skilled in the art will appreciate that in some embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transgenic: As used herein, the term “transgenic” refers to an organism that includes one or more genes incorporated within the organism's genome that are not naturally found in that organism.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Variable region: As used herein, the term “variable region” or “variable domain” refers to specific antibody domains that differ extensively in sequence among antibodies and are used in the binding and specificity of each particular antibody for its particular antigen.

Whole IgG: As used herein, the term “whole IgG” refers to a complete IgG molecule. In some embodiments, whole IgG molecules include regions found naturally in two or more other organisms.

Wild type: As used herein, the term “wild type” refers to an organism that includes a natural genome (free from genes derived from other organisms).

### I. Compounds and Compositions

In some embodiments, the present invention provides compounds as well as compositions that include at least one glycan-interacting antibody. Within a glycan, monosaccharide monomers may all be the same or they may differ. Common monomers include, but are not limited to trioses, tetroses, pentoses, glucose, fructose, galactose, xylose, arabinose, lyxose, allose, altrose, mannose, gulose, iodose, ribose, mannoheptulose, sedoheptulose and talose. Amino sugars may also be monomers within a glycan. Glycans including such sugars are herein referred to as aminoglycans. Amino sugars, as used herein, are sugar molecules that include an amine group in place of a hydroxyl group, or in some embodiments, a sugar derived from such a sugar. Examples of amino sugars include, but are not limited to glucosamine, galactosamine, N-acetylglucosamine, N-acetylgalactosamine, sialic acids (including, but not limited to, N-acetylneuraminic acid and N-glycolylneuraminic acid) and L-daunosamine.

As used herein the term “glycan-interacting antibody” refers to an antibody that can interact with a glycan moiety. Such antibodies may bind to a glycan moiety alone, to multiple glycan moieties, or to epitopes that include both glycan and non-glycan components. Non-glycan components may include, but are not limited to proteins, protein-associated moieties (such post-translational modifications), cells, and cell-associated molecules/structures. Glycan-interacting antibodies may function to bind to, alter, activate, inhibit, stabilize, degrade and/or modulate a glycan or a glycan-associated molecule or entity. In so doing, glycan-interacting antibodies may function as a therapeutic, whether palliative, prophylactic or as an ongoing treatment composition. In some embodiments, glycan-interacting antibodies may include conjugates or combinations with other molecules. In some embodiments, glycan-interacting antibodies are directed toward glycans having one or more amino sugar. In a further embodiment, one or more amino sugars is a sialic acid. In a further embodiment, one or more sialic acids is N-acetylneuraminic acid and/or N-glycolylneuraminic acid.

### Antibodies

Glycan-interacting antibodies may include entire antibodies or fragments thereof. As used herein, the term “antibody” is used in the broadest sense and embraces various formats including, but not limited to monoclonal antibodies, polyclonal antibodies, multispecific antibodies (e.g., bispecific antibodies formed from at least two intact antibodies), antibody conjugates (including, but not limited to antibody-drug conjugates), antibody variants [including, but not limited to antibody mimetics, chimeric antibodies (e.g. antibodies with amino acid sequences derived from more than one species), and synthetic variants], and antibody fragments, so long as they exhibit a desired biological activity (e.g., binding, activating, inhibiting, stabilizing, degrading, and/or modulating one or more targets). Antibodies are primarily amino-acid based molecules but may include one or more post-translational or synthetic modifications. Post-translational modifications may include glycosylation.

As used herein, the term “antibody fragment” refers to a portion of an intact antibody or fusion-protein thereof, in some cases including at least one antigen binding region. Examples of antibody fragments include Fab, Fab′, F(ab′)₂, Fv fragments, single-chain variable fragments (scFvs); diabodies; tri(a)bodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Glycan-interacting antibodies may include one or more of these fragments and may, for example, be generated through enzymatic digestion of whole antibodies or through recombinant expression.

“Native antibodies” are usually heterotetrameric glycoproteins of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains include hypervariable regions. As used herein, the term “hypervariable region” refers to a region within a variable domain that includes amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody that includes a structure that is complimentary to its target antigen or epitope. Other portions of the variable domain, not interacting with the antigen, are referred to as framework (FW) regions. The antigen-binding site (also known as the antigen combining site or paratope) includes the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia PA. 2012. Ch. 3, p 47-54, the contents of which are herein incorporated by reference in their entirety). Determining residues making up CDRs may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and Al-Lazikani, B. et al., 1997, J. Mol. Biol. 273(4):927-48, the contents of each of which are herein incorporated by reference in their entirety], Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

VH and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs have favored canonical structures with the exception of the CDR-H3, which includes amino acid sequences that may be highly variable in sequence and length between antibodies resulting in a variety of three-dimensional structures in antigen-binding domains (Nikoloudis, D. et al., 2014. PeerJ. 2:e456). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia PA. 2012. Ch. 3, p 47-54, the contents of which are herein incorporated by reference in their entirety).

As used herein, the term “Fv” refers to an antibody fragment that includes the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia PA. 2012. Ch. 3, p 46-47, the contents of which are herein incorporated by reference in their entirety).

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2a, IgG2b, IgG2c, IgG3, IgG4, IgA, and IgA2.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

The term “diabodies” refers to small antibody fragments with two antigen-binding sites, which fragments include a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), the contents of each of which are incorporated herein by reference in their entirety.

The term “intrabody” refers to a form of antibody that is not secreted from a cell in which it is produced, but instead target one or more intracellular protein. Intrabodies may be used to affect a multitude of cellular processes including, but not limited to intracellular trafficking, transcription, translation, metabolic processes, proliferative signaling and cell division. In some embodiments, methods of the present invention may include intrabody-based therapies. In some such embodiments, variable domain sequences and/or CDR sequences disclosed herein may be incorporated into one or more construct for intrabody-based therapy. In some cases, intrabodies of the invention may target one or more glycated intracellular protein or may modulate the interaction between one or more glycated intracellular protein and an alternative protein.

The term “chimeric antigen receptor” or “CAR” as used herein, refers to artificial receptors that are engineered to be expressed on the surface of immune effector cells resulting in specific targeting of such immune effector cells to cells expressing entities that bind with high affinity to the artificial receptors. CARs may be designed to include one or more segments of an antibody, antibody variable domain and/or antibody CDR, such that when such CARs are expressed on immune effector cells, the immune effector cells bind and clear any cells that are recognized by the antibody portions of the CARs. In some cases, CARs are designed to specifically bind cancer cells, leading to immune-regulated clearance of the cancer cells.

The term “monoclonal antibody” as used herein refers to an antibody obtained from a population of substantially homogeneous cells (or clones), i.e., the individual antibodies making up the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

“Humanized” forms of non-human (e.g., murine) antibodies are chimeric antibodies that contain minimal sequences derived from non-human immunoglobulins. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity. Humanized antibodies may include one or more back-mutation that include the reversion of one or more amino acids back to amino acids found in a donor antibody. Conversely, residues from donor antibodies included in humanized antibodies may be mutated to match residues present in human recipient antibodies.

In some embodiments, glycan-interacting antibodies of the present invention may be antibody mimetics. The term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure, sequence and/or function, but including some differences in their amino acid sequence, composition or structure as compared to another antibody or a native antibody.

### Antibody Development

Glycan-interacting antibodies of the present invention are developed to bind antigens such as those described herein. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. In some cases, methods of immunization may be altered based on one or more desired immunization outcomes. As used here, the term “immunization outcome” refers to one or more desired effects of immunization. Examples include high antibody titers and/or increased antibody specificity for a target of interest.

Antigens of the invention may include glycans, glycoconjugates (including, but not limited to glycoproteins and glycolipids), peptides, polypeptides, fusion proteins, or any of the foregoing and may be conjugated or complexed to one or more separate adjuvants or heterologous proteins. In some embodiments, antigens used according to methods of the present invention may include sialylated glycans, such as STn. Antigens having STn may include mucins. Mucins are a family of proteins that are heavily glycosylated. They are a component of many tumors originating from epithelial cells (Ishida, A. et al., 2008. Proteomics. 8: 3342-9, the contents of which are herein incorporated by reference in their entirety). They are highly expressed by submaxillary glands and can be found at high levels in saliva and mucous. Animal-derived submaxillary mucins may be used as antigens to generate anti-STn antibodies in immunogenic hosts. Submaxillary mucin from different species differ in their STn content with regard to AcSTn versus GcSTn forms. Porcine submaxillary mucin (PSM) is particularly rich in GcSTn, which makes up about 90% of total STn. STn from bovine submaxillary mucin (BSM) includes roughly equal percentages of GcSTn and AcSTn. Ovine submaxillary mucin (OSM) is particularly rich in AcSTn, which makes up about 90% of total STn. In some cases, solutions prepared for immunization may be modified to include one or more of PSM, BSM and OSM depending on the desired target of antibodies resulting from such immunization. PSM may be used in immunizations to generate antibodies in immunogenic hosts that are more likely to be specific for GcSTn. PSM is rich in Neu5Gc-containing mucin-type, glycoproteins that are decorated with GcSTn. Among the currently known sources of high Neu5Gc content is red meat; especially submaxillary glands were previously described as a rich source of Neu5Gc due to the high expression of the CMAH enzyme, which catalyzes the reaction to produce the Neu5Gc precursor, CMP-Neu5Ac. In some cases, PSM may be used to prevent a pan-anti-Neu5Gc response and induce a more specific immune response against GcSTn. OSM may be used in immunizations to generate antibodies in immunogenic hosts that are more likely to be specific for AcSTn.

In one embodiment, the present invention provides a glycan-interacting antibody that is GcSTn-specific. The antibody has little cross-reactivity to Neu5Ac-STn or Tn. The antibody can bind GcSTn but has reduced affinity for AcSTn.

In some embodiments, antigens may be subjected to enzymatic digestion prior to immunization to modulate the resulting immune response in immunogenic hosts. In one example, submaxillary mucins may be treated with trypsin or proteinase K enzymes prior to immunization. The activity of such enzymes may help to cleave off and thereby reduce the percentage and variability of non-STn epitopes. Glycan moieties may shield regions of the peptide where they are attached from enzymatic proteolysis and thereby remain intact.

Antibody titers resulting from immunizations may have different antibody levels depending on the type and amount of antigen used in such immunizations. In some cases, certain antigens may be selected for use in immunizations based on the expected titer.

As used herein, an “adjuvant” is a pharmacological or immunological agent that modifies the effect of other agents. Adjuvants according to the present invention include, but are not limited chemical compositions, biomolecules, therapeutics, and/or therapeutic regimens. Adjuvants may include Freund's adjuvant (complete and/or incomplete), immunostimulatory oligonucleotides [e.g. CpG oligodeoxynucleotides (ODNs)], mineral-containing compositions, bacterial ADP-ribosylating toxins, bioadhesives, mucoadhesives, microparticles, lipids, liposomes, muramyl peptides, N-oxidized polyethylene-piperazine derivatives, saponins and/or immune stimulating complexes (ISCOs). In some embodiments, adjuvants may include oil-in-water emulsions (e.g. sub-micron oil-in-water emulsions). Adjuvants according to the present invention may also include any of those disclosed in US Patent Publication No. US20120027813 and/or U.S. Pat. No. 8,506,966, the contents of each of which are herein incorporated by reference in their entirety.

Antibodies of the present invention may be polyclonal or monoclonal or recombinant, produced by methods known in the art or as described in this application. In some embodiments, the antibodies of the present invention may be labeled for purposes of detection with a detectable label known by one of skill in the art. The label can be a radioisotope, fluorescent compound, chemiluminescent compound, enzyme, or enzyme co-factor, or any other labels known in the art. In some aspects, the antibody that binds to a desired antigen is not labeled, but may be detected by binding of a labeled secondary antibody that specifically binds to the primary antibody.

Antibodies of the present invention (e.g., glycan-interacting antibodies) include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), intracellularly made antibodies (i.e., intrabodies), and epitope-binding fragments of any of the above. Antibodies of the present invention (e.g., glycan-interacting antibodies) can be from any animal origin including birds and mammals. Preferably, such antibodies are of human, murine (e.g., mouse and rat), donkey, sheep, rabbit, goat, guinea pig, camel, horse, or chicken origin. The antibodies of the present invention can be monospecific or multispecific (e.g., bispecific, trispecific, or of greater multispecificity). Multispecific antibodies can be specific for different epitopes of a target antigen of the present invention, or can be specific for both a target antigen of the present invention, and a heterologous epitope, such as a heterologous glycan, peptide or solid support material. (See, e.g., WO 93/17715; WO 92/08802; WO 91/00360; WO 92/05793; Tutt, A. et al., Trispecific F(ab)3 derivatives that use cooperative signaling via the TCR CD3 complex and CD2 to activate and redirect resting cytotoxic T cells. J Immunol. 1991 Jul. 1; 147(1):60-9; U.S. Pat. Nos. 4,474,893; 4,714,681; 4,925,648; 5,573,920; 5,601,819; and Kostelny, S. A. et al., Formation of a bispecific antibody by the use of leucine zippers. J Immunol. 1992 Mar. 1; 148(5):1547-53).

Glycan-interacting antibodies of the present disclosure may be prepared using well-established methods known in the art for developing monoclonal antibodies. In one embodiment, the monoclonal antibodies are prepared using hybridoma technology (Kohler, G. et al., Continuous cultures of fused cells secreting antibody of predefined specificity. Nature. 1975 Aug. 7; 256(5517):495-7). For hybridoma formations, first, a mouse, hamster, or other appropriate host animal, is typically immunized with an immunizing agent (e.g., a target antigen of the invention) to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, J. W., Monoclonal Antibodies: Principles and Practice. Academic Press. 1986; 59-1031). Immortalized cell lines are usually transformed mammalian cells, particularly myeloma cells of rodent, rabbit, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, D. et al., A human hybrid myeloma for production of human monoclonal antibodies. J Immunol. 1984 December; 133(6):3001-5; Brodeur, B. et al., Monoclonal Antibody Production Techniques and Applications. Marcel Dekker, Inc., New York. 1987; 33:51-63).

In some embodiments, myeloma cells may be subjected to genetic manipulation. Such manipulation may be carried out using zinc-finger nuclease (ZFN) mutagenesis as described herein. Alternatively, transfection methods known in the art may be used. NS0 myeloma cells or other mouse myeloma cell lines may be used. For example, Sp2/0-Ag14 can be an alternative cell line for hybridoma development.

Transcription Activator-Like Effector Nucleases (TALENs)-induced gene editing provides an alternative gene knock out method. TALENs are artificial restriction enzymes generated by fusing the TAL effector DNA binding domain to a DNA cleavage domain. Similar to ZFNs, TALENs induce double-strand breaks at desired loci that can be repaired by error-prone NHEJ to yield insertions/deletions at the break sites (Wood, A. J. et al., Targeted genome editing across species using ZFNs and TALENs. Science. 2011 Jul. 15; 333(6040):307). Cellectis Bioresearch (Cambridge, MA) provides the service of TALEN design and plasmid construction. The culture medium in which the hybridoma cells are cultured can then be assayed for the presence of monoclonal antibodies. Preferably, the binding specificity (i.e., specific immunoreactivity) of monoclonal antibodies produced by the hybridoma cells is determined by immunoprecipitation or by an in vitro binding assay, such as radioimmunoassay (RIA) or enzyme-linked immunosorbent assay (ELISA). Such techniques and assays are known by those skilled in the art. The binding specificity of the monoclonal antibody can, for example, be determined by Scatchard analysis (Munson, P. J. et al., Ligand: a versatile computerized approach for characterization of ligand-binding systems. Anal Biochem. 1980 Sep. 1; 107(1):220-39). In some cases, antibody specificity for regions of a given antigen may be characterized by chemically modifying the antigens prior to assaying for antibody binding. In one example, periodate treatment may be used to destroy the C6 side chain of sialic acids. Assays may be conducted with and without periodate treatment to reveal whether or not binding in untreated samples is sialic acid-specific. In some cases, antigens having 9-O-acetylated sialic acid may be subjected to mild base treatment (e.g. with 0.1 M NaOH) to destroy 9-O-acetyl groups. Assays may be conducted with and without mild base treatment to reveal whether or not binding in untreated samples depends on 9-O-acetylation of sialic acid.

After the desired hybridoma cells are identified, the clones may be subcloned by limiting dilution procedures and grown by standard methods. Suitable culture media for this purpose include, for example, Dulbecco's Modified Eagle's Medium or RPMI-1640 medium. Alternatively, the hybridoma cells may be grown in vivo as ascites in a mammal.

Alternative methods to clone hybridomas may include those provided by kits from STEMCELL™ Technologies (Vancouver, BC, Canada), e.g. CLONACELL™-HY kit, containing methylcellulose-based semi-solid medium and other media and reagents, to support the selection and growth of hybridoma clones. However, the media in this kit contain FCS, which provides an exogenous source for Neu5Gc incorporation. Though the machinery for endogenous Neu5Gc synthesis is destroyed in Cmah^{-/-} hybridoma, Neu5Gc incorporated from the culture media may also pose a problem in some cases (Bardor, M. et al., Mechanism of uptake and incorporation of the non-human sialic acid N-glycolylneuraminic acid into human cells. J Biol Chem. 2005. 280: 4228-4237). In such instances, the culture media may be supplemented with Neu5Ac to eliminate Neu5Gc incorporation by metabolic competition (Ghaderi, D. et al., Implications of the presence of N-glycolylneuraminic acid in recombinant therapeutic glycoproteins. Nat Biotechnol. 2010. 28: 863-867).

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In another embodiment, the monoclonal antibodies of the present invention can also be made by recombinant DNA methods, such as those described in U.S. Pat. No. 4,816,567, which is hereby incorporated by reference in its entirety. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of DNA. Once isolated, the DNA can be placed into expression vectors, which are then transfected into host cells. Host cells may include, but are not limited to HEK293 cells, HEK293T cells, simian COS cells, Chinese hamster ovary (CHO) cells, and myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiments, antibodies of the present invention (e.g., glycan-interacting antibodies) may be produced by various procedures known by those skilled in the art. For the production of polyclonal antibodies in vivo, host animals, such as rabbits, rats, mice, cows, horses, donkeys, chickens, monkeys, sheep or goats, are immunized with either free or carrier-coupled antigens, for example, by intraperitoneal and/or intradermal injection. In some embodiments, injection material may be an emulsion containing about 100 μg of antigen or carrier protein. In some embodiments, injection materials may include a glycan-rich composition such as non-human mammalian submaxillary mucin in solution. Various adjuvants can also be used to increase the immunological response, depending on the host species. Adjuvants include, but are not limited to, Freund's (complete and incomplete), mineral gels such as aluminum hydroxide, surface active substances such as lysolecithin, pluronic polyols, polyanions, peptides, oil emulsions, TITERMAX® (CytRx Corp, Los Angeles, CA), keyhole limpet hemocyanins, dinitrophenol, and potentially useful human adjuvants such as BCG (bacille Calmette-Guerin) and Corynebacterium parvum. Such adjuvants are also well known in the art. Several booster injections may be needed, for instance, at intervals of about two weeks, to provide a useful titer of antibody which can be detected, for example, by ELISA assay using glycans and/or free peptide adsorbed to a solid surface. The titer of antibodies in serum from an immunized animal can be increased by selection of antibodies, e.g., by adsorption of antigens onto a solid support and elution of the selected antibodies according to methods well known in the art.

Glycan-interacting antibodies, variants and fragments thereof may be selected and produced using high throughput methods of discovery. In one embodiment, glycan-interacting antibodies that include synthetic antibodies, variants and fragments thereof are produced through the use of display libraries. The term “display” as used herein, refers to the expression or “display” of proteins or peptides on the surface of a given host. The term “library” as used herein, refers to a collection of unique cDNA sequences and/or the proteins that are encoded by them. A library may contain from as little as two unique cDNAs to hundreds of billions of unique cDNAs. In some embodiments, glycan-interacting antibodies that are synthetic antibodies are produced using antibody display libraries or antibody fragment display libraries. The term “antibody fragment display library” as used herein, refers to a display library wherein each member encodes an antibody fragment containing at least one variable region of an antibody. Such antibody fragments are preferably Fab fragments, but other antibody fragments such as single-chain variable fragments (scFvs) are contemplated as well. In a Fab antibody fragment library, each Fab encoded may be identical except for the amino acid sequence contained within the variable loops of the complementarity determining regions (CDRs) of the Fab fragment. In an alternative or additional embodiment, amino acid sequences within the individual V_{H} and/or V_{L} regions may differ as well.

Display libraries may be expressed in a number of possible hosts including, but not limited to yeast, bacteriophage, bacteria and retroviruses. Additional display technologies that may be used include ribosome-display, microbead-display and protein-DNA linkage techniques. In a preferred embodiment, Fab display libraries are expressed in yeast or in bacteriophages (also referred to herein as “phages” or “phage particles”. When expressed, the Fabs decorate the surface of the phage or yeast where they can interact with a given antigen. An antigen that includes a glycan or other antigen from a desired target may be used to select phage particles or yeast cells expressing antibody fragments with the highest affinity for that antigen. The DNA sequence encoding the CDR of the bound antibody fragment can then be determined through sequencing using the bound particle or cell. In one embodiment, positive selection is used in the development of antibodies. In some embodiments, negative selection is utilized in the development of antibodies. In some embodiments, both positive and negative selection methods are utilized during multiple rounds of selection in the development of antibodies using display libraries.

In yeast display, cDNA encoding different antibody fragments are introduced into yeast cells where they are expressed and the antibody fragments are “displayed” on the cell surface as described by Chao et al. (Chao, G. et al., Isolating and engineering human antibodies using yeast surface display. Nat Protoc. 2006; 1(2):755-68). In yeast surface display, expressed antibody fragments may contain an additional domain that includes the yeast agglutinin protein, Aga2p. This domain allows the antibody fragment fusion protein to attach to the outer surface of the yeast cell through the formation of disulfide bonds with surface-expressed Aga1p. The result is a yeast cell, coated in a particular antibody fragment. Display libraries of cDNA encoding these antibody fragments are utilized initially in which the antibody fragments each have a unique sequence. These fusion proteins are expressed on the cell surface of millions of yeast cells where they can interact with a desired antigenic target antigen, incubated with the cells. Target antigens may be covalently or otherwise modified with a chemical or magnetic group to allow for efficient cell sorting after successful binding with a suitable antibody fragment takes place. Recovery may be by way of magnetic-activated cell sorting (MACS), fluorescence-activated cell sorting (FACS) or other cell sorting methods known in the art. Once a subpopulation of yeast cells is selected, the corresponding plasmids may be analyzed to determine the CDR sequence.

Bacteriophage display technology typically utilizes filamentous phage including, but not limited to fd, F1 and M13 virions. Such strains are non-lytic, allowing for continued propagation of the host and increased viral titers. Examples of phage display methods that can be used to make the antibodies of the present invention include those disclosed in Miersch et al. (Miersch, S. et al., Synthetic antibodies: Concepts, potential and practical considerations. Methods. 2012 August; 57(4):486-98), Bradbury et al. (Bradbury, A. R. et al., Beyond natural antibodies: the power of in vitro display technologies. Nat Biotechnol. 2011 March; 29(3):245-54), Brinkman et al. (Brinkmann, U. et al., Phage display of disulfide-stabilized Fv fragments. J Immunol Methods. 1995 May 11; 182(1):41-50); Ames et al. (Ames, R. S. et al., Conversion of murine Fabs isolated from a combinatorial phage display library to full length immunoglobulins. J Immunol Methods. 1995 Aug. 18; 184(2):177-86); Kettleborough et al. (Kettleborough, C. A. et al., Isolation of tumor cell-specific single-chain Fv from immunized mice using phage-antibody libraries and the re-construction of whole antibodies from these antibody fragments. Eur J Immunol. 1994 April; 24(4):952-8); Persic et al. (Persic, L. et al., An integrated vector system for the eukaryotic expression of antibodies or their fragments after selection from phage display libraries. Gene. 1997 Mar. 10; 187(1):9-18); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, each of which is incorporated herein by reference in its entirety. Antibody fragment expression on bacteriophages may be carried out by inserting the cDNA encoding the fragment into the gene expressing a viral coat protein. The viral coat of filamentous bacteriophages is made up of five coat proteins, encoded by a single-stranded genome. Coat protein pIII is the preferred protein for antibody fragment expression, typically at the N-terminus. If antibody fragment expression compromises the function of pIII, viral function may be restored through coexpression of a wild type pIII, although such expression will reduce the number of antibody fragments expressed on the viral coat, but may enhance access to the antibody fragment by the target antigen. Expression of viral as well as antibody fragment proteins may alternatively be encoded on multiple plasmids. This method may be used to reduce the overall size of infective plasmids and enhance the transformation efficiency.

As described above, after selection of a host expressing a high affinity antibody or antibody fragment, (e.g., glycan-interacting antibodies) the coding regions from the antibody or antibody fragment can be isolated and used to generate whole antibodies, including human antibodies, or any other desired antigen binding fragment, and expressed in any desired host, including mammalian cells, insect cells, plant cells, yeast, and bacteria, e.g., as described in detail below.

The DNA sequence encoding a high affinity antibody can be mutated for additional rounds of selection in a process known as affinity maturation. The term “affinity maturation”, as used herein, refers to a method whereby antibodies are produced with increasing affinity for a given antigen through successive rounds of mutation and selection of antibody- or antibody fragment-encoding cDNA sequences. In some cases, this process is carried out in vitro. To accomplish this, amplification of CDR coding sequences may be carried out using error-prone PCR to produce millions of copies containing mutations including, but not limited to point mutations, regional mutations, insertional mutations and deletional mutations. As used herein, the term “point mutation” refers to a nucleic acid mutation in which one nucleotide within a nucleotide sequence is changed to a different nucleotide. As used herein, the term “regional mutation” refers to a nucleic acid mutation in which two or more consecutive nucleotides are changed to different nucleotides. As used herein, the term “insertional mutation” refers to a nucleic acid mutation in which one or more nucleotides are inserted into a nucleotide sequence. As used herein, the term “deletional mutation” refers to a nucleic acid mutation in which one or more nucleotides are removed from a nucleotide sequence. Insertional or deletional mutations may include the complete replacement of an entire codon or the change of one codon to another by altering one or two nucleotides of the starting codon.

Mutagenesis may be carried out on CDR-encoding cDNA sequences to create millions of mutants with singular mutations in CDR heavy and light chain regions. In another approach, random mutations are introduced only at CDR residues most likely to improve affinity. These newly generated mutagenic libraries can be used to repeat the process to screen for clones that encode antibody fragments with even higher affinity for the target antigen. Continued rounds of mutation and selection promote the synthesis of clones with greater and greater affinity (Chao, G. et al., Isolating and engineering human antibodies using yeast surface display. Nat Protoc. 2006; 1(2):755-68).

Examples of techniques that can be used to produce antibodies and antibody fragments, such as Fabs and scFvs, include those described in U.S. Pat. Nos. 4,946,778 and 5,258,498; Miersch et al. (Miersch, S. et al., Synthetic antibodies: Concepts, potential and practical considerations. Methods. 2012 August; 57(4):486-98), Chao et al. (Chao, G. et al., Isolating and engineering human antibodies using yeast surface display. Nat Protoc. 2006; 1(2):755-68), Huston et al. (Huston, J. S. et al., Protein engineering of single-chain Fv analogs and fusion proteins. Methods Enzymol. 1991; 203:46-88); Shu et al. (Shu, L. et al., Secretion of a single-gene-encoded immunoglobulin from myeloma cells. Proc Natl Acad Sci USA. 1993 Sep. 1; 90(17):7995-9); and Skerra et al. (Skerra, A. et al., Assembly of a functional immunoglobulin Fv fragment in Escherichia coli. Science. 1988 May 20; 240(4855):1038-41), each of which is incorporated herein by reference in its entirety.

For some uses, including the in vivo use of antibodies (e.g., glycan-interacting antibodies) in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal immunoglobulin and a human immunoglobulin constant region. Methods for producing chimeric antibodies are known in the art. (Morrison, S. L., Transfectomas provide novel chimeric antibodies. Science. 1985 Sep. 20; 229(4719):1202-7; Gillies, S. D. et al., High-level expression of chimeric antibodies using adapted cDNA variable region cassettes. J Immunol Methods. 1989 Dec. 20; 125(1-2):191-202.; and U.S. Pat. Nos. 5,807,715; 4,816,567; and 4,816,397, which are incorporated herein by reference in their entirety).

Humanized antibodies are antibody molecules from non-human species that bind to the desired antigen and have one or more complementarity determining regions (CDRs) from the nonhuman species and framework regions from a human immunoglobulin molecule. Often, framework residues in the human framework regions are substituted with corresponding residues from the CDR and framework regions of the donor antibody to alter, preferably improve, antigen binding. These framework substitutions are identified by methods well known in the art, e.g., by modeling of the interactions of the CDR and framework residues to identify framework residues important for antigen binding, and by sequence comparison to identify unusual framework residues at particular positions. (U.S. Pat. Nos. 5,693,762 and 5,585,089; Riechmann, L. et al., Reshaping human antibodies for therapy. Nature. 1988 Mar. 24; 332(6162):323-7, which are incorporated herein by reference in their entireties). Antibodies can be humanized using a variety of techniques known in the art, including, for example, CDR-grafting (EP 239,400; PCT publication WO 91/09967; U.S. Pat. Nos. 5,225,539; 5,530,101; and 5,585,089); veneering or resurfacing (EP 592,106; EP 519,596; Padlan, E. A., A possible procedure for reducing the immunogenicity of antibody variable domains while preserving their ligand-binding properties. Mol Immunol. 1991 April-May; 28(4-5):489-98; Studnicka, G. M. et al., Human-engineered monoclonal antibodies retain full specific binding activity by preserving non-CDR complementarity-modulating residues. Protein Eng. 1994 June; 7(6):805-14; Roguska, M. A. et al., Humanization of murine monoclonal antibodies through variable domain resurfacing. Proc Natl Acad Sci USA. 1994 Feb. 1; 91(3):969-73); and chain shuffling (U.S. Pat. No. 5,565,332); each of which is incorporated herein by reference in their entirety. Humanized antibodies of the present invention may be developed for desired binding specificity, complement-dependent cytotoxicity, and antibody-dependent cellular-mediated cytotoxicity, etc.

In some cases, human frameworks are selected by alignment of donor antibody sequences with human framework sequences to find human framework candidates with the highest level of homology. In some cases, framework regions may be selected from more than one human framework candidate (e.g., framework regions 1-3 may be selected from one candidate and framework region 4 may be selected from an alternative candidate). In some cases, framework regions may be selected from human consensus sequences to avoid the risk of including immunogenic epitopes created by somatic mutations. Consensus sequences are sequences formed by comparing many sequences and adopting most commonly occurring residues at each position. In some cases, human frameworks may be selected from human germline sequences. These may be identified through database searching (e.g., using the NCBI protein database or other databases).

Light and heavy chain human frameworks may be selected from the same or from different clones. Light and heavy chains derived from the same clone have a greater likelihood of associating to form binding sites that are functional; however, the conserved nature of the interface between heavy and light chains typically allows light and heavy chains from different clones to associate and be functional. Frequency of pairing between human light and heavy chain frameworks can be reviewed, for example, in Tiller et al., 2013. MAbs. 5(3): 445-70, the contents of which are herein incorporated by reference in their entirety.

Residues in humanized antibody sequences may be considered for “back-mutation” to improve or restore antibody affinity lost during humanization. Back-mutation involves changing residues altered during humanization back to those present in the original non-human antibody sequence. Residues that are candidates for back-mutation may be identified, for example, by comparison to standard conformations found in canonical antibody structures (see Al-Lazikani, et al., 1997. J. Mol. Biol. 273: 927-48, the contents of which are herein incorporated by reference in their entirety). Unusual canonical residues may be identified and targeted for back-mutation. In some cases, residues that are candidates for back-mutation may be “Vernier residues”, a term used to refer to residues in contact with CDRs. These residues have a higher likelihood of impacting CDR positioning and conformation, and therefor antibody affinity and/or specificity (Strohl, W. R. Therapeutic Antibody Engineering. Woodhead Publishing, Philadelphia PA. 2012. Ch. 6, p 117). In some cases, human framework regions are kept constant and CDRs from donor antibodies are back-mutated to fit human CDR regions while maintaining binding through empirical methods.

Completely human antibodies (e.g., glycan-interacting antibodies) are particularly desirable for therapeutic treatment of human patients, so as to avoid or alleviate immune reaction to foreign protein. Human antibodies can be made by a variety of methods known in the art, including the antibody display methods described above, using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies (e.g., glycan-interacting antibodies) can also be produced using transgenic mice which are incapable of expressing functional endogenous immunoglobulins, but which can express human immunoglobulin polynucleotides. For example, the human heavy and light chain immunoglobulin polynucleotide complexes can be introduced randomly, or by homologous recombination, into mouse embryonic stem cells. Alternatively, the human variable region, constant region, and diversity region may be introduced into mouse embryonic stem cells, in addition to the human heavy and light chain polynucleotides. The mouse heavy and light chain immunoglobulin polynucleotides can be rendered nonfunctional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a glycan, glycoconjugate and/or polypeptide of the invention.

Thus, using such a technique, it is possible to produce useful human IgG, IgA, IgM, IgD and IgE antibodies. For an overview of the technology for producing human antibodies, see Lonberg and Huszar (Lonberg, N. et al., Human antibodies from transgenic mice. Int Rev Immunol. 1995; 13(1):65-93). For a detailed discussion of the technology for producing human antibodies and human monoclonal antibodies and protocols for producing such antibodies, see, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; 5,939,598; 6,075,181; and 6,114,598, each of which are incorporated by reference herein in their entirety. In addition, companies such as Abgenix, Inc. (Fremont, Calif), Protein Design Labs, Inc. (Mountain View, Calif.) and Genpharm (San Jose, Calif.) can be engaged to provide human antibodies directed against a selected antigen using technology similar to the above described technologies.

Once an antibody molecule of the present invention has been produced by an animal, a cell line, chemically synthesized, or recombinantly-expressed, it can be purified (i.e., isolated) by any method known in the art for the purification of an immunoglobulin or polypeptide molecule, for example, by chromatography (e.g., ion exchange, affinity, particularly by affinity for the specific antigen, Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The affinity between an antibody and a target or ligand (such as an antigen used to generate a given antibody) may be measured in terms of K_{D} using one or more binding assays as described herein. Depending on the desired application for a given antibody, varying K_{D} values may be desirable. High affinity antibodies typically form ligand bonds with a K_{D} of about 10⁻⁵ M or less, e.g. about 10⁻⁶ M or less, about 10⁻⁷ M or less, about 10⁻⁸ M or less, about 10⁻⁹ M or less, about 10⁻¹⁰ M or less, about 10⁻¹¹ M or less or about 10⁻¹² M or less.

In some embodiments, antibodies of the invention may be characterized according to their half maximal effective or inhibitory concentration (EC₅₀ or IC₅₀, respectively). In some cases, this value may represent the concentration of antibody necessary to inhibit cells expressing STn (e.g. kill, reduce proliferation and/or reduce one or more cell function) at a level equal to half of the maximum inhibition observed with the highest concentrations of antibody. Such IC₅₀ values may be from about 0.001 nM to about 0.01 nM, from about 0.005 nM to about 0.05 nM, from about 0.01 nM to about 1 nM, from about 0.05 nM to about 5 nM, from about 0.1 nM to about 10 nM, from about 0.5 nM to about 25 nM, from about 1 nM to about 50 nM, from about 5 nM to about 75 nM, from about 10 nM to about 100 nM, from about 25 nM to about 250 nM, from about 200 nM to about 1000 nM or more than 1000 nM.

In some embodiments, antibodies taught in the present disclosure may be tested for their ability to target patient-derived cancer cells and/or cancer stem cells (CSCs). According to such embodiments, patient-derived cancer cells may be cultured in vitro and antibodies of the present disclosure may be used to target such cells.

In other embodiments, patient-derived cells may be used to produce patient-derived xenograft (PDX) tumors. In some cases, pieces of primary or metastatic solid tumors maintained as tissue structures may be collected by surgery or biopsy procedures. In some cases, fluid drained from malignant ascites or pleural effusions may be used. Tumors may be implanted as pieces or single cell suspensions, either alone or in some studies coated with MATRIGEL® (Corning Life Sciences, Corning, NY) or mixed with human fibroblasts or mesenchymal stem cells. Sites of implantation may include the dorsal region of mice (subcutaneous implantation), although implantation in the same organ as the original tumor may be an option (orthotopic implantation, i.e. pancreas, oral cavity, ovary, mammary fat pad, brain, etc.). In addition, independently of the tumor origin, some approaches may include implanting primary tumors in the renal capsule in an effort to increase engraftment success rates. A variety of mouse strains having different degrees of immunosuppression may be used in such studies. For hormone sensitive tumors, some studies may use hormone supplementation with the intent of increasing engraftment rates. In some embodiments, PDX tumors may be generated in non-obese diabetic/severe combined immunodeficiency (NOD/SCID) mice. Antibodies may be administered to mice with PDX tumors and the effect on tumor volume may be analyzed. In some cases, PDX tumors may be dissected, subjected to cellular dissociation, and the resulting cells grown in culture. The ability of antibodies of the present disclosure to target these cells may be assessed in vitro.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

### Targets

Glycan-interacting antibodies of the present invention may exert their effects via binding (reversibly or irreversibly) to one or more glycan or glycan-associated or glycan-related targets. In some embodiments, glycan-interacting antibodies can be prepared from any region of the targets taught herein. In some embodiments, targets of the present invention include glycans. Glycans used for generating antibodies may include a chain of sugars having at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 residues. Some glycans used for generating antibodies may include from about 2 residues to about 5 residues.

In some embodiments, glycan-interacting antibody target antigens include sialic acids. N-acetylneuraminic acid (Neu5Ac) and N-glycolylneuraminic acid (Neu5Gc) are the major sialic acids on mammalian cell surfaces. Of these, Neu5Ac is naturally produced in humans. Neu5Gc is naturally produced in most mammals with the exception of humans due to a mutation in the cytidine monophosphate (CMP)-N-acetylneuraminic acid hydroxylase (CMAH) gene responsible for CMP-Neu5Gc production from CMP-Neu5Ac. Neu5Gc in humans is in fact immunogenic with nearly all humans expressing anti-Neu5Gc antibodies. Despite a lack of production, most human systems include some level of Neu5Gc due to dietary intake. These foreign products are subsequently incorporated into human glycoproteins. Such glycoproteins are contemplated as targets of the invention. Glycan target antigens of the present invention may include, but are not limited to those listed in Table 1.

TABLE 1
Glycan target antigens
Glycan

GalNAcα-R
Galα1,3Galβ1,4GlcNAcβ-R
Galβ1,3GalNAcβ-R
Galβ1,3GlcNAcα-R
Galβ1,3GlcNAcβ1,3Galβ1,4Glcβ-R
Galβ1,3GlcNAcβ-R
Galβ1,4GlcNAc6Sβ-R
Galβ1,4GlcNAcβ-R
Galβ1,4Glcβ-R
KDNα2,8Neu5Acα2,3Galβ1,4Glcβ-R
KDNα2,8Neu5Gcα2,3Galβ1,4Glcβ-R
Neu5,9Ac2α2,3Galβ1,3GalNAcα-R
Neu5,9Ac2α2,3Galβ1,3GalNAcβ-R
Neu5,9Ac2α2,3Galβ1,3GlcNAcβ-R
Neu5,9Ac2α2,3Galβ1,4GlcNAcβ-R
Neu5,9Ac2α2,3Galβ1,4Glcβ-R
Neu5,9Ac2α2,3Galβ-R
Neu5,9Ac2α2,6GalNAcα-R
Neu5,9Ac2α2,6Galβ1,4GlcNAcβ-R
Neu5,9Ac2α2,6Galβ1,4Glcβ-R
Neu5,9Ac2α2,6Galβ-R
Neu5Acα2,3Galβ1,3GalNAcα-R
Neu5Acα2,3Galβ1,3GalNAcβ-R
Neu5Acα2,3Galβ1,3GlcNAcβ1,3Galβ1,4Glcβ-R
Neu5Acα2,3Galβ1,3GlcNAcβ-R
Neu5Acα2,3Galβ1,4(Fucα1,3)GlcNAc6Sβ-R
Neu5Acα2,3Galβ1,4(Fucα1,3)GlcNAcβ-R
Neu5Acα2,3Galβ1,4GlcNAc6Sβ-R
Neu5Acα2,3Galβ1,4GlcNAcβ-R
Neu5Acα2,3Galβ1,4Glcβ-R
Neu5Acα2,3Galβ-R
Neu5Acα2,6(KDNα2,3)Galβ1,4Glcβ-R
Neu5Acα2,6(Neu5Acα2,3)Galβ1,4Glcβ-R
Neu5Acα2,6(Neu5Gcα2,3)Galβ1,4Glcβ-R
Neu5Acα2,6GalNAcα-R
Neu5Acα2,6Galβ1,4GlcNAcβ-R
Neu5Acα2,6Galβ1,4Glcβ-R
Neu5Acα2,6Galβ-R
Neu5Acα2,8KDNα2,6Galβ1,4Glcβ-R
Neu5Acα2,8Neu5Acα2,3Galβ1,4Glcβ-R
Neu5Acα2,8Neu5Acα2,3Galβ1,4Glcβ-R
Neu5Acα2,8Neu5Acα2,6Galβ1,4Glcβ-R
Neu5Acα2,8Neu5Acα2,8Neu5Acα2,3Galβ1,4Glcβ-R
Neu5Acα2,8Neu5Acα2,8Neu5Acα2,3Galβ1,4Glcβ-R
Neu5Acα2,8Neu5Gcα2,3Galβ1,4Glcβ-R
Neu5Acα2,8Neu5Gcα2,6Galβ1,4Glcβ-R
Neu5Gc9Acα2,3Galβ1,4Glcβ-R
Neu5Gc9Acα2,6Galβ1,4Glcβ-R
Neu5Gc9Acα2,3Galβ1,3GalNAcα-R
Neu5Gc9Acα2,3Galβ1,3GalNAcβ-R
Neu5Gc9Acα2,3Galβ1,3GlcNAcβ-R
Neu5Gc9Acα2,3Galβ1,4GlcNAcβ-R
Neu5Gc9Acα2,3Galβ-R
Neu5Gc9Acα2,6GalNAcα-R
Neu5Gc9Acα2,6Galβ1,4GlcNAcβ-R
Neu5Gc9Acα2,6Galβ-R
Neu5GcOMeα2,8Neu5Acα2,3Galβ1,4Glcβ-R
Neu5Gcα2,3Galβ1,3GalNAcα-R
Neu5Gcα2,3Galβ1,3GalNAcβ-R
Neu5Gcα2,3Galβ1,3GlcNAcβ1,3Galβ1,4Glcβ-R
Neu5Gcα2,3Galβ1,3GlcNAcβ-R
Neu5Gcα2,3Galβ1,4(Fucα1,3)GlcNAc6Sβ-R
Neu5Gcα2,3Galβ1,4(Fucα1,3)GlcNAcβ-R
Neu5Gcα2,3Galβ1,4GlcNAc6Sβ-R
Neu5Gcα2,3Galβ1,4GlcNAcβ-R
Neu5Gcα2,3Galβ1,4Glcβ-R
Neu5Gcα2,3Galβ-R
Neu5Gcα2,6GalNAcα-R
Neu5Gcα2,6Galβ1,4GlcNAcβ-R
Neu5Gcα2,6Galβ1,4Glcβ-R
Neu5Gcα2,6Galβ-R
Neu5Gcα2,8Neu5Acα2,3Galβ1,4Glcβ-R
Neu5Gcα2,8Neu5Gcα2,3Galβ1,4Glcβ-R

The following abbreviations are used herein: Glc—glucose, Gal—galactose, GlcNAcβ—N-acetylglucosamine, GalNAcβ—N-acetylgalactosamine, GlcNAc6S—6-Sulfo-N-acetylglucosamine, KDN—2-keto-3-deoxy-D-glycero-D-galactonononic acid, Neu5,9Ac2—N-acetyl-9-O-acetylneuraminic acid, Fuc—fucose and Neu5GcOMe—2-O-methyl-N-glycolylneuraminic acid. O-glycosidic bonds are present between each residue in the glycans listed with α and β indicating the relative stoichiometry between the two residues joined by the bond, wherein α indicates an axial orientation and β indicates an equatorial orientation. The numbers following α and/or β, in the format x,x, indicate the carbon number of each of the carbons from each of the adjoined residues that participate in bond formation. While the glycans listed in Table 1 represent individual glycan target antigens contemplated, the present invention also includes embodiments wherein the above presented glycans include different combinations of α and β-oriented O-glycosidic bonds than the ones presented. Also in Table 1, R represents an entity that the glycan may be coupled with. In some embodiments, R is a protein wherein the glycan is linked typically to a serine or threonine residue. In some embodiments, R is a linker molecule used to join the glycan to a substrate, such as in a glycan array. In some embodiments, R may be a linker with the formula of —(CH₂)₂CH₂NH₂ or —(CH₂)₃NHCOCH₂(OCH₂CH₂)₆NH₂. In some embodiments, R may be biotin, albumin, ProNH₂, —CH—, —OH, —OCH₃, —OCH₂CH₃, —H, hydrido, hydroxy, alkoxyl, oxygen, carbon, sulfur, nitrogen, polyacrylamide, phosphorus, NH₂, ProNH₂═O(CH₂)₂CH₂NH₂, (OCH₂CH₂)₆NH₂, O(CH₂)₃NHCOCH₂(OCH₂CH₂)₆NH₂, the fluorescent labels 2-aminobenzamide (AB) and/or 2-aminobenzoid acid (AA), 2-aminobenzamide analog that contains an alkyl amine (AEAB), aminooxy-groups, methylaminooxygroups, hydrazide groups, amino lipid 1,2-dihexadecyl-sn-glycero-3-phosphoethanolamine (DHPE), aminooxy (AO) functionalized DHPE and glycosylphosphatidylinositol (GPI). Without intending to limit the source or nature of R, this may include structures that affect the physical spacing of glycan residue. In some embodiments, the R group may include a combination of the R groups presented here, e.g. a biotinylated polyacrylamide. In some embodiments, the R group in combination with underlying substrates effect glycan residue spacing.

Glycan targets of the present invention may include one or more regions of antibody recognition. As used herein, the term “region of antibody recognition” refers to a segment located on any part of the molecule, an attached group or located on a region of interaction between the glycan and another molecule, including, but not limited to another glycan, protein, membrane, cell surface structure, or extracellular matrix component. In some embodiments, regions of antibody recognition are located at interchain target sites, wherein the term “interchain” means within the present polymeric chain. Interchain target sites may include regions of antibody recognition having 1, 2, 3, 4, 5, 6, 7, 8, 9 or at least 10 residues, bonds between residues or combinations of residues and bonds. In some embodiments, regions of antibody recognition are located at regions of interaction between one or more glycan chains. Such regions may be between 2, 3, 4 or at least 5 glycan chains.

In some embodiments, regions of antibody recognition are located at regions of interaction between glycan branch chains connected to a common parent chain. In some embodiments, regions of antibody recognition are located at regions of interaction between a glycan branch chain and a parent chain. In some embodiments, regions of antibody recognition are located at regions of interaction between glycans and proteins. Such regions of interaction may include chemical bonds between the glycan and the protein, including, but not limited to covalent bonds, ionic bonds, hydrostatic bonds, hydrophobic bonds and hydrogen bonds. In some embodiments, regions of antibody recognition are located at regions of interaction between glycans and other biomolecules including, but not limited to lipids and nucleic acids. Such regions of interaction may include chemical bonds between the glycan and the biomolecule, including, but not limited to covalent bonds, ionic bonds, hydrostatic bonds, hydrophobic bonds and hydrogen bonds.

In some embodiments, glycan targets of the present invention are components of glycoconjugates. As used herein, the term “glycoconjugate” refers to any entity joined with a glycan moiety. In some embodiments, glycoconjugates are glycolipids. As used herein, the term “glycolipid” refers to a class of lipids wherein a carbohydrate moiety is covalently attached. In some embodiments, carbohydrate moieties present on glycolipids may be glycans. In some embodiments, lipid components of glycolipids include ceramide moieties. Examples of glycolipids contemplated as targets of the present invention include, but are not limited to glyceroglycolipids (including, but not limited to galactolipids and sulfolipids), glycosphingolipids (including, but not limited to cerebrosides (e.g., galactocerebrosides, glucocerebrosides and sulfatides), gangliosides, globosides and glycophosphosphingolipids) and glycosylphosphatidylinositols. When located within cell membranes, glycan moieties of glycolipids are located on the extracellular side of the membrane where they may interact with other cells as well as cell signaling ligands (Maccioni, H. J. et al., Organization of the synthesis ofglycolipid oligosaccharides in the Golgi complex. FEBS Lett. 2011 Jun. 6; 585(11):1691-8).

In some embodiments, glycoconjugate targets of the present invention are glycoprotein and/or proteoglycans. Glycoproteins refer to any proteins that are covalently bonded with glycans. Proteoglycans are a class of proteins that are heavily glycosylated with glycans that often carry a negative charge. This property makes them very hydrophilic and important components of connective tissue.

### Cancer-Related Targets

In some embodiments, targets of the present invention are cancer-related antigens or epitopes. As used herein, the term “cancer-related” is used to describe entities that may be in some way associated with cancer, cancerous cells and/or cancerous tissues. Many cancer-related antigens or epitopes that include glycans have been identified that are expressed in correlation with tumor cells (Heimburg-Molinaro, J. et al., Cancer vaccines and carbohydrate epitopes. Vaccine. 2011 Nov. 8; 29(48):8802-26). These are referred to herein as “tumor-associated carbohydrate antigens” or “TACAs.” TACAs include, but are not limited to mucin-related antigens [including, but not limited to Tn, Sialyl Tn (STn) and Thomsen-Friedenreich antigen], blood group Lewis related antigens [including, but not limited to Lewis^{Y} (Le^{Y}), Lewis^{X} (Le^{X}), Sialyl Lewis^{X} (SLe^{X}) and Sialyl Lewis^{A} (SLe^{A})], glycosphingolipid-related antigens [including, but not limited to Globo H, stage-specific embryonic antigen-3 (SSEA-3) and glycosphingolipids that include sialic acid], ganglioside-related antigens [including, but not limited to gangliosides GD2, GD3, GM2, fucosyl GM1 and Neu5GcGM3] and polysialic acid-related antigens. Many of such antigens are described in International Publication No. WO2015054600, the contents of which are herein incorporated by reference in their entirety.

In some embodiments, TACA targets of the present invention include Lewis blood group antigens. Lewis blood group antigens include a fucose residue linked to GlcNAc by an α1-3 linkage or an α1-4 linkage. They may be found on both glycolipids and glycoproteins. Lewis blood group antigens may be found in the body fluid of individuals that are secretors of these antigens. Their appearance on red cells is due to absorption of Lewis antigens from the serum by the red cells.

In some embodiments, TACA targets of the present invention include Le^{Y}. Le^{Y} (also known as CD174) is made up of Galβ1,4GlcNAC having α1,2- as well as α1,3-linked fucose residues yielding the Fucα(1,2)Galβ(1,4)Fucα(1,3)GlcNAc epitope. It is synthesized from the H antigen by α1,3 fucosyltransferases which attach the α1,3 fucose to the GlcNAc residue of the parent chain. Le^{Y} may be expressed in a variety of cancers including, but not limited to ovarian, breast, prostate, colon, lung and epithelial. Due to its low expression level in normal tissues and elevated expression level in many cancers, the Le^{Y} antigen is an attractive target for therapeutic antibodies.

In some embodiments, TACA targets of the present invention include Le^{X}. Le^{X} includes the epitope Galβ1-4(Fucα1-3)GlcNAcβ-R. It is also known as CD15 and stage-specific embryonic antigen-1 (SSEA-1). This antigen was first recognized as being immunoreactive with sera taken from a mouse subjected to immunization with F9 teratocarcinoma cells. Le^{X} was also found to correlate with embryonic development at specific stages. It is also expressed in a variety of tissues both in the presence and absence of cancer, but can also be found in breast and ovarian cancers where it is only expressed by cancerous cells.

In some embodiments, TACA targets of the present invention include SLe^{A} and/or SLe^{X}. SLe^{A} and SLe^{X} are made up of structures Neu5Acα2-3Galβ1-3(Fucα1-4)GlcNAcβ-R and Neu5Acα2-3Galβ1-4(Fucα1-3)GlcNAcβ-R, respectively. Their expression is upregulated in cancer cells. The presence of these antigens in serum correlates with malignancy and poor prognosis. SLe^{X} is mostly found as a mucin terminal epitope. It is expressed in a number of different cancers including breast, ovarian, melanoma, colon, liver, lung and prostate. In some embodiments of the present invention, SLe^{A} and SLe^{X} targets include Neu5Gc (referred to herein as GcSLe^{A} and GcSLe^{X}, respectively).

In some cases, cancer-related targets of the invention may include mucins. Ishida et al demonstrate that interaction of MUC2 with dendritic cells (with anti-tumor activity) leads to dendritic cell apoptosis (Ishida, A. et al., 2008. Proteomics. 8: 3342-9, the contents of which are herein incorporated by reference in their entirety). In some aspects, the present invention provided anti-mucin antibodies to prevent dendritic cell apoptosis and support anti-tumor activity.

In some embodiments, TACA targets of the present invention include glycolipids and/or epitopes present on glycolipids, including, but not limited to glycosphingolipids. Glycosphingolipids include the lipid ceramide linked to a glycan by the ceramide hydroxyl group. On the cell membrane, glycosphingolipids form clusters referred to as “lipid rafts”.

In some embodiments, TACA targets of the present invention include Globo H. Globo H is a cancer-related glycosphingolipid first identified in breast cancer cells. The glycan portion of Globo H includes Fucα(1-2)Galβ(1-3)GalNAcβ(1-3)Galα(1-4)Galβ(1-4)Glcβ(1). Although found in a number of normal epithelial tissues, Globo H has been identified in association with many tumor tissues including, but not limited to, small cell lung, breast, prostate, lung, pancreatic, gastric, ovarian and endometrial tumors.

In some embodiments, cancer-related glycosphingolipid targets of the present invention include gangliosides. Gangliosides are glycosphingolipids having one or more sialic acid. According to ganglioside nomenclature, G is used as an abbreviation for ganglioside. This abbreviation is followed by the letters M, D or T referring to the number of sialic acid residues attached (1, 2 or 3 respectively). Finally, the numbers 1, 2 or 3 are used to refer to the order of the distance each migrates when analyzed by thin layer chromatography (wherein 3 travels the greatest distance, followed by 2, and then 1). Gangliosides are known to be involved in cancer-related growth and metastasis and may be expressed on the cell surface of tumor cells. Gangliosides expressed on tumor cells may include, but are not limited to GD2, GD3, GM2 and fucosyl GM1 (also referred to herein as Fuc-GM1). In some embodiments of the present invention, glycan-interacting antibodies are directed toward GD3. GD3 is a regulator of cell growth. In some embodiments, GD3-directed antibodies are used to modulate cell growth and/or angiogenesis. In some embodiments, GD3-directed antibodies are used to modulate cell attachment. GD3 associated with some tumor cells may include 9-O-acetylated sialic acid residues (Mukherjee, K. et al., 2008. J Cell Biochem. 105: 724-34 and Mukherjee, K. et al., 2009. Biol Chem. 390: 325-35, the contents of each of which are herein incorporated by reference in their entirety). In some cases, antibodies of the invention are selective for 9-O-acetylated sialic acid residues. Some antibodies may be specific for 9-O-acetylated GD3s. Such antibodies may be used to target tumor cells expressing 9-O-acetylated GD3. In some embodiments of the present invention, glycan interacting antibodies are directed toward GM2. In some embodiments, GM2-directed antibodies are used to modulate cell to cell contact. In some embodiments, ganglioside targets of the present invention include Neu5Gc. In some embodiments, such targets may include a GM3 variant having Neu5Gc (referred to herein as GcGM3). The glycan component of GcGM3 is Neu5Gcα2-3Galβ1-4Glc. GcGM3 is a known component of tumor cells (Casadesus, A. V. et al., 2013. Glycoconj J. 30(7):687-99, the contents of which are herein incorporated by reference in their entirety).

In some embodiments, TACAs of the present disclosure include at least one Neu5Gc residue.

### Recombinant Antibodies

Recombinant antibodies (e.g., glycan-interacting antibodies) of the invention may be generated using standard techniques known in the art. In some embodiments, recombinant antibodies may be anti-glycan antibodies. Further antibodies may be anti-STn antibodies (e.g. anti-GcSTn or anti-AcSTn antibodies). Recombinant antibodies of the invention may be produced using variable domains obtained from hybridoma cell-derived antibodies produced according to methods described herein. Heavy and light chain variable region cDNA sequences of antibodies may be determined using standard biochemical techniques. Total RNA may be extracted from antibody-producing hybridoma cells and converted to cDNA by reverse transcriptase (RT) polymerase chain reaction (PCR). PCR amplification may be carried out on resulting cDNA to amplify variable region genes. Such amplification may include the use of primers specific for amplification of heavy and light chain sequences. In other embodiments, recombinant antibodies may be produced using variable domains obtained from other sources. This includes the use of variable domains selected from one or more antibody fragment library, such as an scFv library used in antigen panning. Resulting PCR products may then be subcloned into plasmids for sequence analysis. Once sequenced, antibody coding sequences may be placed into expression vectors. For humanization, coding sequences for human heavy and light chain constant domains may be used to substitute for homologous murine sequences. The resulting constructs may then be transfected into mammalian cells for large scale translation.

### Anti-Tn Antibodies

In some embodiments, recombinant antibodies of the invention (e.g., glycan-interacting antibodies) may be anti-Tn antibodies. Such antibodies may bind to targets having Tn. Anti-Tn antibodies may be specific for Tn or may bind other modified forms of Tn, such as Tn linked to other moieties, including, but not limited to additional carbohydrate residues. In some cases, anti-Tn antibodies may be anti-sialyl-Tn antibodies. Such antibodies may bind to sialylated Tn that includes Neu5Ac and/or sialylated Tn that include Neu5Gc. Some anti-Tn antibodies may bind specifically to clusters of Tn antigen.

### Anti-STn Antibodies

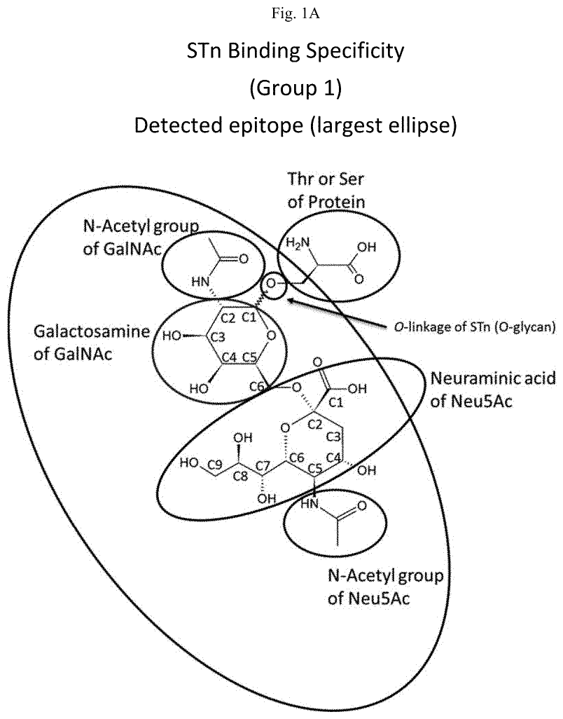

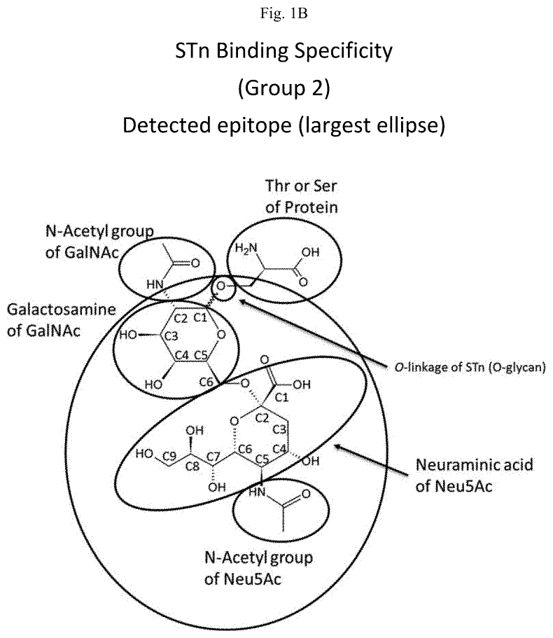

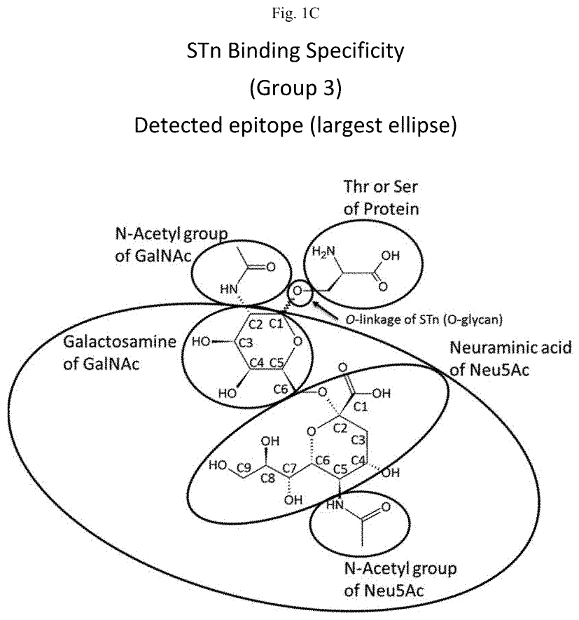

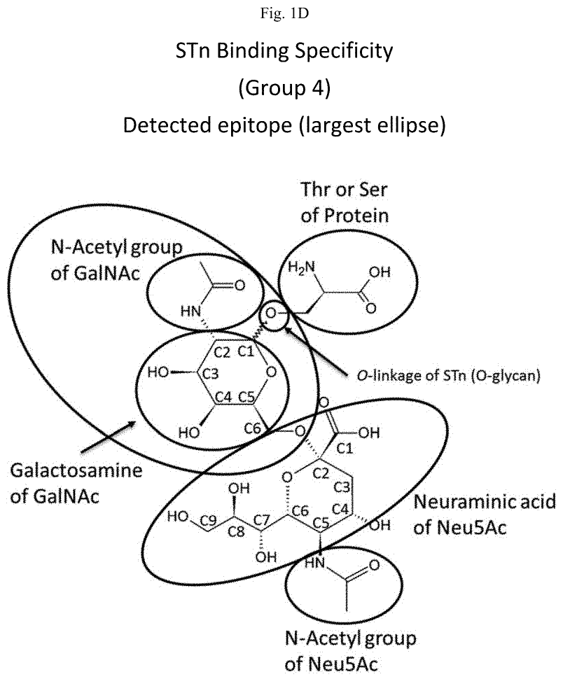

In some embodiments, antibodies of the invention (e.g., glycan-interacting antibodies) may specifically bind to STn. Anti-STn antibodies of the invention may be categorized by their binding to specific portions of STn antigens and/or by their specificity for AcSTn versus GcSTn. In some cases, anti-STn antibodies of the invention are Group 1 antibodies. “Group 1” antibodies according to the invention are antibodies capable of binding AcSTn and GcSTn. Such antibodies may also be referred to herein as pan-STn antibodies due to their ability to associate with a wider range of STn structures. In some embodiments, Group 1 antibodies may associate with the portion of STn indicated by the largest ellipse in FIG. 1A. In some cases, anti-STn antibodies of the invention are Group 2 antibodies. “Group 2” antibodies, according to the invention, are antibodies capable of binding STn as well as some related structures that include an O-linkage to serine or threonine. In some embodiments, Group 2 antibodies may associate with glycans that include a sialylated galactose residue. In some cases, Group 2 antibodies may associate with the portion of STn indicated by the largest ellipse in FIG. 1B. Some Group 2 antibodies preferably bind to structures with AcSTn over structures with GcSTn. Further anti-STn antibodies may be Group 3 antibodies. As referred to herein, “Group 3” antibodies are antibodies capable of binding STn, but may also bind a broader set of related structures. Unlike Group 2 antibodies, Group 3 antibodies do not require that such structures have an O-linkage to serine or threonine. In some embodiments, Group 3 antibodies may associate with the portion of STn indicated by the largest ellipse in FIG. 1C. Finally, some anti-STn antibodies of the invention may be Group 4 antibodies. As referred to herein, “Group 4” antibodies are capable of binding to both AcSTn and GcSTn as well as the un-sialylated Tn antigen, and therefore have broader specificity. In some embodiments, Group 4 antibodies may associate with the portion of STn indicated by the largest ellipse in FIG. 1D.

In some cases, anti-STn antibodies of the invention may bind specifically to clusters of STn on a particular antigen or cell surface. Some such antibodies may recognize epitopes formed by the clustering of STn, including epitopes that include areas of contact between neighboring STn structures. Such epitopes may be formed by the clustering of 2, 3, 4, 5, 6, 7, 8, 9, 10 or more STn structures.

In some embodiments, anti-STn antibodies of the present disclosure may be used bind cellular proteins carrying STn. Such antibodies may be useful for targeting cellular proteins associated with cancer cells that are distinguishable from similar proteins in non-cancerous cells by STn expression. In some cases, such proteins may include cell surface proteins. Cancer cell surface proteins carrying STn may be targeted by anti-STn antibodies during cancer treatment and/or diagnosis. Cell surface proteins carrying STn may be identified using mass spectrometry and/or using immunological methods (e.g., FACS analysis, immunoprecipitation, immunoblotting, ELISA, etc.). In some cases, cellular proteins carrying STn may include cancer cell markers, cancer stem cell markers, and/or cancer stem cell signaling proteins. In some embodiments, cellular proteins carrying STn may include, but are not limited to CD44, CD133, CD117, integrins, Notch, and Hedgehog.

### Antibody Components

Antibodies of the present disclosure may include any of the amino acid or nucleotide sequence presented herein, including, but not limited to variable domain sequences, CDR sequences, framework sequences, linker sequences, and immunoglobulin sequences. In some cases, antibodies may include any of the antibody or antibody fragment sequences presented in International Publication Number WO2017083582 (the entire content of which is herein incorporated by reference), including: any of the variable domain sequences presented in Table 2 therein; any of the CDR sequences presented in Table 3 therein; any of the VH CDR sequence groups presented in Table 4 therein; any of the VL CDR sequence groups presented in Table 5 therein; any of the variable domain nucleotide sequences presented in Table 6 therein; or any of the humanized variable domain sequences presented in Table 11 therein. Some antibodies or antigen binding fragments may include different combinations of such sequences.

In some embodiments, antibodies or antigen binding fragments of the invention may include one or more of the variable domain sequences listed in Table 2. Residues indicated with an “X” may be absent or selected from any amino acid residues. Light chain variable domains presented in the Table may be expressed with or without a C-terminal arginine residue. This residue typically links light chain variable domains with light chain constant domains and may be expressed as part of the light chain constant domain instead of the light chain variable domain. In some cases, antibodies or antigen binding fragments thereof may include an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the variable domain sequences listed in Table 2. In some cases, antibodies or antigen binding fragments thereof of the invention may include an amino acid sequence having one or more fragments of any of the sequences listed in Table 2.

TABLE 2
Variable domain sequences

| | | | SEQ |
|---|---|---|---|
| Antibody | Variable | | ID |
| ID | domain | Sequence | NO |
| SIA01 | Heavy | EVQLVQSGAEVKKPGASVKVSCKASGYTFTDHAIHWV | 1 |
| Number | chain | RQAPGQGLEWMGYFSPGNDDIKYNEKFRGRVTMTAD | |
| | | KSSSTAYMELRSLRSDDTAVYFCKRSLSTPYWGQGTLV | |
| | | TVSS | |
| SIA01 | Light | DIVMTQSPDSLAVSLGERATINCKSSQSLLNRGNHKNY | 2 |
| | chain | LTWYQQKPGQPPKLLIYWASTRESGVPDRFSGSGSGTD | |
| | | FTLTISSLQAEDVAVYYCONDYTYPYTFGQGTKVEIK | |

In some cases, antibodies or antigen binding fragments of the invention may include any of the IgG framework regions presented in Table 3. In some cases, antibodies or fragments thereof may include an amino acid sequence with from about 50% to about 99.9% sequence identity (e.g. from about 50% to about 60%, from about 55% to about 65%, from about 60% to about 70%, from about 65% to about 75%, from about 70% to about 80%, from about 75% to about 85%, from about 80% to about 90%, from about 85% to about 95%, from about 90% to about 99.9%, from about 95% to about 99.9%, about 97%, about 97.5%, about 98%, about 98.5%, about 99%, about 99.5%, about 99.6%, about 99.7% or about 99.8%) with one or more of the constant domain sequences listed in Table 3. In some cases, antibodies or fragments thereof of the invention may include an amino acid sequence having one or more fragments of any of the sequences listed in Table 3.

TABLE 3
IgG Constant domain sequences

| | | SEQ |
|---|---|---|
| | | ID |
| Domain | Sequence | NO |
| Human | ASTKGPSVFPLAPSSKSTSGGTAALGCLVKDYFPEPVTVS | 3 |
| IgG1 | WNSGALTSGVHTFPAVLQSSGLYSLSSVVTVPSSSLGTQT | |
| heavy | YICNVNHKPSNTKVDKKVEPKSCDKTHTCPPCPAPELLGG | |
| chain | PSVFLFPPKPKDTLMISRTPEVTCVVVDVSHEDPEVKFNW | |
| constant | YVDGVEVHNAKTKPREEQYNSTYRVVSVLTVLHQDWLN | |
| regions | GKEYKCKVSNKALPAPIEKTISKAKGQPREPQVYTLPPSRD | |
| | ELTKNQVSLTCLVKGFYPSDIAVEWESNGQPENNYKTTPP | |
| | VLDSDGSFFLYSKLTVDKSRWQQGNVFSCSVMHEALHNH | |
| | YTQKSLSLSPGK | |
| Human | RTVAAPSVFIFPPSDEQLKSGTASVVCLLNNFYPREAKVQ | 4 |
| IgG1 light | WKVDNALQSGNSQESVTEQDSKDSTYSLSSTLTLSKADYE | |
| chain | KHKVYACEVTHQGLSSPVTKSFNRGEC | |
| constant | | |
| regions | | |

### Humanized Antibodies

“Humanized” forms of non-human (e.g., murine) antibodies are chimeric antibodies that contain minimal sequences derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

In some embodiments, fully humanized heavy and light chains may be designed from antibody sequences and/or with CDRs presented herein. Protein models of antibody variable regions may be generated using existing antibody structures as templates. Segments of starting heavy and light chain variable region amino acid sequences may be compared with human sequences to identify human germline antibodies with similar sequences. Series of humanized heavy and light chain variable regions may be designed using human variable domain framework region sequences with the objective that T cell epitopes be avoided. Variant human sequence segments with significant incidence of potential T cell epitopes as determined by in silico technologies may then be discarded. In some cases, some of the amino acid residues in resulting variable domains may be mutated back to amino acids present in the original mouse variable domain. In some cases, some of the mouse residues in the resulting variable domains may be mutated to match residues present in human germline sequences.

Humanized heavy and light chain variable region genes may be constructed from overlapping oligonucleotides assembled into full length genes using the ligase chain reaction (LCR). LCR products may be amplified and suitable restriction sites may be added for cloning into expression vectors. PCR products may be cloned into intermediate vectors and confirmed by sequencing.

For construction of expression plasmids encoding fully humanized antibodies with human constant regions, DNA sequences encoding antibody variable region may be inserted into expression vectors (e.g., mammalian expression vectors) between an upstream promoter/enhancer, for example, cytomegalovirus immediate/early promoter/enhancer (CMV IE), plus the immunoglobulin signal sequence and a downstream immunoglobulin constant region gene. DNA samples may then be prepared for transfection into mammalian cells.

For generation of cell lines and selection of fully humanized antibodies, heavy and light chain plasmid DNA pairs may be transfected into cells for expression. In some embodiments, mammalian NS0 cells may be used. Cell lines producing humanized antibodies may be expanded for expression antibodies that may be harvested and purified from cell culture media.

In some embodiments, antibodies of the present disclosure may be prepared according to humanization methods known in the art. Such methods may include, but are not limited to CDR grafting, resurfacing, superhumanization, and human string content optimization (see, for example, Almagro, et al., 2008. Front. Biosci. 13:1619-33). In some embodiments, empirical methods are used. Such methods may include the generation of large combinatorial libraries and selecting desired variants by enrichment technologies, such as phage display, yeast display, ribosomal display, or other high throughput screening techniques. These methods may be utilized alone or in combination with framework libraries, guided selection, framework shuffling, and humaneering.

### Antibody Sequence Optimization

Variable domain sequences may be analyzed for sequence characteristics that may impact antibody function, expression, stability, and/or immunogenicity. In some cases, such characteristics may include NG residue pairs. NG residue pairs may be susceptible to asparagine deamidation, with possible conversion to glutamate and pyroglutamate in a 3:1 ratio over time. These residue pairs may be mutated, for example, to SG or QG pairs to prevent deamidation at these sites. Alternatively, these antibodies may be formulated to reduce deamidation.

In some embodiments, aspartate isomerization sites may be identified and altered. Aspartate isomerization sites include DG amino acid residue pairs. Aspartic acid at these sites can convert to glutamate and pyroglutamate in a 3:1 ratio overtime. DG residue pairs may be mutated to SG or QG residue pairs to prevent isomerization at these sites. Alternatively, these antibodies may be formulated to reduce deamidation.

In some embodiments, N-terminal glutamine residues may be converted to N-terminal glutamate residues. This may prevent N-terminal pyrrolization.

In some embodiments, one or more aggregation-prone patch of amino acid residues may be mutated. These may include patches having amino acids with bulky side chains, for example, histidine, phenylalanine, and tryptophan.

In some embodiments, one or more cysteine residues may be mutated to prevent the presence of unpaired cysteines. Unpaired cysteines may be reactive, for example, when accessible to solvent as part of an antibody. In some cases, unpaired cysteine residues may be mutated to serine.

In some embodiments, one or more glycosylation sites (e.g., N-linked NXS/T sites), acid cleavage sites, and amino acid oxidation sites are mutated to improve antibody production, stability, binding, and/or activity.

### IgG Synthesis

IgG antibodies (e.g. IgG1, IgG2, IgG3 or IgG4) including one or more variable domain and/or CDR amino acid sequences presented herein (or fragment or variants thereof) may be synthesized for further testing and/or product development. Such antibodies may be produced by insertion of one or more segments of cDNA encoding desired amino acid sequences into expression vectors suited for IgG production. Expression vectors may include mammalian expression vectors suitable for IgG expression in mammalian cells. Mammalian expression of IgGs may be carried out to ensure that antibodies produced include modifications (e.g. glycosylation) characteristic of mammalian proteins and/or to ensure that antibody preparations lack endotoxin and/or other contaminants that may be present in protein preparations from bacterial expression systems.

### Antibody Sequence and Structural Analysis and Optimization

In some embodiments, antibodies of the present invention may be subjected to sequence analysis and/or structural analysis wherein they are analyzed for characteristics that may affect antibody chemistry, affinity, specificity, protein folding, stability, manufacturing, expression, and/or immunogenicity (i.e., immune reactions in subjects being treated with such antibodies). Such analysis may include comparisons between antibodies binding to the same or similar epitopes.

Antibodies sequences of antibodies binding to the same epitope may be analyzed for variation in light and/or heavy chain sequences. Such analysis may include germline sequences and/or CDR sequences. Information obtained from such analysis may be used to identify (and optionally to modify, delete, replace or repair) conserved amino acid residues; conserved segments of amino acids; amino acid positions with conserved side chain characteristics; conserved CDR lengths; and other features conserved among antibodies binding to the same epitope. This information may be used to design variants or to inform antibody optimization procedures to improve antibody affinity, specificity, protein folding, stability, manufacturing, expression and/or immunogenicity.

Sequence analysis may include aligning two or more antibodies that bind to the same or similar epitopes to identify similarities. Such analysis may compare the sequence and/or length of antibody regions (e.g., CDRs, variable domains, germline segments). Amino acid insertions, amino acid deletions, and substitutions may be identified and assessed. Sequence differences may be compared against antibody affinity and/or specificity.

In some cases, sequence analyses are conducted to identify (and optionally to modify, delete, replace or repair) one or more of unpaired cysteines or irregular disulfides; glycosylation sites (e.g., N-linked NXS/T sites); acid cleavage sites, amino acid oxidation sites, conformity with mouse germline sequences; asparagine deamidation sites; aspartate isomerization sites; N-terminal pyroglutamate formation sites; and aggregation-prone patches in CDRs.

In some cases, the present invention provides sequence analysis-informed variants of antibodies presented herein. As used herein, the term “sequence analysis-informed variant” refers to an antibody variant that has been modified based on one or more conclusions derived from antibody sequence analysis. In some cases, antibodies of the invention may be modified to produce antibody variants that include modifications to one or more of antibody affinity, specificity, protein folding, stability, manufacturing, expression and/or immunogenicity.

Some sequence analysis-informed variants include one or more CDR length modification. CDR length modified antibodies may include one or more added or deleted amino acids in one or more CDRs relative to an original antibody sequence. In some cases, sequence analysis-informed variants may include a substitution of one or more CDRs with one or more CDRs derived from another antibody (e.g., an antibody binding to the same or similar epitope). In some cases, sequence analysis-informed variants may include a substitution of a heavy or light chain variable domain from another antibody (e.g., an antibody binding to the same or similar epitope). Sequence analysis-informed variants may include modifications to one or more germline genes that the antibody is expressed from. Such modifications may include point mutations, regional mutations, insertional mutations or deletional mutations. In some case, germline gene modifications are carried out to move CDRs from one known germline gene to another. Sequence analysis-informed variants may include other variants described herein, including, but not limited to scFvs, monobodies, diabodies, intrabodies, CARs, antibody mimetics, etc.

In some embodiments, sequence and/or structural analysis may be used to inform the construction of antibody fragment display libraries (including, but not limited to scFv libraries, phage display libraries, and yeast display libraries). In one example, sequence alignment may be carried out to align two or more antibodies with a common antigen or epitope and amino acid residues may be identified that are conserved among the aligned antibodies or that are variable among the aligned antibodies. In such cases, antibody fragment display libraries may be constructed such that variability among library members is primarily limited to the variable amino acids identified in the sequence analysis. In some cases, such libraries may be used to identify variants with altered affinity and/or specificity for a target antigen (e.g., STn) or a specific epitope of the target antigen (e.g., the epitopes recognized by Group 1, 2, 3 and 4 antibodies as described in Example 1, hereinbelow).

In some embodiments, antibodies of the invention may be modified to remove, replace or otherwise eliminate one or more unpaired cysteine residues. In some cases, unpaired cysteine residues may be reactive and may affect antibody affinity and/or specificity. Accordingly, some antibodies of the invention have been modified to eliminate unpaired cysteine residues. In some cases, such variants may have modified epitope specificity and/or affinity. In some cases, modification of unpaired cysteine residues may alter antibody folding. In some cases, these variants include a substitution or deletion of one or more cysteine residues. In some cases, these variants include one or more additional amino acid residues (including, but not limited to, the addition of one or more cysteine residues) to prevent or reduce undesired effects from unpaired cysteine residues. In some cases, cysteine residues are replaced with an amino acid having a hydrophobic side chain (e.g., tyrosine, alanine, valine, isoleucine, leucine, methionine, phenylalanine or tryptophan).

### Antibody Testing and Characterization

Antibodies described herein may be tested and/or characterized using a variety of methods. Such methods may be used to determine a variety of characteristics that may include, but are not limited to, antibody affinity; specificity; and activity (e.g., activation or inhibition of cellular signaling pathways or other cellular or biological activities). Antibody testing may further include testing in vivo (e.g., in animal and/or human studies) for one or more of toxicity, therapeutic effect, pharmacodynamics, pharmacokinetics, absorption, deposition, metabolism, and excretion. Testing in animals may include, but is not limited to, testing in mice, rats, rabbits, guinea pigs, pigs, primates (e.g., Cynomolgus monkeys), sheep, goats, horses, and cattle.

### Cell-Based Assays

In some embodiments, antibodies of the present invention may be tested or characterized through the use of one or more cell-based assays. Such cell-based assays may be carried out in vitro with cells in culture. In some cases, cell-based assays may be carried out in vivo. Examples of cell-based in vivo assays include tumor models in which tumor cells are injected or otherwise introduced into a host.

In some cases, cells used in cell-based assays may express one or more target glycans recognized by one or more antibodies of the invention. Such glycans may be naturally expressed by such cells or, alternatively, cells may be induced to express one or more glycans desired for purposes of a particular assay. Induced expression may be through one or more treatments that upregulate expression of glycosylated proteins or enzymes that regulate glycosylation. In other cases, induced expression may include transfection, transduction, or other form of introduction of one or more genes or transcripts for the endogenous expression of one or more glycosylated proteins or enzymes involved in regulation of glycosylation.

In some cases, cell-based assays used herein may include the use of cancer cells. Many cancer cell lines are available for experiments to test antibodies of the invention. Such cells may express target glycan or may be induced to express target glycans. Additionally, cancer cell lines may be used to test antibodies of the invention, where the cancer cell lines are representative of cancer stem cells. Cancer stem cell (CSC) cell lines may be isolated or differentiated from cancer cells grown in culture (e.g., through sorting based on markers specific for cancer stem cells). Cell lines used in cell-based assays may include, but are not limited to breast, colon, ovary, lymphocyte, bone marrow, and skin cell lines. Specific cell lines may include, but are not limited to SNU-16 cells, LS-174T cells, MC38 cells, TOV-112D cells, TOV-21G cells, Jurkat E6.1 cells, K-562 cells, B16-F0 cells, B16-F10 cells, LS180 cells, COLO205 cells, TB4 cells, HT29 cells, Panc1 cells, HPAC cells, HPAFII cells, RKO cells, SW480 cells, and SNU-C2A cells.

In some embodiments, ovarian cancer cell lines may be used. Such cell lines may include, but are not limited to SKOV3, OVCAR3, OV90 and A2870 cell lines. In some cases, CSC cells may be isolated from these cell lines by isolating cells expressing CD44 and/or CD133 cell markers.

OVCAR3 cells were first established using malignant ascites obtained from a patient suffering from progressive ovarian adenocarcinoma (Hamilton, T. C. et al., 1983. Cancer Res. 43: 5379-89). Cancer stem cell populations may be isolated from OVCAR3 cell cultures through selection based on specific cell surface markers such as CD44 (involved in cell adhesion and migration), CD133 and CD117 (Liang, D. et al., 2012. BMC Cancer. 12: 201, the contents of which are herein incorporated by reference in their entirety). OV90 cells are epithelial ovarian cancer cells that were similarly derived from human ascites (see U.S. Pat. No. 5,710,038). OV-90 cells may also express CD44 when activated (Meunier, L. et al., 2010. Transl Oncol. 3(4): 230-8).

In some embodiments, cell lines derived from gastric cancers may be used. Such cell lines may include, but are not limited to SNU-16 cells (see description in Park J. G. et al., 1990. Cancer Res. 50: 2773-80, the contents of which are herein incorporated by reference in their entirety). SNU-16 cells express STn naturally, but at low levels.

In some embodiments, methods of the present disclosure include methods of characterizing glycan-interacting antibodies by contacting colorectal cells with glycan-interacting antibodies and evaluating antibody binding to the cells, antibody internalization into the cells, and/or antibody killing of the cells. According to some such methods, the colorectal cells may be derived from a colorectal cell line grown in vitro (e.g., propagated through cell culture). In some cases, colorectal cell lines are derived from a tumor. In other embodiments, colorectal cell lines may be derived from a tumor formed using a xenograft animal model (e.g., a xenograft mouse model). Colorectal cells used for characterizing glycan-interacting antibodies may be from a patient (e.g., a patient tumor). Methods of characterizing glycan-interacting antibodies may include the use of tissue micro arrays, including those having one or more colorectal cells.

Characterizing glycan-interacting antibodies with colorectal cells may include evaluating binding between such antibodies and cells by determining the EC50 of binding of the glycan-interacting antibody to the colorectal cell. The EC₅₀ may be determined by using one or more of flow cytometry analysis and ELISA analysis. In some embodiments, characterizing glycan-interacting antibodies with colorectal cells may include evaluating the killing of such cells by glycan-interacting antibodies. This may be carried out by treating colorectal cells with glycan-interacting antibodies and using a cell viability assay to determine the percentage of cells killed by the treatment. In some cases, evaluating killing of colorectal cells by glycan-interacting antibodies includes determining the IC₅₀ for glycan-interacting antibody killing of colorectal cells. In some cases, the antibodies may be conjugated with a cytotoxic agent (e.g., MMAE or MMAF).

### Glycan Arrays

In some embodiments, glycan-interacting antibodies of the present invention may be developed through the use of glycan arrays. As used herein, the term “glycan array” refers to a tool used to identify agents that interact with any of a number of different glycans linked to the array substrate. In some embodiments, glycan arrays include a number of chemically-synthesized glycans, referred to herein as “glycan probes”. In some embodiments, glycan arrays include at least 2, at least 5, at least 10, at least 20, at least 30, at least 40, at least 50, at least 60, at least 70, at least 80, at least 90, at least 100, at least 150, at least 350, at least 1000 or at least 1500 glycan probes. In some embodiments, glycan arrays may be customized to present a desired set of glycan probes. In some embodiments, glycan probes may be attached to the array substrate by a linker molecule. Such linkers may include molecules including, but not limited to —O(CH₂)₂CH₂)NH₂ and O(CH₂)₃NHCOCH₂(OCH₂CH₂)₆NH₂.

In some embodiments, a glycan array has more than 70 chemically-synthesized glycans, most of which are presented as Neu5Ac and Neu5Gc-containing glycan pairs. Some examples of glycan probes may include: Neu5Ac-α-2-6-GalNAc (AcSTn); Neu5Gc-α-2-6-GalNAc (GcSTn); Neu5,9Ac2-α-2,6-GalNAc; Neu9Ac5Gc-α-2,6-GalNAc, and GalNAc (Tn). The antibody binding specificity to AcSTn vs. GcSTn can be determined using the array or other methods of determining specificity known in the art. In addition, the binding profile of antibodies to O-acetylated STn can be determined. The loss of O-acetylation on STn is relevant to cancer as cancer-associated expression correlates with increased STn recognition by antibodies (Ogata, S. et al., Tumor-associated sialylated antigens are constitutively expressed in normal human colonic mucosa. Cancer Res. 1995 May 1; 55(9):1869-74) In some cases, glycan arrays may be used to determine recognition of STn vs. Tn.

### Antibody Fragment Display Library Screening Techniques

In some embodiments, antibodies of the present invention may be produced and/or optimized using high throughput methods of discovery. Such methods may include any of the display techniques (e.g. display library screening techniques) disclosed in International Patent Application No. WO2014074532, the contents of which are herein incorporated by reference in their entirety. In some embodiments, synthetic antibodies may be designed, selected or optimized by screening target antigens using display technologies (e.g. phage display technologies). Phage display libraries may include millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries include scFv antibody fragments that include a fusion protein of V_{H} and V_{L} antibody domains joined by a flexible linker. In some cases, scFvs may contain the same sequence with the exception of unique sequences encoding variable loops of the complementarity determining regions (CDRs). In some cases, scFvs are expressed as fusion proteins, linked to viral coat proteins (e.g. the N-terminus of the viral pIII coat protein). V_{L} chains may be expressed separately for assembly with V_{H} chains in the periplasm prior to complex incorporation into viral coats. Precipitated library members may be sequenced from the bound phage to obtain cDNA encoding desired scFvs. Such sequences may be directly incorporated into antibody sequences for recombinant antibody production, or mutated and utilized for further optimization through in vitro affinity maturation.

### Development of Cytotoxic Antibodies

In some embodiments, antibodies of the present invention may be capable of inducing antibody-dependent cell-mediated cytotoxicity (ADCC) and/or antibody-dependent cell phagocytosis (ADCP). ADCC is an immune mechanism whereby cells are lysed as a result of immune cell attack. Such immune cells may include CD56+ cells, CD3− natural killer (NK) cells, monocytes and neutrophils (Strohl, W. R. Therapeutic Antibody Engineering. Woodhead Publishing, Philadelphia PA. 2012. Ch. 8, p 186, the contents of which are herein incorporated by reference in their entirety).

In some cases, antibodies of the present invention may be engineered to include a given isotype depending on whether or not ADCC or ADCP is desired upon antibody binding. Such antibodies, for example, may be engineered according to any of the methods disclosed by Alderson, K. L. et al., J Biomed Biotechnol. 2011. 2011:379123). In the case of mouse antibodies, different isotypes of antibodies are more effective at promoting ADCC. IgG2a, for example, is more effective at inducing ADCC than is IgG2b. Some antibodies of the present invention, including mouse IgG2b antibodies may be reengineered to be IgG2a antibodies. Such reengineered antibodies may be more effective at inducing ADCC upon binding cell-associated antigens. In some embodiments, antibodies are reengineered by modifying or introducing one or more post-translational modifications to improve ADCC and/or CDC biological activity.

In some embodiments, genes encoding variable regions of antibodies developed according to methods of the present invention may be cloned into mammalian expression vectors encoding human Fc regions. Such Fc regions may be Fc regions from human IgG1κ. IgG1κ Fc regions may include amino acid mutations known to enhance Fc-receptor binding and ADCC activity.

### Antibody-Drug Conjugates

In some embodiments, antibodies of the invention may be developed for antibody-drug conjugate (ADC) therapeutic applications. ADCs are antibodies in which one or more cargo (e.g., therapeutic agents) are attached [e.g. directly or via linker (e.g. a cleavable linker or a non-cleavable linker)]. ADCs are useful for delivery of therapeutic agents (e.g., drugs or cytotoxic agents) to one or more target cells or tissues (Panowski, S. et al., 2014. mAbs 6:1, 34-45). In some cases, ADCs may be designed to bind to a surface antigen on a targeted cell. Upon binding, the entire antibody-antigen complex may be internalized and directed to a cellular lysosome. ADCs may then be degraded, releasing the bound cargo. Where the cargo is a cytotoxic agent, the target cell will be killed or otherwise disabled. Cytotoxic agents may include, but are not limited to cytoskeletal inhibitors [e.g., tubulin polymerization inhibitors, and kinesin spindle protein (KSP) inhibitors], DNA damaging agents (e.g., calicheamicins, duocarmycins, and pyrrolobenzodiazepine dimers such as talirine and tesirine), topoisomerase inhibitors [e.g., camptothecin compounds or derivatives such as 7-ethyl-10-hydroxycamptothecin (SN-38) and exatecan derivative DXd], transcription inhibitors (e.g., RNA polymerase inhibitors such as amanitin), and kinase inhibitors [e.g., phosphoinositide 3-kinase (PI3K) inhibitors or mitogen-activated protein kinase kinase (MEK) inhibitors].

Tubulin polymerization inhibitors may include, but are not limited to, maytansines (e.g., emtansine [DM1] and ravtansine [DM4]), auristatins, tubulysins, and vinca alkaloids or derivatives thereof. Exemplary auristatins include auristatin E (also known as a derivative of dolastatin-10), auristatin EB (AEB), auristatin EFP (AEFP), monomethyl auristatin E (MMAE), monomethyl auristatin F (MMAF), auristatin F and dolastatin. Exemplary tubulysin compounds include naturally occurring tubulysins A, B, C, D, E, F, G, H, I, U, and V, and tubulysin analogs such as pretubulysin D (PTb-D43) and N¹⁴-desacetoxytubulysin H (Tb1). Exemplary vinca alkaloids include vincristine, vinblastine, vindesine, and navelbine (vinorelbine). In some embodiments, cytotoxic agents may include auristatin derivatives [e.g. 1-aminopropan-2-yl-auristatin F, auristatin F-hydroxypropylamide, auristatin F-propylamide, auristatin F phenylenediamine (AFP)]; tubulysin derivatives; vinca alkaloid derivatives [e.g. N-(3-hydroxypropyl)vindesine (HPV)], and any of those described in U.S. Pat. Nos. 8,524,214; 8,685,383; 8,808,679; and 9,254,339; US Patent Application Publications US20150314008A1, US20160220696A1 and US20160022829A1; the contents of each of which are herein incorporated by reference in their entirety.

In some embodiments, antibody-drug conjugates (ADCs) of the invention may further comprise one or more polymeric carrier connecting the antibody and the therapeutic agents (e.g., antibody-polymer-drug conjugates). As used herein, the term “polymeric carrier” refers to a polymer or a modified polymer, which may be covalently attached to one or more therapeutic agents and/or antibodies. Polymeric carriers may provide additional conjugation sites for therapeutic agents, increasing the drug-to-antibody ratio and enhancing therapeutic effects of ADCs. In some embodiments, polymeric carriers used in this invention may be water soluble and/or biodegradable. Such polymeric carriers may include, but are not limited to poly(ethylene glycol) (PEG), poly(N-(2-hydroxypropyl)methacrylamide) (polyHPMA), poly(α-amino acids) [e.g., poly(L-lysine), poly(L-glutamic acid), and poly((N-hydroxyalkyl)glutamine)], carbohydrate polymers [e.g., dextrins, hydroxyethylstarch (HES), and polysialic acid], glycopolysaccharides (e.g., homopolysaccharide such as cellulose, amylose, dextran, levan, fucoidan, carraginan, inulin, pectin, amylopectin, glycogen and lixenan; or homopolysaccharide such as agarose, hyluronan, chondroitinsulfate, dermatansulfate, keratansulfate, alginic acid and heparin), glycolipids, glycoconjugates, polyglycerols, polyvinyl alcohols, poly(acrylic acid), polyketal and polyacetal [e.g., poly(1-hydroxymethylethylene hydroxymethylformal), also known as PHF or FLEXIMER®, described in U.S. Pat. Nos. 5,811,510; 5,863,990; and 5,958,398; the contents of each of which are herein incorporated by reference in their entirety], and derivatives, dendrimers, copolymers and mixtures thereof. For example, the polymeric carrier may include a copolymer of a polyacetal/polyketal (e.g., PHF) and a hydrophilic polymer such as polyacrylates, polyvinyl polymers, polyesters, polyorthoesters, polyamides, polypeptides, and derivatives thereof.

In some embodiments, therapeutic agents are attached (e.g., covalently bonded) to antibodies of the invention directly or via linkers. In some embodiments, therapeutic agents are attached to polymeric carriers directly or via linkers, and the polymeric carriers are attached to the antibodies directly or via linkers. In some embodiments, linkers may comprise an oxalic, malonic, succinic, glutaric, adipic, pimelic, suberic, azelaic, sebacic, phthalic, isophthalic, terephthalic, diglycolic acid, tartaric, glutamic, fumaric, or aspartic moiety, including amide, imide, or cyclic-imide derivatives of each thereof, and each optionally substituted. Exemplary linkers may include any of those disclosed in U.S. Pat. Nos. 8,524,214; 8,685,383; 8,808,679; 9,254,339; and/or 9,555,112 the contents of each of which are herein incorporated by reference in their entirety.

In some embodiments, linkers may be cleavable linkers. Cleavable linkers may break down under certain conditions (such as changes in pH, temperature, or reduction) or cleaved by enzymes (e.g., proteases and glucuronidases) to allow release of therapeutic agents from ADCs. Such linkers may include a labile bond such as an ester bond, amide bond, or disulfide bond. Non-limiting cleavable linkers may include pH-sensitive linkers (e.g., hydrazone, semicarbazone, thiosemicarbazone, cis-aconitic amide, thioether, orthoester, acetal, or ketal); reduction-sensitive linkers [e.g., N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), N-succinimidyl-S-acetylthioacetate (SATA) and N-succinimidyl-oxycarbonyl-alpha-methyl-alpha-(2-pyridyl-dithio)toluene or 2,5-dioxopyrrolidin-1-yl 4-(1-(pyridin-2-yldisulfanyl)ethyl)benzoate (SMPT)]; photosensitive linkers; and enzymatically cleavable linkers [e.g., peptide linkers such as valine-citrulline, valine-citrulline-p-aminobenzoyloxycarbonyl (vc-PAB), maleimidocaproyl-valine-citrulline-p-aminobenzoyloxycarbonyl (MC-vc-PAB), linkers cleavable by glucuronidases, such as glucuronide-MABC, or linkers cleavable by esterases].

In other embodiments, linkers may be non-cleavable linkers. Non-cleavable linkers may increase plasma stability of the ADCs compared to cleavable linkers. Exemplary non-cleavable linkers include maleimide alkane and maleimide cyclohexane (MCC).

Antibody-drug conjugates (ADCs) of the invention may be prepared using any method known in the art. For example, therapeutic agents may be modified to contain a functional group that can react with a functional group on the antibody. Antibody-drug conjugates (ADCs) may be prepared by reacting the two functional groups to form a conjugate. In some cases, polymeric carriers may be modified to contain functional groups that can react with the functional group on the therapeutic agents and the functional group on the antibody under different chemical conditions. Antibodies, polymeric carriers, and therapeutic agents may be linked to form the antibody-polymer-drug conjugates through sequential chemical reactions. Conjugation to antibodies may employ a lysine or a cysteine residue as the conjugation site. In some embodiments, antibodies may be engineered to have additional lysine or cysteine residues. Such approaches may avoid disruption of antibody structure (e.g., interchain disulfide bonds) and maintain antibody stability and/or activity.

As described herein, drug-to-antibody ratio (DAR) is the average number of therapeutic agents (e.g., drugs or cytotoxic agents) conjugated to the antibodies. In some embodiments, drug-to-antibody ratio of an ADC of the invention is at least 1:1, at least 2:1, at least 4:1, at least 6:1, at least 8:1, at least 10:1, at least 12:1, at least 15:1, at least 20:1 or at least 25:1.

In some embodiments, antibodies of the invention may be tested for their ability to promote cell death when developed as ADCs. Cell viability assays may be performed in the presence and absence of secondary antibody-drug conjugates. Antibodies with potent cell growth inhibition may then be used to design direct antibody-drug conjugates (ADCs). The use of such secondary antibody-drug conjugates in cell-based cytotoxic assays may allow for quick pre-screening of many ADC candidates. Based on such assays, an unconjugated antibody candidate is directly added to cells in the presence of a secondary antibody that is conjugated to one or more cytotoxic agents (referred to herein as a 2° ADC). Internalization of the antibody/2° ADC complex into cells that express a high density of the targeted antigen can achieve a dose-dependent drug release within the cells, causing a cytotoxic effect to kill the cells (e.g., tumor cells), while cells expressing a low density of the targeted antigen are not affected (e.g., normal cells).

ADCs of the invention may be designed to target cancer cells. Such ADCs may include antibodies directed to one or more tumor-associated carbohydrate antigen (TACA). In some cases, ADCs of the invention are anti-STn antibodies.

### Development of Chimeric Antigen Receptors

In some embodiments, antibody sequences of the invention may be used to develop a chimeric antigen receptor (CAR). CARs are transmembrane receptors expressed on immune cells that facilitate recognition and killing of target cells (e.g. tumor cells). CARs typically include three basic parts. These include an ectodomain (also known as the recognition domain), a transmembrane domain and an intracellular (signaling) domain. Ectodomains facilitate binding to cellular antigens on target cells, while intracellular domains typically include cell signaling functions to promote the killing of bound target cells. Further, they may have an extracellular domain with one or more antibody variable domains described herein or fragments thereof. CARs of the invention also include a transmembrane domain and cytoplasmic tail. CARs may be designed to include one or more segments of an antibody, antibody variable domain and/or antibody CDR, such that when such CARs are expressed on immune effector cells, the immune effector cells bind and clear any cells that are recognized by the antibody portions of the CARs.

Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

CARs engineered to target tumors may have specificity for one or more tumor associated carbohydrate antigens (TACAs). In some embodiments, ectodomains of these CARs may include one or more antibody variable domains or a fragment thereof. In some embodiments, CARs are expressed in T cells, and may be referred to as “CAR-engineered T cells” or “CAR-Ts”. CAR-Ts may be engineered with CAR ectodomains having one or more antibody variable domains.

### Structural Features of Chimeric Antigen Receptors

With gene-transfer technology, T cells can be engineered to stably express antibodies on their surface, conferring a desired antigen specificity. Chimeric antigen receptors (CARs) combine an antigen-recognition domain of a specific antibody with an intracellular domain of the CD3-zeta chain or FcγRI protein having T cell activating properties into a single chimeric fusion protein. CAR technology provides MHC-unrestricted recognition of target cells by T cells. Removal of the MHC restriction of T cells facilitates the use of these molecules in any patient, and also, in both CD8⁺ and CD4⁺ T cells, usually restricted to MHC class I or II epitopes, respectively. The use of Ab-binding regions allows T cells to respond to epitopes formed not only by protein, but also carbohydrate and lipid. This chimeric receptor approach is especially suited to immunotherapy of cancer, being able to bypass many of the mechanisms by which tumors avoid immunorecognition, such as MHC down-regulation, lack of expression of costimulatory molecules, CTL resistance, and induction of T cell suppression, and where the use of both CD8⁺ CTL and CD4⁺ T cells are best combined for optimum antitumor efficacy. This approach has been demonstrated to be applicable to a wide range of tumor antigens, in addition to viruses such as HIV (Finney, et al., J. Immunology, 2004, 172:104-113).

Although chimeric antigen receptors can trigger T-cell activation in a manner similar to that of endogenous T-cell receptors, in practice, the clinical application of CAR technology has been impeded by inadequate in vivo expansion of chimeric antigen receptor T cells. For example, first generation CARs included as their signaling domain the cytoplasmic region of the CD3ζ or Fc receptor γ chain. These first-generation CARs were tested in phase I clinical studies in patients with ovarian cancer, renal cancer, lymphoma, and neuroblastoma, and were found to induce modest responses, effectively redirecting T cell cytotoxicity but failing to enable T cell proliferation and survival upon repeated antigen exposure. The prototypes for second generation CARs involved receptors encompassing both CD28 and CD3ζ, and second generation CARs have been tested for treatment of B cell malignancies and other cancers (Sadelain, et al., (2009) Current Opinion in Immunology, 21(2):215-223). Thus, CARs have rapidly expanded into a diverse array of receptors with different functional properties.

More recently, it was discovered that CAR-mediated T-cell responses can be enhanced with the addition of a costimulatory domain. In preclinical models, the inclusion of the CD137 (4-1BB) signaling domain was found to significantly increase antitumor activity and in vivo persistence of chimeric antigen receptors as compared with inclusion of the CD3-zeta chain alone (Porter, et al., N. Engl. J. Med. 2011, 365:725-733).

Thus, in some embodiments of the present disclosure, antibody sequences of the invention may be used to develop a chimeric antigen receptor (CAR). In some embodiments, CARs are transmembrane receptors expressed on immune cells that facilitate recognition and killing of target cells (e.g. tumor cells).

In many cancers, tumor-specific antigens for targeting have not been defined, but in B-cell neoplasms, CD19 is an attractive target. Expression of CD19 is restricted to normal and malignant B cells and B-cell precursors. A pilot clinical trial of treatment with autologous T cells expressing an anti-CD19 chimeric antigen receptor (CART19) was performed in patients with advanced, p53-deficient chronic lymphoid leukemia (CLL). The generation of a CD19-specific immune response in bone marrow was demonstrated by temporal release of cytokines and ablation of leukemia cells that coincided with peak infiltration of chimeric antigen receptor T cells. (Porter, et al., N. Engl. J. Med. 2011, 365:725-733).

Further structural features of CARs may include any of those disclosed in several PCT Publications assigned to City of Hope and having the common inventor Michael Jensen. For example, PCT Publication WO 00/23573 describes genetically engineered, CD20-specific redirected T cells expressing a cell surface protein having an extracellular domain that includes a receptor specific for CD20, an intracellular signaling domain, and a transmembrane domain. Use of such cells for cellular immunotherapy of CD20⁺ malignancies and for abrogating any untoward B cell function. In one embodiment, the cell surface protein is a single chain FvFc: ζ receptor where Fv designates the VH and VL chains of a single chain monoclonal antibody to CD20 linked by peptide, Fc represents a hinge-CH2-CH3 region of a human IgG1, and (represents the intracellular signaling domain of the zeta chain of human CD3. A method of making a redirected T cell expressing a chimeric T cell receptor by electroporation using naked DNA encoding the receptor. Similarly, PCT Publication WO 02/077029 describes genetically engineered, CD19-specific redirected immune cells expressing a cell surface protein having an extracellular domain that includes a receptor which is specific for CD19, an intracellular signaling domain, and a transmembrane domain. Use of such cells for cellular immunotherapy of CD19⁺ malignancies and for abrogating any untoward B cell function. In one embodiment, the immune cell is a T cell and the cell surface protein is a scFvFc:ζ receptor where scFv designates the VH and VL chains of a single chain monoclonal antibody to CD19, Fc represents at least part of a constant region of an IgG1, and zeta represents the intracellular signaling domain of the T cell antigen receptor complex zeta chain (zeta chain of human CD3). The extracellular domain scFvFc and the intracellular domain zeta are linked by a transmembrane domain such as the transmembrane domain of CD4. A method of making a redirected T cell expressing a chimeric T cell receptor by electroporation using naked DNA encoding the receptor. These chimeric antigen receptors have the ability, when expressed in T cells, to redirect antigen recognition based on the monoclonal antibody's specificity. The design of scFvFc:ζ receptors with target specificities for tumor cell-surface epitopes is a conceptually attractive strategy to generate antitumor immune effector cells for adoptive therapy as it does not rely on pre-existing anti-tumor immunity. These receptors are “universal” in that they bind antigen in a MHC independent fashion, thus, one receptor construct can be used to treat a population of patients with antigen positive tumors. City of Hope PCT Publications WO 02/088334, WO 2007/059298 and WO 2010/065818 describe “zetakines” made up of an extracellular domain that includes a soluble receptor ligand linked to a support region capable of tethering the extracellular domain to a cell surface, a transmembrane region and an intracellular signaling domain. Zetakines, when expressed on the surface of T lymphocytes, direct T cell activity to those specific cells expressing a receptor for which the soluble receptor ligand is specific.

Additional features of CARs may include any of those disclosed in two PCT Publications assigned to University of Texas and having a common inventor Lawrence Cooper. PCT Publication No. WO 2009/091826 describes compositions that include a human CD19-specific chimeric T cell receptor (or chimeric antigen receptor, CAR) polypeptide (designated hCD19CAR) that includes an intracellular signaling domain, a transmembrane domain and an extracellular domain, the extracellular domain including a human CD 19 binding region. In another aspect, the CD 19 binding region is an F(ab′)2, Fab′, Fab, Fv or scFv. The intracellular domain may include an intracellular signaling domain of human CD3ζ and may further include human CD28 intracellular segment. In certain aspects, the transmembrane domain is a CD28 transmembrane domain. PCT Publication No. WO 2013/074916 describes methods and compositions for immunotherapy employing CART T cells genetically modified to eliminate expression of T cell receptor and/or HLA. In particular embodiments, the T cell receptor-negative and/or HLA-negative T cells are generated using zinc finger nucleases, for example. The CAR⁺ T cells from allogeneic healthy donors can be administered to any patient without causing graft versus host disease (GVHD), acting as universal reagents for off-the-shelf treatment of medical conditions such as cancer, autoimmunity, and infection.

PCT Publication WO 2011/041093 assigned to the U.S. Department of Health and Human Services describes anti-vascular endothelial growth factor receptor-2 chimeric antigen receptors that include an antigen binding domain of a KDR-1121 or DC101 antibody, an extracellular hinge domain, a T cell receptor transmembrane domain, and an intracellular T cell receptor signaling domain, and their use in the treatment of cancer.

PCT Publications WO 2012/079000 and WO 2013/040557, the contents of each of which are herein incorporated by reference in their entirety, are assigned to University of Pennsylvania and share the common inventor Carl H. June; these publications describe CARS comprising an antigen binding domain, a transmembrane domain, a costimulatory signaling region, and a CD3 zeta signaling domain, and methods for generating RNA Chimeric Antigen Receptor (CAR) transfected T cells, respectively.

PCT Publication WO2013/126712, also assigned to University of Pennsylvania and sharing the common inventor Carl H. June, describes compositions and methods for generating a persisting population of T cells exhibiting prolonged exponential expansion in culture that is ligand independent and independent of the addition of exogenous cytokines or feeder cells, which are useful for the treatment of cancer. In some embodiments, the antigen binding domain is an anti-cMet binding domain. In some embodiments, the antigen binding domain is an anti-mesothelin binding domain. In some embodiments, the antigen binding domain is an anti-CD 19 binding domain. The hinge domain is IgG4, the transmembrane domain is a CD28 transmembrane domain. In some embodiments, the costimulatory signaling region is a CD28 signaling region. Also provided is a vector comprising a nucleic acid sequence encoding a chimeric antigen receptor (CAR), and the CAR comprising an antigen binding domain, a hinge domain, a transmembrane domain, a costimulatory signaling region, and a CD3 zeta signaling domain.

PCT Publication WO 2014/039513 assigned to University of Pennsylvania describes compositions and methods for inhibiting one or more diacylglycerol kinase (DGK) isoform in a cell in order to enhance the cytolytic activity of the cell. The cells may be used in adoptive T cell transfer in which, the cell is modified to express a CAR. Inhibition of DGK in T cells used in adoptive T cell transfer increases cytolytic activity of the T cells and thus may be used in the treatment of a variety of conditions, including cancer, infection, and immune disorders.

PCT Publication WO 2014/055771 assigned to University of Pennsylvania describes compositions and methods for treating ovarian cancer. Specifically, the invention relates to administering a genetically modified T cell having alpha-folate receptor (FR-alpha) binding domain and CD27 costimulatory domain to treat ovarian cancer. In one embodiment, the FR-alpha binding domain is said to be fully human, thereby preventing a host immune response.

In some embodiments, CARs of the invention may be engineered to target tumors. Such CARs may have specificity for one or more TACAs. In some case, ectodomains of these CARs may comprise one or more antibody variable domain presented herein or a fragment thereof. In some embodiments, CARs of the invention are expressed in T cells, referred to herein as “CAR-engineered T cells” or “CAR-Ts”. CAR-Ts may be engineered with CAR ectodomains having one or more antibody variable domain presented herein.

### Multispecific Antibodies

In some embodiments, antibodies of the present invention may bind more than one epitope. As used herein, the terms “multibody” or “multispecific antibody” refer to an antibody wherein two or more variable regions bind to different epitopes. The epitopes may be on the same or different targets. In certain embodiments, a multispecific antibody is a “bispecific antibody,” which recognizes two different epitopes on the same or different antigens.

### Bispecific Antibodies

Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. One common application for this technology is in cancer immunotherapy, where BsMAbs are engineered to simultaneously bind to a cytotoxic cell (using a receptor like CD3) and a target like a tumor cell to be destroyed.

Bispecific antibodies may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

A bispecific, single-chain antibody Fv fragment (Bs-scFv) was successfully used to kill cancer cells. Some human cancers are caused by functional defects in p53 that are restored by gene therapy with wild-type p53. Weisbart, et al., describe the construction and expression of a bispecific single-chain antibody that penetrates living colon cancer cells, binds intracellular p53, and targets and restores its wild type function (Weisbart, et al., Int. J. Oncol. 2004 October; 25(4):1113-8; and Weisbart, et al., Int. J. Oncol. 2004 December; 25(6):1867-73). In these studies, a bispecific, single-chain antibody Fv fragment (Bs-scFv) was constructed from (i) a single-chain Fv fragment of mAb 3E10 that penetrates living cells and localizes in the nucleus, and (ii) a single-chain Fv fragment of a non-penetrating antibody, mAb PAb421 that binds the C-terminal of p53. PAb421 binding restores wild-type functions of some p53 mutants, including those of SW480 human colon cancer cells. The Bs-scFv penetrated SW480 cells and was cytotoxic, suggesting an ability to restore activity to mutant p53. COS-7 cells (monkey kidney cells with wild-type p53) served as a control since they are unresponsive to PAb421 due to the presence of SV40 large T antigen that inhibits binding of PAb421 to p53. Bs-scFv penetrated COS-7 cells but was not cytotoxic, thereby eliminating non-specific toxicity of Bs-scFv unrelated to binding p53. Fv fragments alone were not cytotoxic, indicating that killing was due to transduction of p53. A single mutation in CDR1 of PAb421 VH eliminated binding of the Bs-scFv to p53 and abrogated cytotoxicity for SW480 cells without altering cellular penetration, further supporting the requirement of PAb421 binding to p53 for cytotoxicity (Weisbart, et al., Int. J. Oncol. 2004 October; 25(4):1113-8; and Weisbart, et al., Int. J. Oncol. 2004 December; 25(6):1867-73).

In some embodiments, antibodies of the present invention may be diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natd. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs. 2010. January-February; 2(1):77-83).

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications, and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs. 2010. January-February; 2(1):77-83).

Also included are maxibodies (bivalent scFv fused to the amino terminus of the Fc (CH2-CH3 domains) of IgG.

Bispecific T-cell-engager (BiTE) antibodies are designed to transiently engage cytotoxic T-cells for lysis of selected target cells. These typically include two scFvs (one binding to CD3 on T cells and one binding to a target antigen on the surface of a cell being targeted for destruction). In some embodiments, the two scFvs are joined by a linker. In other embodiments, the two scFvs are different regions on an antibody. The clinical activity of BiTE antibodies corroborates findings that ex vivo expanded, autologous T-cells derived from tumor tissue, or transfected with specific T-cell receptors, have shown therapeutic potential in the treatment of solid tumors. While these personalized approaches prove that T-cells alone can have considerable therapeutic activity, even in late-stage cancer, they are cumbersome to perform on a broad basis. This is different for cytotoxic T-lymphocyte antigen 4 (CTLA-4) antibodies, which facilitate generation of tumor-specific T-cell clones, and also for bi- and tri-specific antibodies that directly engage a large proportion of patients' T-cells for cancer cell lysis. The potential of global T-cell engagement for human cancer therapy by T-cell-engaging antibodies is under active investigation (Baeuerle P A, et al., Current Opinion in Molecular Therapeutics. 2009, 11(1):22-30 and Baeuerle P A and Reinhardt C, Cancer Res. 2009, 69(12): 4941-4, the contents of each of which are herein incorporated by reference in their entirety).

Third generation molecules include “miniaturized” antibodies. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE and possibly multiple sclerosis, although these projects remain in the earliest stages of clinical testing. (Nelson, A. L., MAbs. 2010. January-February; 2(1):77-83).

Genmab is researching application of their “Unibody” technology, in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and extended half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Biotecnol is also developing a “miniaturized” mAb, CAB051, which is a “compacted” 100 kDa anti-HER2 antibody in preclinical research (Nelson, A. L., MAbs. 2010. January-February; 2(1):77-83).

Recombinant therapeutics composed of single antigen-binding domains have also been developed, although they currently account for only 4% of the clinical pipeline. These molecules are extremely small, with molecular weights approximately one-tenth of those observed for full-sized mAbs. Arana and Domantis engineer molecules composed of antigen-binding domains of human immunoglobulin light or heavy chains, although only Arana has a candidate in clinical testing, ART-621, an anti-TNFα molecule in Phase 2 study for the treatment of psoriasis and rheumatoid arthritis. Ablynx produces “nanobodies” derived from the antigen-binding variable heavy chain regions (VHHS) of heavy chain antibodies found in camels and llamas, which lack light chains. Two Ablynx anti-von Willebrand Factor nanobodies have advanced to clinical development, including ALX-0081, in Phase 2 development as an intravenous therapy to prevent thrombosis in patients undergoing percutaneous coronary intervention for acute coronary syndrome, and ALX-0681, a Phase 1 molecule for subcutaneous administration intended for both patients with acute coronary syndrome and thrombotic thrombocytopenic purpura (Nelson, A. L., MAbs. 2010. January-February; 2(1):77-83).

### Antibody-Coated Agents

In some embodiments, antibodies or antibody fragments described herein may be used to prepare a composition that includes an antibody-coated agent. As used herein, the term “antibody-coated agent” refers to any particle, nanoparticle, molecule, protein, fusion-protein, lipid, liposome, cell membrane, cell, or other structure that includes one or more surface-associated antibodies or antibody fragments. Antibody-coated agents may target one or more glycans, proteins, cells, tissues, and/or organs based on the specificity of the antibody or antibody fragments used for coating.

Antibody-coated agents may include associated, enclosed, or embedded cargo. The cargo may be a detectable label. Some cargo may include one or more therapeutic agent. Such therapeutic agents may include, but are not limited to drugs, chemotherapeutic agents, and cytotoxic agents. Cytotoxic agents may be used to kill or otherwise disable a cell. Cytotoxic agents may include, but are not limited to cytoskeletal inhibitors [e.g., tubulin polymerization inhibitors such as maytansines or auristatins (e.g. monomethyl auristatin E [MMAE] and monomethyl auristatin F [MMAF]), and kinesin spindle protein (KSP) inhibitors], DNA damaging agents (e.g., calicheamicins, duocarmycins, and pyrrolobenzodiazepine dimers such as talirine and tesirine), topoisomerase inhibitors [e.g., camptothecin compounds or derivatives such as 7-ethyl-10-hydroxycamptothecin (SN-38) and exatecan derivative DXd], transcription inhibitors (e.g., RNA polymerase inhibitors such as amanitin), and kinase inhibitors [e.g., phosphoinositide 3-kinase (PI3K) inhibitors or mitogen-activated protein kinase kinase (MEK) inhibitors].

In some embodiments, antibody-coated agents may include nanoparticles coated with one or more antibodies or antibody fragments described herein. Such antibody-coated agents may target one or more glycan, including, but not limited to cell-associated glycans. Some such antibody-coated agents include one or more cytotoxic agents.

### Proteins and Variants

Glycan-interacting antibodies of the present invention may exist as a whole polypeptide, a plurality of polypeptides or fragments of polypeptides, which independently may be encoded by one or more nucleic acids, a plurality of nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also include single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine. The amino acid sequences of the glycan-interacting antibodies of the invention may include naturally occurring amino acids and as such may be considered to be proteins, peptides, polypeptides, or fragments thereof.

Alternatively, the glycan-interacting antibodies may include both naturally and non-naturally occurring amino acids.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least 70%, at least 75%, at least 80%, at least 85%, at least 90%, at least 95%, at least 96%, at least 97%, at least 98%, at least 99%, at least 99.5% at least 99.8%, or at least 99.9% sequence identity as compared to a native sequence. “Sequence identity” as it applies to amino acid sequences or nucleotide sequences is defined as the percentage of residues in the candidate sequence that are identical with the residues in the second sequence after aligning the sequences and taking gaps and fragments into consideration, if necessary, to achieve the maximum percent sequence identity. Calculation of the percent identity of two polymeric sequences, for example, can be performed by aligning the two sequences for optimal comparison purposes (e.g., gaps can be introduced in one or both of a first and a second polymeric sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The residues at corresponding positions are then compared. When a position in the first sequence is occupied by the same residue as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The present invention contemplates several types of glycan-interacting antibodies which are amino acid based including variants and derivatives. These include substitutional, insertional, deletion and covalent variants and derivatives. As such, included within the scope of this invention are glycan-interacting antibody molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. Nos. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and includes four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to include an odd number of amino acids, a half-loop of the odd-numbered loop will include the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions.

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to include an odd number of amino acids, a half-domain of the odd-numbered domain will include the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids of any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain”. A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH₂)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Isotopic Variations

The glycan-interacting antibodies of the present invention may contain one or more atoms that are isotopes. As used herein, the term “isotope” refers to a chemical element that has one or more additional neutron. In one embodiment, compounds of the present invention may be deuterated. As used herein, the term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. The glycan-interacting antibodies may be deuterated in order to change a physical property of the compound, such as stability, or to allow the compounds to be used in diagnostic and experimental applications.

### Conjugates and Combinations

It is contemplated by the present invention that the glycan-interacting antibodies of the present invention may be complexed, conjugated or combined with one or more homologous or heterologous molecules. As used herein, “homologous molecule” means a molecule which is similar in at least one of structure or function relative to a starting molecule while a “heterologous molecule” is one that differs in at least one of structure or function relative to a starting molecule. Structural homologs are therefore molecules which are substantially structurally similar. They can be identical. Functional homologs are molecules which are substantially functionally similar. They can be identical.

Glycan-interacting antibodies of the invention may include conjugates. Such conjugates of the invention may include a naturally occurring substance or ligand, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

The conjugates can also include targeting groups, e.g., a cell or tissue targeting agent or group, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent fucose, or aptamers.

The targeting group can be any ligand that is capable of targeting a specific receptor. Examples include, without limitation, folate, GalNAc, galactose, mannose, mannose-6P, aptamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

In still other embodiments, glycan-interacting antibodies are covalently conjugated to a cell penetrating polypeptide. The cell-penetrating peptide may also include a signal sequence. The conjugates of the invention can be designed to have increased stability; increased cell transfection; and/or altered biodistribution (e.g., targeted to specific tissues or cell types).

Conjugating moieties may be added to glycan-interacting antibodies such that they allow labeling or flagging targets for clearance. Such tagging/flagging molecules include, but are not limited to ubiquitin, fluorescent molecules, human influenza hemaglutinin (HA), c-myc [a 10 amino acid segment of the human protooncogene myc with sequence EQKLISEEDL (SEQ ID NO: 5)], histidine (His), flag [a short peptide of sequence DYKDDDDK (SEQ ID NO: 6)], glutathione S-transferase (GST), V5 (a paramyxovirus of simian virus 5 epitope), biotin, avidin, streptavidin, horse radish peroxidase (HRP) and digoxigenin.

In some embodiments, glycan-interacting antibodies may be combined with one another or other molecule in the treatment of a disease or condition. In some cases, glycan-interacting antibodies may be used in combination with one or more immune checkpoint inhibitors, including those described herein (e.g., a PD-1 or CTLA4 inhibitor).

### Nucleic Acids

The present invention embraces nucleic acid molecules. In some embodiments, nucleic acids encode antibodies of the invention (including, but not limited to antibodies, antibody fragments, intrabodies and chimeric receptor antigens). Such nucleic acid molecules include, without limitation, DNA molecules, RNA molecules, polynucleotides, oligonucleotides, mRNA molecules, vectors, plasmids and other constructs. As used herein, the term “construct” refers to any recombinant nucleic acid molecule including, but not limited to plasmids, cosmids, autonomously replicating polynucleotide molecules or linear or circular single-stranded or double-stranded DNA or RNA polynucleotide molecules. The present invention also embraces cells programmed or generated to express nucleic acid molecules encoding glycan-interacting antibodies. Such cells may be generated through the use of transfection, electroporation, viral delivery and the like. Viruses engineered with constructs of the invention may include, but are not limited to lentiviruses, adenoviruses, adeno-associated viruses and phages. In some cases, nucleic acids of the invention include codon-optimized nucleic acids. Methods of generating codon-optimized nucleic acids are known in the art and may include, but are not limited to those described in U.S. Pat. Nos. 5,786,464 and 6,114,148, the contents of each of which are herein incorporated by reference in their entirety. In some embodiments, nucleic acid sequences are codon optimized to improve protein expression or to remove cryptic splice sites.

### II. Methods and Uses

### Therapeutics

Methods of the present disclosure include methods of targeting one or more cells with anti-glycan antibodies. Such cells may be cancer cells. In some cases, the cells are tumor cells. In some embodiments, methods of the present disclosure are used to prevent, reduce, inhibit, or eliminate one or more cancer cells or tumors. Tumors may include MDSCs. In some cases, such tumors may include a population of cells expressing one or more markers of MDSCs. Such markers may include one or more of CD45, CD133, and STn. Some populations of cells expressing one or more markers of MDSCs make up from about 1% to about 100% of a tumor. For example, tumors may include at least 1%, at least 5%, at least 10%, at least 20%, at least 30%, at least 40%, at least 50%, at least 60%, at least 70%, at least 80%, at least 90%, or 100% cells with one or more markers of MDSCs.

In some embodiments, the present disclosure provides methods of targeting MDSCs. Such methods may include contacting a subject or sample with an anti-glycan antibody. According to such methods, the subject or sample may include at least one MDSC. The anti-glycan antibody may be an anti-STn antibody. Some such anti-STn antibodies may include any of those described herein or variants of any of those antibodies described herein (e.g., antibody fragments, optimized variants, humanized variants, chimeric variants, etc.).

In some embodiments, methods of the present disclosure include methods of increasing antitumor cell activity. Such methods may include contacting a subject or sample with an anti-glycan antibody. According to such methods, the subject or sample may include at least one MDSC. The anti-glycan antibody may be an anti-STn antibody. Some such anti-STn antibodies may include any of those described herein or variants of any of those antibodies described herein (e.g., antibody fragments, optimized variants, humanized variants, chimeric variants, etc.).

Methods of the present disclosure include methods of treating a subject, wherein the subject has at least one tumor. Such methods may include providing an anti-STn antibody to the subject. Anti-STn antibodies may include any of those described herein or variants of any of those antibodies described herein (e.g., antibody fragments, optimized variants, humanized variants, chimeric variants, etc.). Tumors may include any tumor type. Such tumor types may include, but are not limited to, tumors from the breast, colon, lung, bladder, cervix, ovary, endometrium, gastric system, kidney, stomach, prostate, pancreas, and liver. In some cases, tumors may be metastatic tumors.

Antibodies used according to any of the methods described herein may be IgG antibodies. In some cases, the antibodies are antibody-drug conjugates. Such antibodies may be conjugated to a cytotoxic agent. Cytotoxic agents may include, but are not limited to cytoskeletal inhibitors [e.g., tubulin polymerization inhibitors such as maytansines or auristatins (e.g. monomethyl auristatin E [MMAE] and monomethyl auristatin F [MMAF]), and kinesin spindle protein (KSP) inhibitors], DNA damaging agents (e.g., calicheamicins, duocarmycins, and pyrrolobenzodiazepine dimers such as talirine and tesirine), topoisomerase inhibitors [e.g., camptothecin compounds or derivatives such as 7-ethyl-10-hydroxycamptothecin (SN-38) and exatecan derivative DXd], transcription inhibitors (e.g., RNA polymerase inhibitors such as amanitin), and kinase inhibitors [e.g., phosphoinositide 3-kinase (PI3K) inhibitors or mitogen-activated protein kinase kinase (MEK) inhibitors].

In some embodiments, methods of the present disclosure include methods of identifying a subject for treatment with an anti-glycan antibody. Such methods may include contacting a subject or sample with an anti-glycan antibody. According to such methods, the subject or sample may include at least one MDSC. The anti-glycan antibody may be an anti-STn antibody. Some such anti-STn antibodies may include any of those described herein or variants of any of those antibodies described herein (e.g., antibody fragments, optimized variants, humanized variants, chimeric variants, etc.).

In some embodiments, the present disclosure provides a method of reducing MDSC levels in subject tissues and/or fluids by administering an anti-STn antibody (e.g., SIA01). The levels may be granulocytic and/or monocytic MDSC levels, wherein the granulocytic and/or monocytic MDSCs express STn. In some embodiments, subject tissues include tumor and/or spleen tissue. MDSC STn expression may be linked to tumor STn expression. In some embodiments, the percentage of tumor cells expressing STn may be reduced by anti-STn administration, resulting in decreased levels of MDSCs expressing STn. The tumor may include, but is not limited to, one or more of a bladder tumor, a colon tumor, an endometrial tumor, a gastric tumor, a kidney tumor, an ovarian tumor, and a pancreatic tumor.

Anti-STn antibodies used to reduce MDSC levels may be antibody-drug conjugates. The antibody-drug conjugate may include a cytotoxic agent. In some embodiments, the antibody-drug conjugate includes one or more of an auristatin, a maytansine, a tubulysin, a vinca alkaloid, a pyrrolobenzodiazepine dimer, a camptothecin, a duocarmycin, an amanitin, a PI3K inhibitor, a nucleotide analog, and a MEK inhibitor.

### Cancer-Related Applications

Aberrant glycosylation is a hallmark of cancer cell transformation. Multiple aberrant glycosylation forms have been described in human cancers, identifying specific tumor-associated carbohydrate antigens (TACAs) as a class of cell surface molecules suitable for specific tumor targeting (Cheever, M. A. et al., Clin Cancer Res. 2009 Sep. 1; 15(17):5323-37). TACA antigen expression has been found in epithelial cancers including, but not limited to, breast, colon, lung, bladder, cervical, ovarian, stomach, prostate, and liver. TACA antigen expression has been found in embryonal cancers including, but not limited to, yolk sac tumors and seminomas. In addition, TACA antigen expression has been found in many melanomas, carcinomas, and leukemias of various tissues (Heimburg-Molinaro et al., Vaccine. 2011 Nov. 8: 29(48):8802-8826). Antibodies of the present invention that target one or more TACA are referred to herein as “anti-TACA antibodies.”

MUC1 is a key cell surface glycoprotein that is normally extensively glycosylated but is under glycosylated in tumor cells. Sparse glycosylation of MUC1 leads to exposure of immunogenic antigens. These may be along the MUC1 core peptide sequence or along core carbohydrate residues. These TACAs include, but are not limited to N-acetylgalactosamine (Tn), sialyl(α2,6)N-acetylgalactosamine (STn) and galactose(β1-3)N-acetylgalactosamine (also known as Thomsen-Friedenreich antigen or TF). It has been estimated that about 80% of all carcinomas express Tn among the core carbohydrates of MUC1 with STn being strongly expressed on human carcinoma cells and linked to cancer progression and metastasis. With few exceptions, Tn and STn are not expressed in normal healthy tissues. Sialic acid forms a prominent epitope on STn. The invention takes advantage of the fact that aberrant Neu5Gc-STn (GcSTn) glycan expression appears to be highly specific to various carcinomas.

In the case of MUC1, Neu5Gc incorporation into STn yields a tumor-specific target, a site that is an attractive target for antibody-based therapies to treat tumor tissue. In some embodiments of the present invention, glycan-interacting antibodies target MUC1 expressing cancer cells that include Neu5Gc. To date, Neu5Gc has been detected in glycoconjugates from a number of human cancer tissues including, but not limited to colon cancer, retinoblastoma tissue, melanoma, breast cancer and yolk sac tumor tissue. In some embodiments of the present invention, methods are contemplated for glycan-interacting antibody treatment of these forms of cancer as well as other forms of cancer, not specifically listed here, characterized by the presence of cancer cells that include Neu5Gc.

Additional antigens that include glycans have been identified that are expressed in correlation with cancer (Heimburg-Molinaro, J. et al., Cancer vaccines and carbohydrate epitopes. Vaccine. 2011 Nov. 8; 29(48):8802-26). These tumor-associated carbohydrate antigens include, but are not limited to blood group Lewis related antigens [including, but not limited to Lewis^{Y} (Le^{Y}), Lewis^{X} (Le^{X}), Sialyl Lewis^{X} (SLe^{X}) and Sialyl Lewis^{A} (SLe^{A})], glycosphingolipid-related antigens [including, but not limited to Globo H, stage-specific embryonic antigen-3 (SSEA-3) and glycosphingolipids that include sialic acid], ganglioside-related antigens [including, but not limited to gangliosides GD2, GD3, GM2, fucosyl GM1 and Neu5GcGM3] and polysialic acid-related antigens.

In some embodiments, therapeutics of the present invention may be directed toward Lewis blood group antigens. Lewis blood group antigens include a fucose residue linked to GlcNAc by an al-3 linkage or an al-4 linkage. They may be found on both glycolipids and glycoproteins. Lewis blood group antigens may be found in the body fluid of individuals that are secretors of these antigens. Their appearance on red cells is due to absorption of Lewis antigens from the serum by the red cells.

In some embodiments, therapeutics of the present invention may be directed toward Le^{Y}. Le^{Y} (also known as CD174) is made up of Galβ1,4GlcNAC and includes α1,2- as well as α1,3-linked fucose residues yielding the Fucα(1,2)Galβ(1,4)Fucα(1,3)GlcNAc epitope. It is synthesized from the H antigen by α1,3 fucosyltransferases which attach the α1,3 fucose to the GlcNAc residue of the parent chain. Le^{Y} may be expressed in a variety of cancers including, but not limited to ovarian, breast, prostate, colon, lung and epithelial. Due to its low expression level in normal tissues and elevated expression level in many cancers, the Le^{Y} antigen is an attractive target for therapeutic antibodies.

In some embodiments, therapeutics of the present invention may be directed toward Le^{X}. Le^{X} includes the epitope Galβ1-4(Fucα1-3)GlcNAcβ-R. It is also known as CD15 and stage-specific embryonic antigen-1 (SSEA-1). This antigen was first recognized as being immunoreactive with sera taken from a mouse subjected to immunization with F9 teratocarcinoma cells. Le^{X} was also found to correlate with embryonic development at specific stages. It is also expressed in a variety of tissues both in the presence and absence of cancer, but can also be found in breast and ovarian cancers where it is only expressed by cancerous cells.

In some embodiments, therapeutics of the present invention may be directed toward SLe^{A} and/or SLe^{X}. SLe^{A} and SLe^{X} include the structures Neu5Acα2-3Galβ1-3(Fucα1-4)GlcNAcβ-R and Neu5Acα2-3Galβol-4(Fucα1-3)GlcNAcβ-R respectively. Their expression is upregulated in cancer cells. The presence of these antigens in serum correlates with malignancy and poor prognosis. SLe^{X} is mostly found as a mucin terminal epitope. It is expressed in a number of different cancers including breast, ovarian, melanoma, colon, liver, lung and prostate. In some embodiments of the present invention, SLe^{A} and SLe^{X} targets include Neu5Gc (referred to herein as GcSLe^{A} and GcSLe^{X}, respectively).

In some embodiments, therapeutics of the present invention may be directed toward glycolipids and/or epitopes present on glycolipids, including, but not limited to glycosphingolipids. Glycosphingolipids include the lipid ceramide linked to a glycan by the ceramide hydroxyl group. On the cell membrane, glycosphingolipids form clusters referred to as “lipid rafts”.

In some embodiments, therapeutics of the present invention may be directed toward Globo H. Globo H is a cancer-related glycosphingolipid first identified in breast cancer cells. The glycan portion of Globo H includes Fucα(1-2)Galβ(1-3)GalNAcβ(1-3)Galα(1-4)Galβ(1-4)Glcβ(1). Although found in a number of normal epithelial tissues, Globo H has been identified in association with many tumor tissues including, but not limited to, small cell lung, breast, prostate, lung, pancreatic, gastric, ovarian and endometrial tumors.

In some embodiments, therapeutics of the present invention may be directed toward gangliosides. Gangliosides are glycosphingolipids that include one or more sialic acid. According to ganglioside nomenclature, G is used as an abbreviation for ganglioside. This abbreviation is followed by the letters M, D, or T referring to the number of sialic acid residues attached (1, 2 or 3 respectively). Finally, the numbers 1, 2 or 3 are used to refer to the order of the distance each migrates when analyzed by thin layer chromatography (wherein 3 travels the greatest distance, followed by 2, and then 1). Gangliosides are known to be involved in cancer-related growth and metastasis and may be expressed on the cell surface of tumor cells. Gangliosides expressed on tumor cells may include, but are not limited to GD2, GD3, GM2 and fucosyl GM1 (also referred to herein as Fuc-GM1). In some embodiments of the present invention, glycan-interacting antibodies are directed toward GD3. GD3 is a regulator of cell growth. In some embodiments, GD3-directed antibodies are used to modulate cell growth and/or angiogenesis. In some embodiments, GD3-directed antibodies are used to modulate cell attachment. In some embodiments of the present invention, glycan interacting antibodies are directed toward GM2. In some embodiments, GM2-directed antibodies are used to modulate cell to cell contact. In some embodiments, ganglioside targets of the present invention include one or more Neu5Gc residue. In some embodiments, such targets may include a GM3 variant having Neu5Gc (referred to herein as GcGM3). The glycan component of GcGM3 is Neu5Gcα2-3Galβ1-4Glc. GcGM3 is a known component of tumor cells.

In some embodiments, TACAs targeted by anti-TACA antibodies of the present invention may include, but are not limited to any of those listed in US Publication Nos. US2013/0236486A1, US2013/0108624A1, US2010/0178292A1, US2010/0104572A1, US2012/0039984A1, US2009/0196916A1, and US2009/0041836A1, the contents of each of which are herein incorporated by reference in their entirety.

In some embodiments, methods of the present disclosure include targeting one or more MDSCs, wherein the MDSCs include at least one TACA. Such methods may include the use of an anti-glycan antibody.

### STn in Cancer

The immune system has multiple mechanisms for promoting anti-tumor cell immune activity including both innate and adaptive immune activity. As used herein, the term “anti-tumor cell immune activity” refers to any activity of the immune system that kills or prevents growth and/or proliferation of tumor cells. In some cases, anti-tumor immune activity includes recognition and tumor cell killing by natural killer (NK) cells and phagocytosis by macrophages. Adaptive anti-tumor immune responses include tumor antigen uptake and presentation by antigen presenting cells (APCs,) such as dendritic cells (DCs,) leading to modulation of T cell anti-tumor activity and/or expansion of B cells with secretion of tumor-specific antibodies. The binding of tumor-specific antibodies to tumors can lead to antibody-dependent cellular cytotoxicity (ADCC) and complement-dependent cytotoxicity (CDC) mechanisms of tumor cell death.

As used herein, the term “immune-resistant tumor cell” refers to a tumor cell that reduces or evades anti-tumor cell immune activity. Some studies indicate that the expression of STn (a known TACA) on tumor cell surfaces or secreted into the tumor cell microenvironment can promote tumor cell evasion of anti-tumor immune activity. As used herein, the term “tumor cell microenvironment” refers to any area adjacent to or surrounding a tumor cell. Such areas include, but are not limited to areas between tumor cells, between tumor and non-tumor cells, surrounding fluids and surrounding components of the extracellular matrix.

Sialylated mucins having STn were demonstrated by Ogata et al to reduce NK cell targeting of tumor cells (Ogata, S. et al., 1992. Canc. Res. 52:4741-6, the contents of which are herein incorporated by reference in their entirety). This study found that the presence of ovine, bovine and porcine submaxillary mucin (OSM, BSM and PSM, respectively) led to nearly one hundred percent inhibition of cytotoxicity (see Table 2 of Ogata et al). Further studies by Jandus et al, demonstrate that some tumor cells can evade NK destruction due to the expression of sialoglycan ligands that can interact with NK cell siglec receptors, leading to NK inhibition (Jandus, C. et al., 2014, JCI. pii: 65899, the contents of which are herein incorporated by reference in their entirety).

Studies by Toda et al., demonstrate that STn may bind CD22 receptors on B cells, leading to decreased signal transduction and reduced B cell activation (Toda, M. et al., 2008. Biochem Biophys Res Commun. 372(1):45-50, the contents of which are herein incorporated by reference in their entirety). Dendritic cells (DCs) can affect adaptive immune activity by modulating T cell activity. Studies by Carrascal et al found that STn expression by bladder cancer cells induced tolerance in DCs, reducing their ability to induce anti-tumor cell immune activity in T cells (Carrascal, M A et al., 2014. Mol Oncol. pii: S1574-7891(14)00047-7, the contents of which are herein incorporated by reference in their entirety). These studies revealed that DCs coming into contact with STn-positive bladder cancer cells displayed a tolerogenic expression profile with low expression of CD80, CD86, IL-12 and TNF-α. Further, DCs were found to modulate regulatory T cells such that the T cells had low expression of IFNγ and high expression of FoxP3. Other studies by van Vliet and others, indicate that DC surface expression of macrophage galactose-type lectin (MGL) can lead to targeting of those cells to tumor tissues (van Vliet, S J., 2007. Amsterdam: Vrije Universiteit. p 1-232 and van Vliet, S J. et al., 2008. J Immunol. 181(5):3148-55, Nollau, P. et al., 2013. J Histochem Cytochem. 61(3):199-205, the contents of each of which are herein incorporated by reference in their entirety). DCs arriving at tissues due to MGL interactions may influence T helper (Th) cells in one of three ways. DCs can induce T cell tolerance, T cell immune activity or downregulation of effector T cells. MGL has been shown to bind to both AcSTn and GcSTn and the affinity has been analyzed in depth (Mortezai, N. et al., 2013. Glycobiology. 23(7):844-52, the contents of which are herein incorporated by reference in their entirety). Interestingly, MUC1 expression on tumors has been shown to lead to T cell tolerance, protecting tumor cells from immune eradication.

In some embodiments, glycan-interacting antibodies (including, but not limited to anti-STn antibodies) of the present invention may be used to treat subjects having one or more tumor cells expressing one or more TACAs. In some cases, glycan-interacting antibodies (including, but not limited to anti-STn antibodies) of the invention may be used to increase anti-tumor cell immune activity toward tumor cells expressing STn. Such antibodies may increase the adaptive immune response and/or the innate immune response toward immune-resistant tumor cells. Some glycan-interacting antibodies may be used to increase NK anti-tumor cell activity. Such glycan-interacting antibodies may, in some cases, block the interaction between glycan receptors expressed on NK cells and STn glycans on cancer cells or in surrounding tissues.

In some embodiments, glycan-interacting antibodies (including, but not limited to anti-STn antibodies) of the invention may be used to increase B cell anti-tumor cell activity. Such antibodies may reduce the interaction between CD22 receptors on B cells and STn glycans on cancer cells or in surrounding tissues. A study by Sjoberg et al. demonstrates that 9-O-acetylation of α2,6-linked sialic acids on glycoproteins also reduced interaction between B cell CD22 receptors and such glycoproteins (Sjoberg, E. R. et al. 1994. JCB. 126(2): 549-562). Another study by Shi et al. reveals that higher levels of 9-O-acetylated sialic acid residues on murine erythroleukemia cells makes these cells more susceptible to complement-mediated lysis (Shi, W-X. et al., 1996. J of Biol Chem. 271(49): 31526-32, the contents of which are herein incorporated by reference in their entirety). In some embodiments, anti-STn antibodies of the invention are capable of selectively binding non-9-O-acetylated STn, reducing overall STn binding, but reducing tumor cell growth and/or proliferation. (e.g. through increased B cell anti-tumor activity and increased complement-mediated tumor cell destruction). In some embodiments, glycan-interacting antibodies (including, but not limited to anti-STn antibodies) of the invention may be used to increase DC anti-tumor activity. Such antibodies may be used to reduce DC tolerance to tumor cells. Reduced DC tolerance may include increasing DC expression of CD80, CD86, IL-12 and/or TNF-α. In some cases, DC anti-tumor cell activity may include promotion of T cell anti-tumor cell activity. Such antibodies may prevent binding between DC MGL and glycans expressed on or around cancer cells.

A study by Ibrahim et al. suggests that high levels of anti-STn antibodies along with endocrine therapy may increase overall survival and time to progression (TTP) in women with metastatic breast cancer (Ibrahim, N. K. et al., 2013. 4(7): 577-584, the contents of which are herein incorporated by reference in their entirety). In this study, anti-STn antibody levels were elevated after vaccination with STn linked to keyhole-limpet Hemocyanin (KLH). In some embodiments, anti-STn antibodies of the invention may be used in combination with endocrine therapy (e.g. tamoxifen and/or an aromatase inhibitor).

In some embodiments, glycan-interacting antibodies of the invention may be used to reduce or eliminate cancerous cells and/or cells expressing STn. Such cells include cells that may be part of a tumor.

In some cases, the present invention provides methods of reducing tumor volumes by administering anti-glycan antibodies of the invention to subjects with one or more tumors. Reduction in tumor volumes may be determined by comparing tumor volumes in a subject before and after treatment, or by comparing tumor volumes between anti-glycan antibody-treated and control treated subjects.

In some cases, anti-glycan antibodies of the invention may be administered to achieve a desired percent reduction in tumor volume in a subject. This may be assessed by determining the volume of one or more tumors (e.g., through the use of calipers or imaging techniques like CT scan) in a subject before and after treatment with an anti-glycan antibody and then calculating the percent reduction in tumor volume from the two values. In some embodiments, tumor volume in subjects treated with anti-glycan antibodies may be reduced by from about 0.10% to about 2%, from about 1% to about 5%, from about 3% to about 12%, from about 10% to about 30%, from about 20% to about 50%, from about 40% to about 60%, from about 50% to about 75%, from about 60% to about 85%, or from about 80% to about 99%. In some cases, tumor volume in subjects treated with anti-glycan antibodies may be reduced by at least 1%, by at least 5%, by at least 10%, by at least 20%, by at least 40%, by at least 50%, by at least 60%, by at least 80%, by at least 85%, by at least 90%, by at least 95%, by at least 98%, by at least 99%, or by 100%.

In some cases, anti-glycan antibodies of the invention may be administered to achieve a desired percent tumor growth inhibition (% T/C). % T/C is calculated by determining tumor volumes in treated subjects and comparing them to tumor volumes in non-treated or placebo-treated subjects. In some embodiments, the present invention provides methods of reducing tumor volume in a subject by administering an anti-glycan antibody, wherein the % T/C is from about 0.10% to about 1%, from about 0.5% to about 5%, from about 2% to about 20%, from about 3% to about 16%, from about 10% to about 30%, from about 20% to about 60%, or from about 40% to about 80%. In some cases, the % T/C is at least 80%. In some cases, the % T/C is less than 0.1%.

In some embodiments, antibodies used to reduce tumor volumes in subjects may be selected based on their ability to bind cell surface glycans (e.g., STn) and/or their ability to kill cancerous cells. In some instances, antibodies may be selected based on their half-maximal effective concentration (EC₅₀) for binding cells having cell surface STn. EC₅₀ values for such antibodies may be determined, e.g., through flow cytometry analysis with cells having cell surface STn. Such antibodies may have EC50 values of from about 0.1 nM to about 2 nM, from about 0.5 nM to about 5 nM, from about 1 nM to about 10 nM, from about 5 nM to about 20 nM, or from about 10 nM to about 30 nM.

In some embodiments, the present invention provides methods of killing cancer cells, such as tumor cells, by administering one or more antibodies presented herein.

In some embodiments, the present disclosure provides a method of identifying a subject in need of anti-STn antibody treatment by isolating cancer cells (including, but not limited to cancer stem cells) and/or obtaining biopsy material from a subject and screening the cancer cells and/or biopsy material for STn expression. According to such methods, subjects with cancer cells and/or biopsy material expressing STn are deemed likely to benefit from anti-STn antibody treatment or to be in need of anti-STn antibody treatment (e.g., treatment with one or more antibody described herein). In some cases, antibodies described herein may be used for screening of cancer cells and/or biopsy material. Cancer cells may be screened in vitro by culturing the cancer cells and detecting STn expression using standard immunological assays (e.g., ELISA, Western blot, or other standard immunological assays). In some cases, cancer cells may be screened for STn expression using flow cytometry techniques. In other embodiments, cancer cells may be grown in culture and tested for viability after treatment with anti-STn antibodies that are antibody-drug conjugates (ADCs). Such ADCs may include a cytotoxic agent, including, but not limited to those described herein. Cytotoxic agents may include MMAE. Anti-STn antibodies may include humanized antibodies, including, but not limited to, those described herein. In other embodiments, cancer cells may be screened by using the cancer cells to form tumors in mice (e.g., NOD/SCID mice). The tumors developed in mice may be screened by preparing tissue sections from such tumors and subjecting the tissue sections to immunohistochemical analysis using anti-STn antibodies, including, but not limited to anti-STn antibodies described herein. In some cases, the tumors formed in mice may be assessed for changes in tumor volume after treatment of the mice with anti-STn antibodies, including, but not limited to anti-STn antibodies described herein. Such anti-STn antibodies may include ADCs. ADCs may include one or more cytotoxic agent, including, but not limited to any of those described herein (e.g., MMAE). Subjects with cancer cells that demonstrate STn expression after screening may be determined to be in need of anti-STn antibody treatment.

In some embodiments, the present disclosure provides a method of identifying an antibody suitable for treating cancer by isolating cancer cells (including, but not limited to cancer stem cells) from a subject, screening the cancer cells for STn expression, and contacting STn-expressing cancer cells with one or more candidate antibodies specific for STn to determine whether any of the one or more candidate antibodies are able to bind the cancer cells. As used herein, the term “candidate antibody” refers to an antibody or one of a group of antibodies that are being evaluated for one or more purposes. Subject cancer cells may be screened in vitro by culturing the cancer cells and detecting STn expression using STn-detecting antibodies with standard immunological assays (e.g., ELISA, Western blot, or other standard immunological assays) or using flow cytometry techniques. As used herein, the term “STn-detecting antibody” refers to an antibody that binds STn and that allows for observation of such binding either through the presence of an incorporated detectable label or through the use of a secondary antibody having a detectable label. In other embodiments, screening the cancer cells may involve using them to form tumors in mice (e.g., NOD/SCID mice). Screening may be carried out by assessing the mouse tumors for expression of STn or for reduction in volume after administration of anti-STn antibodies, including, but not limited to ADCs.

In some embodiments, the present invention includes methods of evaluating the suitability of an antibody for treating cancer in a subject by obtaining cancer cells from a subject, using the cancer cells to form tumors in mice (e.g., NOD/SCID mice), administering an anti-STn antibody to the mice, and measuring changes in tumor volume in the mice, wherein if the tumor volume in the mice is decreased, the anti-STn antibody is determined to be suitable for treating cancer in the subject. In some cases, the anti-STn antibodies are administered multiple times. According to such methods, antibodies may be administered hourly, daily, weekly, monthly, and/or yearly. In some cases, antibodies are administered weekly for a period of from about 2 to about 12 weeks. In some cases, antibodies are administered weekly for a period of at least 12 weeks.

STn expression has been implicated in contributing to the metastatic potential of cancer cells. According to some methods of the disclosure, glycan-interacting antibodies may be used to reduce metastasis. Such methods may include the reduction of metastasis by from about 1% to about 15%, from about 5% to about 25%, from about 10% to about 50%, from about 20% to about 60%, from about 30% to about 70%, from about 40% to about 80%, from about 50% to about 90%, from about 75% to about 95%, or at least 95%.

### Cancer Stem Cells as Therapy Targets

Cancer stem cells or CSCs (also called tumor initiating cells) are a subset of cancer cells within a heterogeneous tumor population that drive the initiation, growth, dissemination, and recurrence of primary and metastatic tumors (Karsten and Goletz, SpringerPlus, 2013, 2, 301), which can occur in varying proportions of the total population depending on tumor type. CSCs are distinguished from terminally differentiated cells by their capacity to self-renew and give rise to non-CSC, differentiated progeny (Gupta et al., Nature medicine, 2009, 15, 1010-1012). These properties are akin to those of normal stem cells. Such distinctions between normal stem cells and CSCs have important implications for therapy.

An increasing number of cell-surface biomarkers have been identified that purport to differentiate CSCs from their non-CSC counterparts (Medema et al., Nature cell biology, 2013, 15, 338-344; Zoller, Cancer, 2011, 11, 254-267). These may include, but are not limited to CD44, CD133, CD117, and aldehyde dehydrogenase isoform 1 (ALDH1). Although some of these derive from studies of mouse tumors and human cell lines, others have been validated using primary human tumor samples. One of these, the membrane-spanning CD44 glycoprotein, or hyaluronan receptor, which is a well-known constituent of a variety of tumor types, has also more recently found acceptance as a bona fide CSC marker in human cancers, and in fact is the one most frequently observed (Lobo et al., 2007, 23, 675-699).

CD44 exists in several variant isoforms generated by alternative splicing events occurring among the 20 exons and 19 introns of the full-length CD44 gene (Williams et al, Experimental biology and medicine, 2013, 238, 324-338). Growing experimental evidence points to the supporting role of CD44 and its variants in contributing to the innate metastatic and drug resistant phenotype of CSCs (Negi et al., Journal of drug targeting, 2012, 20, 561-573), in part due to modulation of intracellular signal transduction pathways (Williams et al, Experimental biology and medicine, 2013, 238, 324-338). Additionally, patients with triple negative breast cancer, along with several other cancer types, that display high levels of CD44 cells are known to have a poor prognosis and higher mortality (Negi et al., Journal of drug targeting, 2012, 20, 561-573). These observations support the notion that targeting CD44 offers a means of treating cancer through inhibition or elimination of CSCs, in addition to mature cancer cells. Indeed, numerous approaches to targeting CD44 have been attempted experimentally with varying degrees of success. These include a wide range of technologies that include the use of conjugated and unconjugated antibodies, nano-carrier drug systems, and hyaluronan-conjugated drugs (Negi et al., Journal of drug targeting, 2012, 20, 561-573). In several instances, however, toxic effects were observed in in vivo studies; these untoward side effects may be attributable to the widespread occurrence of CD44 and variants on the membranes of most vertebrate cells (Naor et al., Seminars in cancer biology, 2008, 18, 260-267), in addition to its presence on the surface of the targeted CSCs and mature tumor cells. Targeting CD44 protein, which is a constituent of normal human stem cells (Williams et al, Experimental biology and medicine, 2013, 238, 324-338), can also harm normal stem cell function (Leth-Larsen et al., Molecular medicine, 2012, 18, 1109-1121). Although a large body of research points to the desirability of targeting CD44 protein on CSCs, as well as on mature tumor cells, the intrinsic problem with this approach remains the present difficulty in designing inhibitors that will spare normal tissue as well as normal stem cells.

Another well-known tumor antigen with implications to CSC biology is the epithelial mucin MUC1, a membrane tethered glycoprotein that is differentially expressed at high levels on the majority of adenocarcinomas but at low levels or not at all on normal epithelial cells. MUC1 has recently been identified as a CSC biomarker on a variety of neoplasias including breast (Engelmann et al., Cancer research, 2008, 68, 2419-2426), and pancreatic cancers, where its expression is correlated with high metastasis and poor prognosis. As a constituent of CSCs, MUC1 has been shown to function in cell adhesion, proliferation, survival, and signaling (Engelmann et al., Cancer research, 2008, 68, 2419-2426) and may also be co-expressed with CD44 (Leth-Larsen et al., Molecular medicine, 2012, 18, 1109-1121). Immunotherapeutic approaches for targeting MUC1 in cancer are being pursued using vaccines as well as other approaches, but primarily in the context of mature cancer cell therapy (Julien et al., Biomolecules, 2012, 2, 435-466; Acres et al., Expert review of vaccines, 2005, 4, 493-502).

Cancer stem cells have been hypothesized to be generated through the epithelial-to-mesenchymal (EMT) transition (Gupta et al., Nature medicine, 2009, 15, 1010-1012), and/or reversely the mesenchymal-to-epithelial (MET) transition that occurs at the site of metastasis (Leth-Larsen et al., Molecular medicine, 2012, 18, 1109-1121) (also called CSCs plasticity where non-CSCs can give rise to CSCs). This discovery further underscores the need to eliminate both CSCs and non-CSCs in a tumor population.

Recent studies with enriched CSC populations has revealed that these cells, unlike the bulk of the tumor, are relatively quiescent and are preferentially resistant to many types of current therapies, including chemotherapy and radiation (Leth-Larsen et al., Molecular medicine, 2012, 18, 1109-1121). Thus, current therapeutic strategies target non-CSC components of the tumor, leaving CSCs largely unaffected only to re-emerge after appropriate cues to reform recurrent primary tumors at the initial site or to disseminate to distant sites, colonize, and create metastatic disease, the major cause of cancer mortality.

Current understanding of the properties of CSCs clearly emphasized the need not only to target the bulk of cells present in tumors, as is current practice, but also the CSC compartment in order to potentially effect complete cures.

As discussed above, strategies that have been developed based on tumor (including CSCs) associated biomarkers face a challenge that most cancer biomarkers are also present in normal cells including normal stem cells. A therapy that targets a protein biomarker to eliminate CSCs, may also target normal stem cells, causing elimination of normal cells.

### Tumor-Specific Glycans in CSCs

Aberrant forms of glycosylation, including appearance of the Thomsen-nouveau (Tn) antigen (GalNAc—O-Ser/Thr), have been described in numerous human cancers, identifying glycans as an entirely novel class of tumor-associated carbohydrate antigens suitable for specific tumor targeting (Rabu et al., Future oncology, 2012, 8, 943-960). The formation of the sialyl derivative of Tn (STn) is mediated by the sialyl transferase ST6GalNAc-I which adds sialic acid in an α2,6 linkage to the Tn antigen. The sialylation of STn prevents further sugar additions, thus truncating further glycan extensions (Schultz et al., Cancer metastasis reviews, 2012, 31, 501-518).

While the presence of STn in normal adult human tissues is rare, STn occurs in various human cancers, including ovarian, bladder, breast, cervical, colon, and lung cancer, among others (Ferreira et al., Molecular oncology, 2013, 7, 719-731; Kinney et al., Cancer, 1997, 80, 2240-2249). Further, the presence of STn in tumors is associated with metastatic disease, poor prognosis, and reduced overall survival (Ferreira et al., Molecular oncology, 2013, 7, 719-731; Kinney et al., Cancer, 1997, 80, 2240-2249); therefore, STn is considered a highly attractive target for cancer detection and therapy. There are two distinct forms of sialic acid—Neu5Ac and Neu5Gc—located at the terminal position of STn. The Neu5Ac-sialylated form is predominant in humans since humans cannot synthesize Neu5Gc due to an inactive CMP-Neu5Ac hydroxylase (CMAH) gene. However, consumption of Neu5Gc-rich foods leads to foreign Neu5Gc incorporation into human cells, especially in carcinomas. Previous studies have shown that solid tumors take up and express the Neu5Gc form of sialic acid (Inoue et al., Glycobiology, 2010, 20, 752-762; Malykh et al., Biochimie, 2001, 83, 623-634; Padler-Karavani et al., Cancer research, 2011, 71, 3352-3363). mAbs that bind to both glyco-isoforms of STn that are potential cancer targets: Neu5Ac-STn (AcSTn) and Neu5Gc-STn (GcSTn) (i.e., designated as pan-STn antibodies).

STn accumulation is associated with specific somatic mutations observed repeatedly in solid tumors and with the inactivation of the gene that encodes the molecular chaperone Core 1 Beta3-Galactosyltransferase-Specific Molecular Chaperone (COSMC), which is required for the formation of active T-synthase (Ju et al., Nature, 2005, 437, 125). T-synthase competes with ST6GalNAc-I for the GalNAc substrate and therefore when inactivated by mutation results in elevated STn synthesis. Additionally, STn accumulation can also result from increased expression of ST6GalNAc-I, which is often observed (Brockhausen et al., Biological chemistry, 2001, 382, 219-232; Ikehara et al., Glycobiology, 1999, 9, 1213-1224). De novo expression of STn can modulate carcinoma cells, change the malignant phenotype, and lead to more aggressive cell behaviors (Pinho et al., Cancer letters, 2007, 249, 157-170). As such, STn is not only an interesting cancer biomarker and therapeutic target, but interfering with STn function offers the intriguing potential to have significant functional, anti-metastatic therapeutic benefits.

Although it is well-known that glycosylation of cellular glycoproteins is altered in cancer, it appears that aberrant glycosylation is selective with respect to both the glycoprotein and glycan in question. In fact, in human tumor CSCs only CD44 and MUC1 are major carriers of the STn antigen (Cazet et al., Breast cancer research: BCR, 2010, 12,204; Julien et al., Glycobiology, 2006, 16, 54-64), immediately suggesting a selective approach for targeting not only mature tumor cells but also CSCs. Whereas MUC1 is a normal surface constituent of some epithelial cells where it serves a barrier function, tumor-associated MUC1 is characterized by hypoglycosylation and increased sialylation on CSCs in the same fashion as observed in mature cancer cells, with STn appearing as a specific marker for both CSCs and mature tumor cells (Curry et al., Journal of surgical oncology, 2013, 107, 713-722). The aberrant oligosaccharide profile of MUC1 gives rise to the expression of neomarkers such as sialyl-Le^{a} (used in the CA19-9 test), sialyl-Le^{x}, and sialyl-Tn (TAG-72), as well as the cryptic epitopes such as Tn in cancer cells (e.g., CSCs). In addition, because of underglycosylation, the peptide core of the mucin becomes exposed such that epitopes within the core (not accessible within normal tissue-derived MUC1) may serve as potential antigens.

Clinical approaches targeting STn have thus far consisted solely of STn vaccines. The most advanced clinical candidate is Theratope, a therapeutic vaccine consisting of STn coupled to keyhole limpet hemocyanin. In in vivo mouse studies Theratope immunization induced a potent antibody response that was shown to mediate a delay in the growth of injected STn-expressing mammary carcinoma cells (Julien et al., British journal of cancer, 2009, 100, 1746-1751). However, Theratope failed to meet its primary endpoint in a phase III clinical trial in metastatic breast cancer. A leading hypothesis for why the Theratope trial missed its primary endpoint is that the patient population was not evaluated for STn expression prior to enrollment. Since STn expression in breast cancer is highly heterogeneous between patients, ranging from 25%-80% depending on the study and detection method, lack of ability to correlate STn expression with response may have masked any benefit from Theratope. Importantly, a subset of patients receiving hormonal therapy showed a significant 7.5 month increase in median overall survival when treated with Theratope compared to hormone therapy alone (Ibrahim et al., Journal of clinical oncology: official journal of the American Society of Clinical Oncology, 2004, 22, 2547; and Miles et al., The oncologist, 2011, 16, 1092-1100), validating the therapeutic potential of targeting STn in particular patient populations. Additionally, since the immune response often vanes considerably between vaccinated patients, vaccine approaches lack the ability to control or modulate antibody titer, resulting in wide ranges of therapeutic antibody exposure among patients. Nonetheless, Theratope was well tolerated with minimal toxicity, demonstrating the safety of targeting STn for cancer therapy.

The growing understanding of the molecular basis of STn expression in cancer cells strongly suggests that cells that express STn on any cell surface protein will also express STn on many (if not all) other O-glycosylated cell surface proteins, rendering it an excellent widely-distributed cancer-associated therapeutic target. Thus, STn positive cancer cell populations may be enriched for CSCs. In addition, recent data demonstrate that abrogation of STn expression renders cancers less aggressive with significant reductions in metastatic behavior (Gill et al., Proceedings of the National Academy of Sciences of the United States of America 2013, 110, E3152-3161).

### Colorectal Cancer

Colorectal cancer (CRC) has the 4^{th} largest incidence, and is currently the third leading cause of cancer-related death in the US. Currently, 20% of patients are diagnosed with metastatic disease and roughly 50% of patients with CRC will eventually develop metastases. For those diagnosed with metastatic disease, the 5-year survival rate is 13.1%. In patients with metastatic colon cancer (mCRC), there is precedence for use of therapeutic antibodies (e.g., monoclonal antibodies), such as anti-epidermal growth factor receptor (EGFR) monoclonal antibodies and anti-VEGF monoclonal antibodies.

In some embodiments, glycan-interacting antibodies of the present disclosure may be used to treat CRC and/or mCRC. In some cases, such glycan-interacting antibodies are anti-STn antibodies, including, but not limited to any of those described herein. Glycan-interacting antibodies used to treat CRC and/or mCRC may be conjugated with a cytotoxic agent (e.g., MMAE and MMAF). Glycan-interacting antibodies may be used in combination with other therapies such as therapies with a chemotherapeutic agent (e.g., fluoropyrimidine, oxaliplatin, and/or irinotecan) and/or with a therapeutic antibody (e.g., bevacizumab and/or anti-EGFR).

According to some embodiments, glycan-interacting antibodies used to treat colorectal cancer may be administered at a dose of from about 0.5 mg/kg to about 20 mg/kg. For example, antibodies may be administered at doses of from about 0.5 mg/kg to about 2 mg/kg, from about 1 mg/kg to about 5 mg/kg, from about 2.5 mg/kg to about 10 mg/kg, or from about 5 mg/kg to about 20 mg/kg.

### Combined Cancer Therapies

In some embodiments, compounds and compositions of the invention may be combined with one or more additional forms of cancer treatment. In some cases, such additional forms may include chemotherapeutic treatments. Accordingly, some methods of the invention include methods of treating cancer by administering at least one chemotherapeutic agent to a subject having cancer and administering a glycan-interacting antibody. Such antibodies may include anti-STn antibodies described herein.

As used herein, the term, “chemotherapy” refers to a form of treatment using chemical substances. Such chemical substances are referred to herein as “chemotherapeutic agents.” In the treatment of cancer, chemotherapeutic agents are agents that slow or prohibit the proliferation of cancer cells.

In some embodiments, chemotherapeutic agents of the invention may be nucleic acid antagonistic agents. Such agents primarily affect proliferating cells, such as cancer cells, and typically function by disrupting DNA repair and/or synthesis. In some cases, nucleic acid antagonistic agents are alkylating agents (e.g., bifunctional alkylators or monofunctional alkylators). Alkylating agents are reactive compounds that may be used to disrupt DNA synthesis in dividing cells. Alkylating agents of the invention may include, but are not limited to, cyclophosphamide, mechlorethamine, chlorambucil, melphalan, decarbazine, nitrosoureas, and temozolomide.

In other embodiments, nucleic acid antagonistic agents of the invention may include anthracyclines. Anthracyclines are bacterial derived compounds that disrupt nucleic acid synthesis. Anthracyclines of the invention may include, but are not limited to daunorubicin, doxorubicin, epirubicin, idarubicin, mitoxantrone, and valrubicin. In some embodiments, anthracyclines may be liposomally encapsulated.

In further embodiments, nucleic acid antagonistic agents may be histone deacetylase inhibitors and/or topoisomerase inhibitors. These inhibitors prevent changes in DNA supercoiling that are necessary for DNA synthesis and repair. Inhibitors of topoisomerase I may include, but are not limited to irinotecan and topotecan. Inhibitors of topoisomerase II may include, but are not limited to etoposide, teniposide, and tafluposide. Histone deacetylase inhibitors may include, but are not limited to vorinostat and romidepsin.

In some embodiments, nucleic acid antagonistic agents of the invention may include nucleotide analogs and/or nucleotide precursor analogs. Proliferating cells require nucleotides for incorporation into nucleic acids in resulting daughter cells. Nucleotide analogs may disrupt the formation of such nucleic acids or render them non-functional. Nucleotide analogs of the invention may include, but are not limited to azacitidine, azathioprine, capecitabine, doxifluridine, fluorouracil, gemcitabine, hydroxyurea, mercaptopurine, methotrexate, and tioguanine. In some embodiments, leucovorin as administered along with nucleotide analogs to enhance their effects and/or reduce harmful side effects.

In some embodiments, nucleic acid antagonistic agents of the invention are platinum-based agents. These agents disrupt nucleic acids by cross-linking them. Platinum-based agents of the invention may include, but are not limited to oxaliplatin, cisplatin, and carboplatin.

In some cases, chemotherapeutic agents of the invention include cytoskeletal disrupting agents. Actively dividing cells undergo major cytoskeletal changes that may be disrupted by these compounds. Cytoskeletal disrupting agents of the invention may include, but are not limited to vinca alkaloids, epothilones, paclitaxel, ABRAXANE® (paclitaxel protein-bound particles for injectable suspension), and docetaxel.

Although effective at targeting proliferating cancer cells, chemotherapeutic agents often affect some non-cancerous cells as well. Because of this, their administration is typically limited by dose, length of treatment, or area of treatment. Further, because chemotherapeutic agents primarily affect proliferating cells, non-proliferating cancer stem cells may remain viable after treatment and capable of reforming cancerous cells. Accordingly, in some embodiments, methods of the disclosure include methods of treating cancer in which at least one chemotherapeutic agent is first administered to a subject having cancer, followed by administration of a glycan-interacting antibody. In some cases, the glycan-interacting antibody is selected to target a specific cell surface glycan associated with chemotherapy-resistant cells. As used herein, the term “chemotherapy-resistant” is used to refer to cells that are unaffected by or that have limited susceptibility to chemotherapy treatment.

Methods of targeting chemotherapy-resistant cells (e.g., chemotherapy-resistant cancer stem cells) may take advantage of changes in STn expression in these cells occurring after chemotherapy treatment. In some cases, chemotherapy-resistant cells express STn before and/or after chemotherapy treatment. In some cases, cell surface STn expression in chemoresistant cells may be increased following chemotherapy treatment [e.g., due to altered expression of factors involved in STn synthesis (e.g., STnGalNAc I, T-synthase, or Cosme), decreased degradation, or other mechanisms leading to increased cell surface STn expression]. After chemotherapy treatments, some chemotherapy-resistant cells expressing cell surface STn may proliferate resulting in a population of STn-expressing tumor cells that are chemotherapy-resistant. In some embodiments, anti-STn antibodies may be used to target chemotherapy-resistant cells. In some cases, these cells are cancer stem cells. Accordingly, methods of the invention may include methods of administering an anti-STn antibody to target STn-expressing chemotherapy-resistant cells present after administration of one or more chemotherapeutic agent.

The identification of cell surface glycans on chemotherapy-resistant cells may be carried out by analyzing chemotherapy-resistant cells after chemotherapy treatment for the identity of cell surface glycans that distinguish these cells from surrounding cells. In some embodiments, such cell surface glycans may include, but are not limited to mucin-related antigens (including, but not limited to Tn, STn and Thomsen-Friedenreich antigen), blood group Lewis related antigens [including, but not limited to Lewis^{Y} (Le^{Y}), Lewis^{X} (Le^{X}), Sialyl Lewis^{X} (SLe^{X}) and Sialyl Lewis^{A} (SLe^{A})], glycosphingolipid-related antigens [including, but not limited to Globo H, stage-specific embryonic antigen-3 (SSEA-3) and glycosphingolipids having sialic acid], ganglioside-related antigens [including, but not limited to gangliosides GD2, GD3, GM2, fucosyl GM1 and Neu5GcGM3] and polysialic acid-related antigens. Many of such antigens are described in International Publication No. WO2015054600, the contents of which are herein incorporated by reference in their entirety. Analyses carried out to identify cell surface glycans expressed on cancer stem cells remaining after chemotherapy may be carried out according to any methods known in the art. In some cases, such analyses are carried out by obtaining a tissue sample and assessing the expression of cell surface glycans in the tissue sample using one or more immunological assay (e.g., immunohistochemical analysis, ELISA analysis, flow cytometric analysis, antibody array, or mass spectrometry).

In some embodiments, chemotherapy-resistant cells are analyzed to assess the expression level of cell surface STn. This may be carried out by obtaining a tissue sample and analyzing the sample for expression of cell surface STn [for example, using one or more immunological assay (e.g., immunohistochemical analysis, ELISA analysis, flow cytometric analysis, antibody array, or mass spectrometry)]. Where chemotherapy-resistant cells express STn, anti-STn antibodies may be administered to a subject after administration of chemotherapeutic agents.

In some embodiments, one or more tumors are primed for treatment with one or more glycan-interacting antibodies by contacting the tumors with at least one chemotherapeutic agent. According to such embodiments, priming a tumor for glycan-interacting antibody treatment refers to reducing proliferating cells in a tumor, leaving one or more chemotherapy-resistant tumor cells behind. According to such methods, glycan-interacting antibodies may be used to further reduce tumor volumes by eliminating chemotherapy-resistant cells that remain after treatment with one or more chemotherapeutic agents.

Administration of glycan-interacting antibodies after administration of one or more chemotherapeutic agent may be carried out from about 1 day to about one year after treatment with one or more chemotherapeutic agents (e.g., from about 1 day to about 10 days, from 1 week to about 4 weeks, from about 2 weeks to about 10 weeks, from about 1 month to about 3 months, from about 2 months to about 6 months, or from about 3 months to about 12 months). In some cases, administration of glycan-interacting antibodies may be carried out at least 1 year after treatment with one or more chemotherapeutic agents.

In some embodiments, multiple rounds of administration with one or more chemotherapeutic agents may be followed by administration of glycan-interacting antibodies (e.g., 2 rounds, 3 rounds, 4 rounds, 5 rounds, 6 rounds, 7 rounds, 8 rounds, 9 rounds, 10 rounds, or at least 10 rounds). In some cases, rounds of treatment are repeated until tissue analyses reveal that cancerous cells and/or chemotherapy-resistant cells are reduced or eliminated.

The dose of chemotherapeutic agents may be adjusted based on the size of the subject receiving treatment. In some embodiments, doses include those described by Calvo et al. 2014 (Calvo, E. et al., 2014. Chemotherapeutic agents and their uses, dosages, and toxicities. Cancer Network. p 1-12). In some cases, doses are adjusted based on the surface area of the subject being treated [typically measured in square meters (m²)]. Chemotherapeutic agents of the invention may be administered at doses of from about 0.01 mg/m² to about 1 mg/m², from about 0.1 mg/m² to about 5 mg/m², from about 1 mg/m² to about 20 mg/m², from about 10 mg/m² to about 100 mg/m², from about 50 mg/m² to about 500 mg/m², from about 200 mg/m² to about 2000 mg/m², or from about 1000 mg/m² to about 10000 mg/m². In some cases, chemotherapeutic agents of the invention are administered at a dose of at least 10000 mg/m². According to some methods, chemotherapeutic agents are administered intravenously.

In some embodiments, administration of chemotherapeutic agents includes administration of carboplatin. According to some methods, carboplatin is administered at a dose of from about 200 mg/m² to about 400 mg/m². In some embodiments, administration of chemotherapeutic agents includes administration of paclitaxel. According to some methods, paclitaxel is administered at a dose of from about 20 mg/m² to about 300 mg/m².

In some embodiments, glycan-interacting antibodies of the present disclosure are administered in combination with anti-angiogenic therapies (e.g., bevacizumab). According to some embodiments, methods of treating cancer are provided that include identifying a subject in need of cancer treatment, wherein the subject has cancer that is not fully responsive to treatment with at least one poly-ADP-ribose polymerase inhibitor, and administering an anti-STn antibody to the subject. Such anti-STn antibodies may include any of those known in the art or described herein.

In some embodiments, glycan-interacting antibodies of the present disclosure may be administered in combination with one or more immune checkpoint inhibitors. Immune checkpoints are molecules that regulate immune response. Such molecules may produce inhibitory or stimulatory signals to modulate the activity of an immune cell. In some cases, the immune checkpoints are inhibitory receptors expressed on the T-cell surface, such as cytotoxic T-lymphocyte-associated protein 4 (CTLA-4), programmed cell death protein 1 (PD1), and Lymphocyte-activation gene 3 (LAG-3). Inhibitors that prevent binding of such receptors to their ligands may help the body recognize and attack cancer cells. The immune checkpoint inhibitors may include, but are not limited to, CTLA-4 inhibitors (e.g., Ipilimumab and Tremelimumab), PD-1 inhibitors (e.g., Novolumab and Pidilizumab), programmed death-ligand 1 (PD-L1) inhibitors (e.g., Durvalumab and Atezolimab) and LAG-3 inhibitors (e.g., BMS-986016 and GSK2831781). In some cases, use of anti-STn antibodies may sensitize the immune system by killing the MDSCs and may increase the efficacy of checkpoint inhibitor treatment.

### Immune-Related Targets

In some embodiments, glycan-interacting antibodies of the invention may be immunomodulatory antibodies. As used herein, an immunomodulatory antibody is an antibody that enhances or suppresses one or more immune function or pathway.

Many bacterial glycans are known to include sialic acid. In some cases, such glycans allow bacteria to evade the innate immune system of hosts, including, but not limited to humans. In one example, bacterial glycans inhibit alternate complement pathway activation through factor H recognition. In another example, bacterial glycans mask underlying residues that may be antigenic. Some bacterial glycans participate in cell signaling events through activation of inhibitory sialic acid binding Ig-like lectins (Siglecs) that dampen the immune response to entities including certain sialylated moieties (Chen, X. et al., Advances in the biology and chemistry of sialic acids. ACS Chem Biol. 2010 Feb. 19; 5(2):163-76). In some embodiments, glycan-interacting antibodies of the present invention may be used to treat immune complications related to bacterial glycans.

Due to the foreign nature of Neu5Gc as described herein, some Neu5Gc glycans are immunogenic resulting in immune related destruction of cells and other entities where these glycans may be expressed. Such autoimmune destruction may be pathogenic. In some embodiments, glycan-interacting antibodies may be used to treat patients suffering from autoimmune disorders related to Neu5Gc glycans.

In some embodiments, immunomodulatory antibodies of the invention may be used to promote or suppress T cell-mediated immunity. Such antibodies may interact with one or more glycans present on T cells, T cell-related proteins and/or on one or more other cell types that interact with T cells. Immunomodulatory antibodies that enhance T cell mediated immunity may be used to stimulate T cell mediated targeting of cancer cells.

In some tumors, infiltration by tumor-associated macrophages (TAMs) may lead to immunosuppression promoting tumor cell viability and growth. This is thought to be due to immunosuppressive cell signaling that occurs through interactions between myeloid C-type lectin receptors (CLRs) present on TAMs and tumor-associated mucins (Allavena, P. et al., Clin Dev Immunol. 2010; 2010:547179). In some embodiments, binding of immunomodulatory antibodies of the invention to one or more tumor-associated mucin or TACA prevents immunosuppressive cell signaling in TAMs.

### Veterinary Applications

It is contemplated that glycan-interacting antibodies of the invention will find utility in the area of veterinary care including the care and treatment of non-human vertebrates. As described herein, the term “non-human vertebrate” includes all vertebrates with the exception of Homo sapiens, including wild and domesticated species such as companion animals and livestock. Non-human vertebrates include mammals, such as alpaca, banteng, bison, camel, cat, cattle, deer, dog, donkey, gayal, goat, guinea pig, horse, llama, mule, pig, rabbit, reindeer, sheep water buffalo, and yak. Livestock includes domesticated animals raised in an agricultural setting to produce materials such as food, labor, and derived products such as fiber and chemicals. Generally, livestock includes all mammals, avians and fish having potential agricultural significance. In particular, four-legged slaughter animals include steers, heifers, cows, calves, bulls, cattle, swine and sheep.

### Diagnostics

In some embodiments, compounds and compositions of the invention may be used as diagnostics. In some cases, antibodies of the invention may be used to identify, label or stain cells, tissues, organs, etc. expressing target antigens. In further embodiments, antibodies of the invention may be used to identify STn present in tissue sections (i.e., histological tissue sections), including tissue known or suspected of having cancerous cells. Such methods of using antibodies of the invention may in some cases be used to identify cancerous cells or tumors in tissue sections. Tissue sections may be from any tissue or organ including, but not limited to breast, colon, pancreas, ovary, endometrium, brain, liver, kidney, spleen, lung, skin, gastric system, stomach, intestine, esophagus, or bone.

In some embodiments, diagnostic methods of the invention may include the analysis of one or more cells or tissues using immunohistochemical techniques. Such methods may include the use of one or more of any of the glycan-interacting antibodies described herein. Immunohistochemical methods of the invention may include staining tissue sections to determine the presence and/or level of one or more glycosylated proteins or other markers. Tissue sections may be derived from subject tumors (e.g., patient tumors and animal tumors such as animal model tumors). Tissue sections may come from formalin-fixed or unfixed fresh frozen tissues. In some case, tissue section come from formalin fixed paraffin-embedded (FFPE) tissues. Glycan-interacting antibodies described herein may be used as primary antibodies. Primary antibodies are used to contact tissue sections directly and bind to target epitopes. Primary antibodies may be directly conjugated with a detectable label or may be detected through the use of a detection agent such as a secondary antibody. In some embodiments, primary antibodies or detection agents include an enzyme that can be used to react with a substrate to generate a visible product (e.g., precipitate). Such enzymes may include, but are not limited to horse radish peroxidase, alkaline phosphatase, beta-galactosidase, and catalase.

Anti-STn antibodies described herein may be used according to immunohistochemical methods of the present disclosure to detect STn-glycosylated proteins in tissues or cells. In some cases, these antibodies are used to detect and/or determine the level of STn in tumor tissues. Such tumor tissues may include tumor tissues included in tumor microarrays. Suitable tumor types include, but are not limited to breast, colon, ovarian, pancreatic, skin, intestinal, lung, endometrial, gastric, bladder, kidney, and brain tumors. Levels of anti-STn antibodies used in immunohistochemical staining techniques may be varied to increase visible staining or to decrease background levels of staining. In some embodiments, antibody concentrations of from about 0.01 μg/ml to about 50 μg/ml are used. For example, antibody concentrations of from about 0.01 μg/ml to about 1 μg/ml, from about 0.05 μg/ml to about 5 μg/ml, from about 0.1 μg/ml to about 3 μg/ml, from about 1 μg/ml to about 10 μg/ml, from about 2 μg/ml to about 20 μg/ml, from about 3 μg/ml to about 25 μg/ml, from about 4 μg/ml to about 30 μg/ml, or from about 5 μg/ml to about 50 μg/ml may be used.

In some embodiments, antibodies of the invention may be used to identify STn present on circulating MDSCs. Biological fluids carrying these cells may include, but are not limited to, blood, plasma, lymph, saliva, interstitial fluid, or any other fluid that contains cells. Such methods of using antibodies of the invention may in some cases be used to distinguish MDSCs from other circulating cells.

In some embodiments, diagnostic methods of the invention include methods of generating an STn-linked glycoprotein profile. As used herein the term “STn-linked glycoprotein profile” refers to a set of information indicating the level and/or identity of STn-linked glycoproteins in a sample or subject. Methods of generating an STn-linked glycoprotein profile may be carried out on a sample obtained from a subject. Such samples may be biological samples including, but not limited to, any of those described herein. Biological samples may be cellular samples. In some cases, cellular samples may include at least one tumor cell. In some embodiments, tumor cell samples may include BRCA1 mutant or non-BRCA1 mutant tumor cells.

Glycoproteins included in STn-linked glycoprotein profiles may include, but are not limited to, cancer cell markers, stem cell markers, cancer stem cell markers, and stem cell-related proteins. In some embodiments, glycoproteins identified and/or quantitated as part of a STn-linked glycoprotein profile may include, but are not limited to CD44, CD133, CD117, integrin, Notch, and Hedgehog.

Levels and/or identities of STn-linked glycoproteins in STn-linked glycoprotein profiles may be determined according to any methods known in the art for identifying proteins and/or quantitating protein levels. In some embodiments, such methods may include, but are not limited to mass spectrometry, array analysis (e.g., antibody array or protein array), Western blotting, flow cytometry, immunoprecipitation, and ELISA. STn-linked glycoproteins may in some cases be immunoprecipitated from a sample prior to analysis. Such immunoprecipitation may be carried out using an anti-STn antibody. Anti-STn antibodies used for immunoprecipitation of STn-linked glycoproteins may include any of those known in the art or described herein. In some embodiments, STn-glycoproteins are immunoprecipitated from biological samples using an anti-STn antibody and then identified and/or quantitated using mass spectrometry.

In some embodiments, cancer treatments are informed by STn-linked glycoprotein profile information. Accordingly, the present disclosure provides methods of treating cancer that include obtaining a sample from a subject in need of cancer treatment, generating an STn-linked glycoprotein profile from the sample, selecting a glycan-interacting antibody that binds to an STn-glycosylated protein from the STn-linked glycoprotein profile, and administering the glycan-interacting antibody to the subject. Glycan-interacting antibodies administered according to such methods may include one or more CDRs or variable domains taught herein.

In some embodiments, methods of the present disclosure may be used as companion diagnostics. As used herein, the term “companion diagnostic” refers to an assay, the results of which aid in the diagnosis or treatment of subjects. Companion diagnostics may be useful for stratifying patient disease, disorder or condition severity levels, allowing for modulation of treatment regimen and dose to reduce costs, shorten the duration of clinical trial, increase safety and/or increase effectiveness. Companion diagnostics may be used to predict the development of a disease, disorder or condition and aid in the prescription of preventative therapies. Some companion diagnostics may be used to select subjects for one or more clinical trials. In some cases, companion diagnostic assays may go hand-in-hand with a specific treatment to facilitate treatment optimization.

In some embodiments, methods of the present disclosure may be useful as companion diagnostics for diseases, disorders and/or conditions related to cancer. Some companion diagnostics of the present invention may be useful for predicting and/or determining the severity of one or more forms of cancer. Some companion diagnostics of the present invention may be used to stratify subjects by risk of developing one or more forms of cancer. Some companion diagnostics of the present invention may be used to facilitate and expedite drug development for cancer therapeutics.

In some embodiments, companion diagnostics of the present invention may include identifying STn present on circulating MDSCs. Such companion diagnostics may be used to select subjects for treatment with anti-STn therapy. Biological fluids carrying MDSCs may include, but are not limited to, blood, plasma, lymph, saliva, interstitial fluid, or any other fluid that contains cells. In some cases, blood samples may be peripheral blood. Methods of such companion diagnostics may include evaluation of STn on a subpopulation of circulating MDSCs, such as granulocytic, monocytic, or immature MDSCs. In some cases, levels of STn present on circulating MDSCs may be used to predict the levels of STn on MDSCs in a tumor. In some cases, levels of STn present on circulating MDSCs may be used to predict the levels of STn on tumor cells.

Some methods of the present disclosure include methods of identifying a candidate for anti-STn antibody treatment by obtaining a sample from a subject; identifying one or more MDSC in the sample; and evaluating the one or more MDSC for STn expression using an anti-STn antibody (e.g., SIA01). The sample may include a tumor sample. In some embodiments, the sample is a fluid sample. Fluid samples may include, but are not limited to, blood, plasma, lymph, saliva, and interstitial fluid. The sample may be a peripheral blood sample. The sample may be a spleen sample. The one or more MDSCs evaluated may include one or more of granulocytic lineage MDSCs, monocytic lineage MDSCs, and immature lineage MDSCs.

MDSCs may be identified by using antibodies specific for one or more MDSC marker antigens. Murine MDSCs are phenotypically characterized based on dual expression of CD11b and Ly6 antigens. Human MDSCs express the myeloid markers CD11b and CD33. CD14+ and HLA-DR− (or low) MDSCs are defined primarily as the monocytic lineage subpopulation, while human granulocytic lineage MDSCs are primarily defined as being CD15+(Youn et al, J Immunol. 2008; 181(8):5791-802). Immature lineage MDSCs are negative for both CD14 and CD15. Identifying MDSCs may be carried out using one or more of anti-CD33 antibody, anti-HLA-DR antibody, anti-CD45 antibody, anti-CD11b antibody, anti-CD14 antibody, and anti-CD15 antibody. Flow cytometry may be used to detect, sort, and/or count antibody-bound cells.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1. SIA01 Antibody

SIA01 antibody was developed as described in International Application Number PCT/US2016/061427, the contents of which are herein incorporated by reference in their entirety. SIA01 is a humanized IgG antibody having the variable domains of SEQ ID NOs: 1 and 2.

In flow cytometry-based binding studies, SIA01 was screened over a concentration range of 0 to 300 nM, comparing binding to MDA-MB-231 cells with or without transfection-induced STn expression. MDA-MB-231 cells are human breast cancer cells. They are grown in Earle's Minimum Essential Medium supplemented with 10% fetal calf serum (FCS), 100 μg/ml penicillin, 100 UI/ml streptomycin and 45 μg/ml gentamycin. Binding was determined using an anti-human APC conjugated secondary antibody and only live cells were considered (based on propidium iodide negative gating). 5,000 events were collected per sample on average. Data were analyzed using FLOWJO® software (BD Biosciences, San Jose, CA) and resulting APC means and % APC were obtained. These data were log transformed then fit to a nonlinear regression model to obtain a dose response curve and EC₅₀ binding information. Human isotype IgG1 antibody was used as an isotype negative control. An anti-epidermal growth factor receptor antibody (LA22, EMD Millipore, Billerica, MA) was used as a positive control.

For bovine submaxillary mucin (BSM) ELISA analysis, SIA01 was screened over a concentration range of 0 to 100 nM on BSM-coated wells. A subset of wells were treated with mild periodate solution before antibody binding to remove the side chain on terminal sialic acid residues (destroying the STn antigen). Optical densities of periodate and non-periodate-treated wells were determined and log transformed then fit to a nonlinear regression model to obtain a dose response curve. Optical density values obtained from periodate-treated wells were subtracted from non-periodate treated wells to obtain a periodate-sensitive STn binding curve and corresponding EC₅₀ values.

Optimized glycan array analysis was utilized to test SIA01 affinity and specificity for multiple glycans in a single experiment. The glycan array included 71 chemically synthesized and well-defined glycans, most of which were Neu5Ac and Neu5Gc glycan pairs. Array slides were obtained commercially (Arraylt Corp, Sunnyvale, CA) and included the glycans listed in the Table 4.

TABLE 4
Array glycans

| Glycan | |
|---|---|
| ID No. | Glycan |
| 1 | Neu5,9Ac2α2,3Galβ1,4GlcNAcβO(CH2)2CH2NH2 |
| 2 | Neu5Gc9Acα2,3Galβ1,4GlcNAcβO(CH2)2CH2NH2 |
| 3 | Neu5,9Ac2α2,6Galβ1,4GlcNAcβO(CH2)2CH2NH2 |
| 4 | Neu5Gc9Acα2,6Galβ1,4GlcNAcβO(CH2)2CH2NH2 |
| 5 | Neu5Acα2,6GalNAcαO(CH2)2CH2NH2 |
| 6 | Neu5Gcα2,6GalNAcαO(CH2)2CH2NH2 |
| 7 | Neu5,9Ac2α2,3Galβ1,3GlcNAcβO(CH2)2CH2NH2 |
| 8 | Neu5Gc9Acα2,3Galβ1,3GlcNAcβO(CH2)2CH2NH2 |
| 9 | Neu5,9Ac2α2,3Galβ1,3GalNAcαO(CH2)2CH2NH2 |
| 10 | Neu5Gc9Acα2,3Galβ1,3GalNAcαO(CH2)2CH2NH2 |
| 11 | Neu5Acα2,3Galβ1,4GlcNAcβO(CH2)2CH2NH2 |
| 12 | Neu5Gcα2,3Galβ1,4GlcNAcβO(CH2)2CH2NH2 |
| 13 | Neu5Acα2,3Galβ1,3GlcNAcβO(CH2)2CH2NH2 |
| 14 | Neu5Gcα2,3Galβ1,3GlcNAcβO(CH2)2CH2NH2 |
| 15 | Neu5Acα2,3Galβ1,3GalNAcαO(CH2)2CH2NH2 |
| 16 | Neu5Gcα2,3Galβ1,3GalNAcαO(CH2)2CH2NH2 |
| 17 | Neu5Acα2,6Galβ1,4GlcNAcβO(CH2)2CH2NH2 |
| 18 | Neu5Gcα2,6Galβ1,4GlcNAcβO(CH2)2CH2NH2 |
| 19 | Neu5Acα2,6Galβ1,4GlcβO(CH2)2CH2NH2 |
| 20 | Neu5Gcα2,6Galβ1,4GlcβO(CH2)2CH2NH2 |
| 21 | Neu5Acα2,3Galβ1,4GlcβO(CH2)2CH2NH2 |
| 22 | Neu5Gcα2,3Galβ1,4GlcβO(CH2)2CH2NH2 |
| 23 | Neu5,9Ac2α2,6GalNAcαO(CH2)2CH2NH2 |
| 24 | Neu5Gc9Acα2,6GalNAcαO(CH2)2CH2NH2 |
| 25 | Neu5Acα2,3GalβO(CH2)2CH2NH2 |
| 26 | Neu5Gcα2,3GalβO(CH2)2CH2NH2 |
| 27 | Neu5Acα2,6GalβO(CH2)2CH2NH2 |
| 28 | Neu5Gcα2,6GalβO(CH2)2CH2NH2 |
| 29 | Neu5,9Ac2α2,3GalβO(CH2)2CH2NH2 |
| 30 | Neu5Gc9Acα2,3GalβO(CH2)2CH2NH2 |
| 31 | Neu5,9Ac2α2,6GalβO(CH2)2CH2NH2 |
| 32 | Neu5Gc9Acα2,6GalβO(CH2)2CH2NH2 |
| 33 | Neu5Acα2,3Galβ1,3GalNAcβO(CH2)2CH2NH2 |
| 34 | Neu5Gcα2,3Galβ1,3GalNAcβO(CH2)2CH2NH2 |
| 35 | Neu5,9Ac2α2,3Galβ1,3GalNAcβO(CH2)2CH2NH2 |
| 36 | Neu5Gc9Acα2,3Galβ1,3GalNAcβO(CH2)2CH2NH2 |
| 37 | Neu5,9Ac2α2,6Galβ1,4GlcβO(CH2)2CH2NH2 |
| 38 | Neu5Gc9Acα2,6Galβ1,4GlcβO(CH2)2CH2NH2 |
| 39 | Neu5,9Ac2α2,3Galβ1,4GlcβO(CH2)2CH2NH2 |
| 40 | Neu5Gc9Acα2,3Galβ1,4GlcβO(CH2)2CH2NH2 |
| 41 | Neu5Acα2,8Neu5Acα2,3Galβ1,4GlcβO(CH2)2CH2NH2 |
| 42 | Neu5Acα2,8Neu5Acα2,8Neu5Acα2,3Galβ1,4GlcβO(CH2)2CH2NH2 |
| 43 | Galβ1,4GlcβO(CH2)2CH2NH2 |
| 45 | Galβ1,4GlcNAcβO(CH2)2CH2NH2 |
| 47 | GalNAcαO(CH2)2CH2NH2 |
| 51 | Galβ1,3GalNAcβO(CH2)2CH2NH2 |
| 52 | Galβ1,3GlcNAcαO(CH2)2CH2NH2 |
| 53 | Galβ1,3GlcNAcβO(CH2)2CH2NH2 |
| 54 | Galβ1,4GlcNAc6SβO(CH2)2CH2NH2 |
| 55 | Neu5Acα2,3Galβ1,4(Fucα1,3)GlcNAcβO(CH2)2CH2NH2 |
| 56 | Neu5Gcα2,3Galβ1,4(Fucα1,3)GlcNAcβO(CH2)2CH2NH2 |
| 57 | Neu5Acα2,3Galβ1,4(Fucα1,3)GlcNAc6SβO(CH2)2CH2NH2 |
| 58 | Neu5Gcα2,3Galβ1,4(Fucα1,3)GlcNAc6SβO(CH2)2CH2NH2 |
| 59 | Galβ1,3GlcNAcβ1,3Galβ1,4GlcβO(CH2)2CH2NH2 |
| 60 | Neu5Acα2,3Galβ1,3GlcNAcβ1,3Galβ1,4GlcβO(CH2)2CH2NH2 |
| 61 | Neu5Gcα2,3Galβ1,3GlcNAcβ1,3Galβ1,4GlcβO(CH2)2CH2NH2 |
| 62 | Neu5Acα2,3Galβ1,4GlcNAc6SβO(CH2)2CH2NH2 |
| 63 | Neu5Gcα2,3Galβ1,4GlcNAc6SβO(CH2)2CH2NH2 |
| 64 | Neu5Acα2,8Neu5Acα2,3Galβ1,4GlcβO(CH2)3NHCOCH2(OCH2CH2)6NH2 |
| 65 | Neu5Acα2,8Neu5Acα2,8Neu5Acα2,3Galβ1,4GlcβO(CH2)3NHCOCH2(OCH2CH2)6NH2 |
| 66 | Neu5Acα2,6(Neu5Acα2,3)Galβ1,4GlcβO(CH2)2CH2NH2 |
| 67 | Neu5Acα2,6(Neu5Gcα2,3)Galβ1,4GlcβO(CH2)2CH2NH2 |
| 68 | Neu5Acα2,6(KDNα2,3)Galβ1,4GlcβO(CH2)2CH2NH2 |
| 69 | Neu5Gcα2,8Neu5Acα2,3Galβ1,4GlcβO(CH2)2CH2NH2 |
| 70 | KDNα2,8Neu5Acα2,3Galβ1,4GlcβO(CH2)2CH2NH2 |
| 71 | Neu5Acα2,8Kdnα2,6Galβ1,4GlcβO(CH2)2CH2NH2 |
| 72 | Neu5Acα2,8Neu5Gcα2,3Galβ1,4GlcβO(CH2)2CH2NH2 |
| 73 | Neu5Acα2,8Neu5Gcα2,6Galβ1,4GlcβO(CH2)2CH2NH2 |
| 74 | KDNα2,8Neu5Gcα2,3Galβ1,4GlcβO(CH2)2CH2NH2 |
| 75 | Neu5Gcα2,8Neu5Gcα2,3Galβ1,4GlcβO(CH2)2CH2NH2 |
| 76 | Neu5Acα2,8Neu5Acα2,6Galβ1,4GlcβO(CH2)2CH2NH2 |

300 ml of epoxy blocking buffer was prepared by combining 15 ml of 2 M Tris buffer (pH 8) with 0.9 ml of 16.6 M ethanolamine and 284.1 ml of distilled water. The solution was brought to a final pH of 9.0 with HCl. The solution was filtered using a 0.2 μM nitrocellulose membrane. The epoxy buffer solution as well as 1 L of distilled water was pre-warmed to 50° C. Glass slides were arranged in a slide holder and quickly submerged in a staining tub with the warmed epoxy blocking buffer. Slides were incubated in the epoxy blocking buffer for 1 hour at 50° C. with periodic shaking to deactivate epoxy binding sites. Next, slides were rinsed and blocked with PBS with 1% OVA at 25° C. for one hour. Serum samples with polyclonal antibodies (1:1000) or purified monoclonal antibodies (1 μg/mL), were diluted in PBS with 1% OVA and added to the glycan array for one hour at 25° C. After extensive washing, binding of antibodies was detected by incubating glycan microarray slides with Cy3-conjugated anti-mouse IgG (Jackson Immunoresearch, West Grove, PA) for one hour. Slides were then washed extensively, dried and scanned with a Genepix 4000B scanner (Laser at 100%; gain at 350; 10 μm pixels). Raw data from scanned images were extracted using the Genepix software and analysis of raw data was carried out. Antibodies were considered to be highly specific for AcSTn and GcSTn if they demonstrated binding to both molecules, but not to Tn or any other glycans on the array.

Based on array analysis, SIA01 was classified according to an array glycan binding profile. Antibodies classified as “Group 1” antibodies, capable of binding AcSTn and GcSTn, if they demonstrate binding to glycans 5, 6, 23 and 24. Such antibodies are referred to as pan-STn antibodies due to their ability to associate with a wider range of STn structures and the portion of STn indicated by the largest ellipse in FIG. 1A. Antibodies are classified as “Group 2” antibodies, capable of binding STn as well as some related structures that include an O-linkage to serine or threonine, if they bind to glycans 5, 6, 23, 24, 27 and 31. These antibodies are thought to associate with the portion of STn indicated by the largest ellipse in FIG. 1B. Some Group 2 antibodies preferably bind to structures with AcSTn over structures with GcSTn. Antibodies are classified as “Group 3” antibodies (capable of binding STn, but may also bind a broader set of related structures) if they bind glycans 5, 6, 23, 24, 17, 3, 19, 37, 27 and 31. Unlike Group 2 antibodies, Group 3 antibodies do not require that such structures have an O-linkage to serine or threonine. Group 3 antibodies are thought to associate with the portion of STn indicated by the largest ellipse in FIG. 1C. Finally, antibodies are “Group 4” antibodies, capable of binding to both AcSTn and GcSTn as well as the un-sialylated Tn antigen (therefore having broader specificity) if they bind to glycans 5, 6, 23, 24 and 47. Group 4 antibodies are thought to associate with the portion of STn indicated by the largest ellipse in FIG. 1D.

Results from flow cytometry, ELISA, and glycan array analysis of SIA01 are presented in Table 5.

TABLE 5
Antibody characterization results

| MDA-MB-231-STn+ | BSM ELISA | Array glycan |
|---|---|---|
| cell binding [EC₅₀ (nM)] | [EC₅₀ (nM)] | binding profile |
| 2.0 | 4.2 | Group 1 |

SIA01 demonstrated binding to cell- and BSM-associated STn. No binding was observed with human IgG1 isotype control (Southern Biotech, Birmingham, AL). Further, glycan array analysis indicated that SIA01 has a “Group 1” binding profile.

### Example 2. STn Expression in Primary Patient Tumor Samples

Fluorescence Activated Cell Sorting (FACS) was used to detect tumor cells expressing STn and CD133 on their surface. Fresh tumor tissue was dissociated into single cells and the resulting samples were analyzed by FACS using each of the five panels provided in Table 6. The goal of these studies was four-fold: (1) to compare commercial anti-STn antibodies (B72.3 and CC49) to SIA01 anti-STn antibody; (2) to identify stem-like or non-stem tumor cells bearing STn; (3) to identify lymphoid cells bearing STn; (4) to examine T cells (CD4, CD8, CD25, CD69) present in tumors; and (5) to analyze MDSC subpopulations.

TABLE 6
Antibody panels

| Panel 1 | Panel 2 | Panel 3 | Panel 4 | Panel 5 |
|---|---|---|---|---|
| SIA01-PE | B72.3-PE | CC49-PE | CD25-APC | SIA01-PE |
| CD133 Clone | CD133 Clone | CD133 Clone | CD4- | CD33-APC |
| 450-FITC | 450-FITC | 450-FITC | APCVio770 | |
| CD 133 Clone | CD133 Clone | CD133 Clone | CD8-Pacific | HLA-DR-FITC |
| 293C3- | 293C3- | 293C3- | Blue | |
| PEVio770 | PEVio770 | PEVio770 | | |
| Anti-Tumor | Anti-Tumor | Anti-Tumor | CD45-VioGreen | CD11b-APC- |
| Marker-APC | Marker-APC | Marker-APC | | VIO770 |
| CD34-VioBlue | CD34-VioBlue | CD34-VioBlue | CD69-PE | CD14- VioBlue |
| CD45-VioGreen | CD45-VioGreen | CD45-VioGreen | 7AAD | CD15- PE- |
| | | | | VIO770 |
| 7AAD | 7AAD | 7AAD | | CD45-VioGreen |
| | | | | 7AAD |

Anti-CD133 antibodies, anti-CD25, anti-HLA-DR, anti-CD15, anti-CD33, anti-CD11b, anti-CD14, and anti-CD69 antibodies were purchased from Miltenyi Biotec, Cambridge, MA. Anti-CD4 and anti-CD8 antibodies were purchased from BD Biosciences, San Jose, CA. Anti-CA19-9 and anti-CA-125 antibodies were purchased from AbCam, Cambridge, UK. Anti-PSA antibody was purchased from EMD Millipore, Billerica, MA. Antibody CC49 is described in Muraro, R. et al., 1988. Cancer Res. 48: 4588-96. Antibody B72.3 is described in Colcher, D. et al., 1981. PNAS. 78(5): 3199-203. Both CC49 and B72.3 were purchased from Santa Cruz Biotechnology, Dallas, TX. PE is phycoerythrin, FITC is fluorescein, APC is allophycocyanin, and 7AAD is 7-aminoactinomycin D. Anti-tumor marker antibodies included anti-CD66 (CEA) for recognition of colon tumor cells, anti-PSA for recognition of prostate tumor cells, anti-CA19.9 for recognition of pancreatic tumor cells, and anti-CA-125 for recognition of ovarian tumor cells. Antibodies against CD4, CD8, CD69, and CD25 were used to identify immune cells. CD4 and CD8 were used to identify T cells. T cell activation subsets were identified with CD4+CD25+, CD4+CD69+, CD8+CD25+, and CD8+CD69+. Single cell mixtures prepared from primary human tumor samples contain tumor cells, lymphocytes, endothelial cells and stromal cells from tumor blood vasculatures, all samples were gated on live cells using 7AAD. Panels 1-3 were to study the solid tumor, CD34 was used to exclude endothelial cells while CD45 used to exclude lymphocytes. Cancer stem cells (CSCs) were defined as CD45− CD34− Tumor marker+CD133+ cells. Among CSCs and regular tumor cells defined as CD45− CD34− tumor marker+, STn positivity in each of these populations was also evaluated. Panel 4 was used to identify immune infiltrates into the tumor. This panel was gated on the CD45+ population to examine only the immune cells (CD25, CD4, CD8, CD69). In Panel 5, the different MDSC populations within the immune cell infiltrate were characterized. Antibodies against CD33, HLA-DR, CD11 b, CD14 and CD15 were used to identify myeloid-derived suppressor cells (MDSCs). Human MDSCs are typically CD45+, HLA-DR−, CD33+ and CD11b+. Using CD14 and CD15 markers, MDSCs can be further delineated into monocytic lineage (CD14+, CD15−), granulocytic lineage (CD14−, CD15+), or a more immature lineage (CD14−, CD15−).

Initial studies utilized a total of 10 samples derived from patients with colon cancer. A total of 10 samples were taken from either pancreatic, prostate or ovarian cancer. All 20 samples were analyzed for STn expression, comparing anti-STn antibody SIA01 to commercial antibodies B72.3 and CC49. Patients were de-identified and their samples assigned numbers. Patient history such as gender, diagnosis, age at time of diagnosis, age at time of collection, status of tumor, CA125/CA19-9/PSA or CA125 expression level (if applicable), BRCA1/2 status (if applicable), treatment history, collection protocol, specimen size and specimen ischemia time were recorded. For example: Samples 160080-2, 160093-2, 160101-2, 160102-2, and 160115-2 were derived from 5 patients with colon tumors, sample 160089-2 was derived from a pancreatic tumor, and sample 160104-2 was derived from a colon tumor formed in the liver due to metastasis. For analysis, cells were dissociated from patient-derived tumor tissues and combined with enzymes for mechanical digestion to break down tissue into single cell suspensions. Red blood cells were lysed and isolated away from tumor and immune cells. Cells were then split into 5 portions and labeled with antibody cocktail panels described above. Treated cells were then subjected to flow cytometry analysis and 10,000 events were acquired for each sample. A portion of the tissue was not digested and saved for immunohistochemistry, along with a serum sample isolated from patient's blood for comparative analyses of STn expression.

The percentage of cells from each sample that were CD133-positive and recognized by each of the three anti-STn antibodies was determined. All anti-STn antibodies recognized a similar percentage of CD133+ tumor cells from individual tumors, suggesting that a sub-population of cancer stem cells also express STn antigen. Samples 160080-2, 160093-2, 160101-2, 160102-2, and 160115-2 were derived from patient colon tumors, sample 160089-2 was derived from a pancreatic tumor, and sample 160104-2 was derived from a colon tumor formed in the liver due to metastasis. Examining these samples, B72.3 and CC49 staining was nearly identical with a significant r value (r=0.97 and p<0.001).

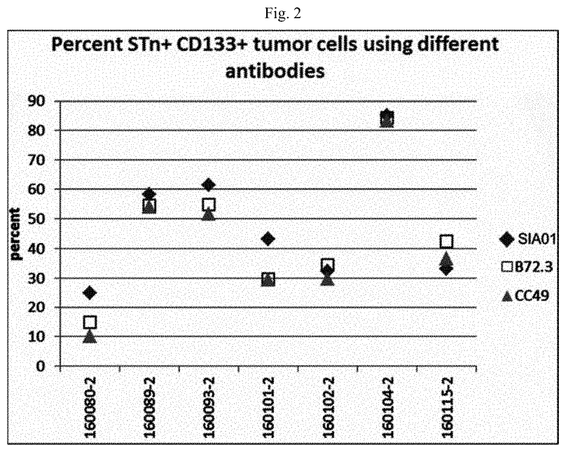

Each of the three STn antibodies indicated a similar, but not identical, percentage of CD133+/STn+ cells with SIA01 recognizing the highest percentage of these cells in the majority of samples (see FIG. 2).

MDSCs suppress antitumor immune responses by suppressing T cell responses. These cells typically express CD45 and CD133 cell markers. Binding of anti-STn antibodies to CD45+/CD133+ cells was used to determine if such antibodies may be used to target MDSCs for inhibition and/or elimination. The percentage of cells in each sample that were positive for all three markers are presented in Table 7.

TABLE 7
FACS analysis for STn expression on CD45+/CD133+ cells

| Sample | Tumor | % STn+/CD45+/CD133+ |
|---|---|---|
| 160080-2 | colon | 40.0 |
| 160089-2 | pancreatic | 70.0 |
| 160093-2 | colon | 45.1 |
| 160101-2 | colon | 73.4 |
| 160102-2 | colon | 67.8 |
| 160104-2 | colon met liver | 85.2 |
| 160115-2 | colon | 36.4 |

Sample 160104-2 demonstrated the highest percentage of cells with all three markers with samples 160101-2 and 160089-2 also demonstrating the presence of 70% or more of these cells. These results demonstrate the ability of SIA01 to target CD45+/CD133+ cells and demonstrate the relative abundance of STn+/CD45+/CD133+ cells in tumors tested. Further, the results indicate that anti-STn antibodies may be a strong candidate for targeting colon cancer cell-derived and pancreatic cancer cell-derived tumors.

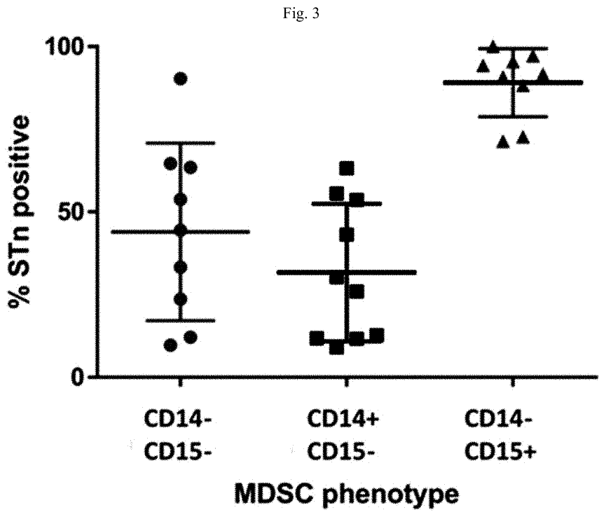

STn expression within the different subpopulations of MDSCs was also analyzed. MDSCs are a heterogeneous group of cells that can be divided into granulocytic (CD14−, CD15+), monocytic (CD14+, CD15−), and immature (CD14−, CD15−) cell lineages. Immune cells were sorted to identify MDSCs using the following marker surface expression profile: CD45+, HLA-DR−, CD33+ and CD11b+. The MDSCs were then sorted using anti-CD14 and anti-CD15 antibodies and STn expression within the different subpopulations was analyzed using SIA01 anti-STn antibody. Virtually all granulocytic MDSCs were positive for STn expression. In contrast, only 30˜50 percent (approximately) of the monocytic and immature MDSCs were positive for STn expression (FIG. 3). These findings support the use of anti-STn antibodies to target granulocytic MDSCs and/or distinguish these cells from other MDSC classes.

### Example 3. MDSC STn Expression in Colon Tumor Samples

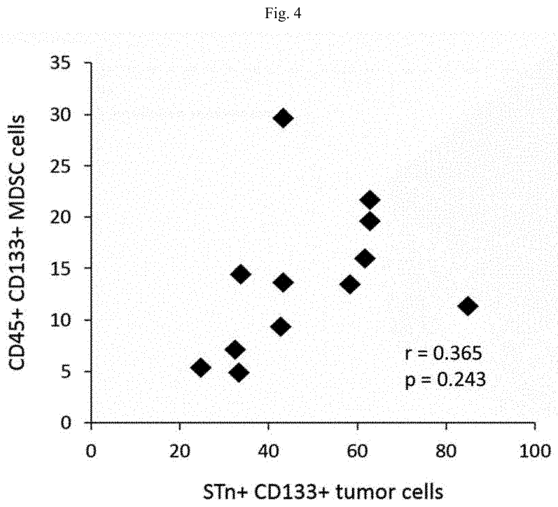

Five colon tumor samples were analyzed for STn+ expression in the tumor and MDSCs. The percentage of STn+/CD45+/CD133+ MDSCs is plotted against the percentage of STn+/CD133+ tumor cells (see FIG. 4). The result demonstrates an emerging correlation between tumor STn and MDSC STn expression levels.

### Example 4. MDSC STn Expression in Paired Blood Samples

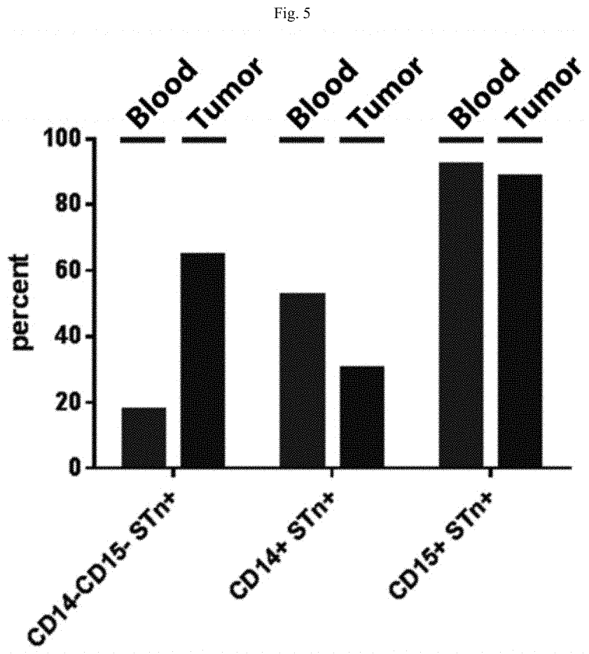

STn expression on MDSCs in a human bladder cancer tumor and matched blood sample was assessed. Both samples were analyzed for STn expression on granulocytic (CD14−, CD15+) MDSCs, monocytic (CD14+, CD15−) MDSCs, and immature (CD14−, CD15−) MDSCs (see FIG. 5). The data shows that STn expression in peripheral MDSCs is indicative of STn expression on MDSCs in the tumor. This finding suggests detecting MDSC STn expression in blood samples may be used as a companion diagnostic for anti-STn antibody treatment.

### Example 5. MDSC STn Expression Across Tumor Types

To evaluate the relationship between tumor STn expression and MDSC STn expression, human tumors of the following types were analyzed: bladder, colon, metastatic colon (colon met), endometrial, gastric, kidney, metastatic kidney (kidney met), ovarian, and pancreatic. Fresh tumors were digested and assessed for both MDSC STn expression and tumor STn expression according to the methods described in Example 2, using antibody panels 1 and 5 with or without replacement of SIA01-PE with isotype control to normalize for non-specific IgG binding. The relationship between STn+ MDSC (mMDSCs and PMN-MDSCs) and tumor type as well as tumor STn expression status was characterized.

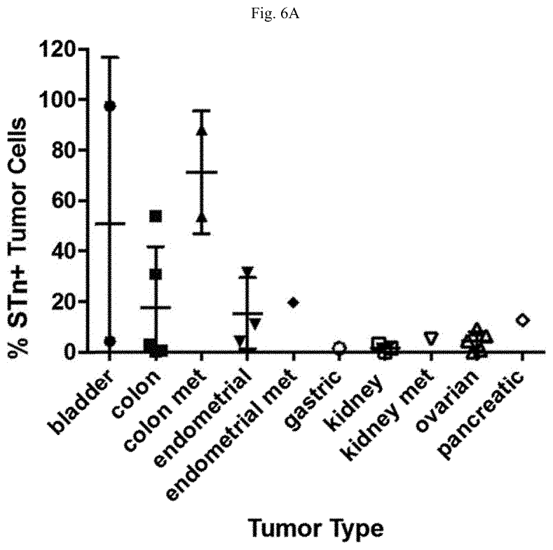

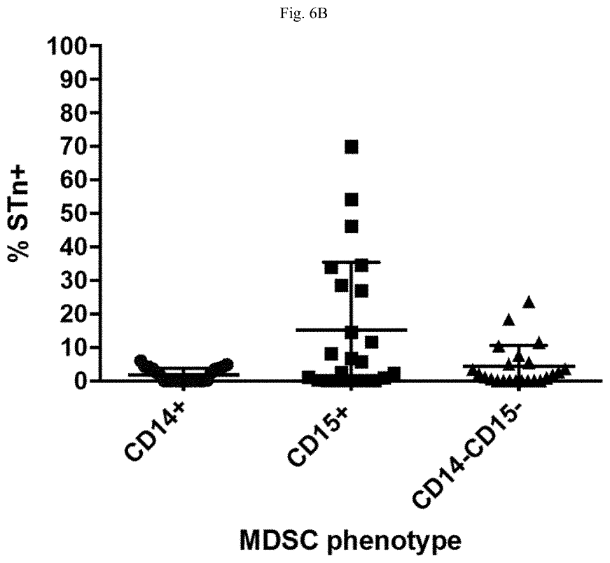

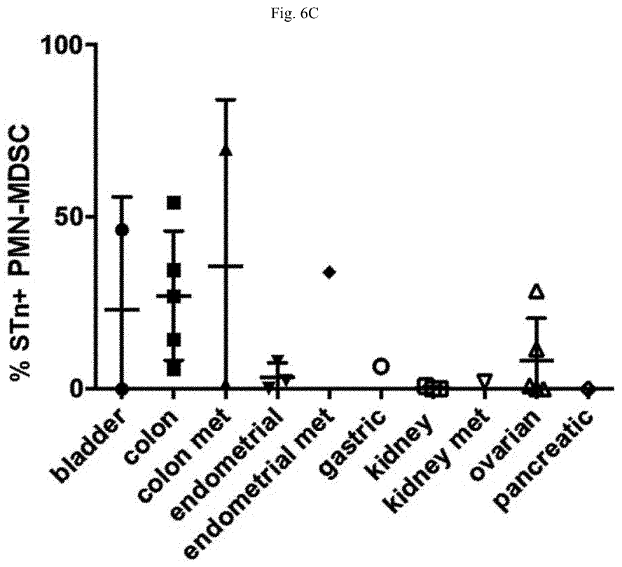

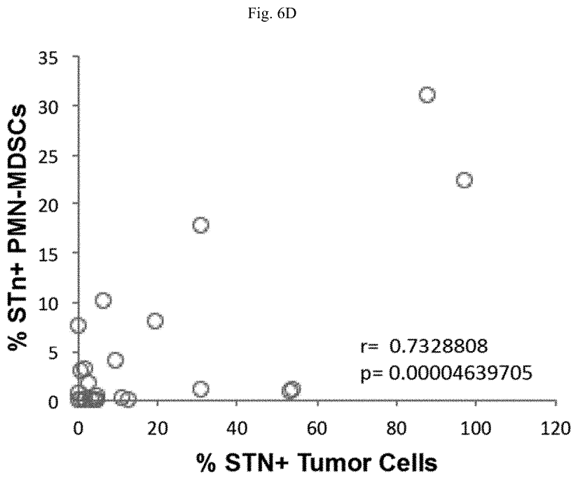

The percentage of STn positive (STn+) tumor cells in the samples studied is presented in FIG. 6A. Bladder, colon, and endometrial tumors exhibited the highest percentage of STn+ cells of those tested. Of the MDSCs, PMN-MDSCs (CD15+) had the highest percentage of cells expressing STn (see FIG. 6B). Among the tumor tissues tested, STn+ PMN-MDSCs were most commonly found in bladder, colon, endometrial, and ovarian tissues (FIG. 6C). Correlation between STn expression in tumors and MDSCs was also observed, indicating that PMN-MDSC STn expression is related to tumor STn expression (FIG. 6D).

### Example 6. Evaluation of Tumor TMAs

STn expression was evaluated in a tissue microarray (TMA) containing a number of human and mouse tumors. Of the mouse tumors analyzed, only one melanoma showed some STn expression. This suggests STn expression in murine lines may be rare.

### Example 7. Evaluation of Syngeneic Mouse Models

Existing syngeneic mouse models are characterized for STn expression on tumor and MDSC through flow cytometry analysis. Samples of freshly harvested syngeneic tumors are analyzed that include 4T1 mouse mammary tumor, CT26 mouse colon carcinoma, Lewis Lung carcinoma, UNKC-6141 pancreatic tumor and Renca kidney tumor. The relationship between tumor and MDSC STn expression in these murine models is examined and utilized to inform model selection for in vivo studies.

### Example 8. Generation of Xenograft Mouse Models

A xenograft mouse model is generated using an engineered cell line expressing STn. A STn-murine tumor cell line (e.g., 4T1, CT26, or EL4 murine breast cancer line) is transduced with an expression vector for ST6GalNAc I (SBH Sciences, Natick, MA) to create stable cell lines expressing STn. 4T1 has been shown to be STn-in vitro, and has a high percentage of MDSC infiltrates when grown in vivo. The level of MDSC infiltration in CT26 and EL4 tumors is typically even higher. Transfected clones are characterized by gene expression (qPCR) and flow cytometry via binding to the anti-STn antibodies. One high expressing clone and one wild-type line are implanted in immune-competent mice and STn expression on MDSCs are quantified in both. The relationship of MDSC STn expression and tumor STn expression is then assessed. A mouse model with desired properties is selected for in vivo studies.

### Example 9. Characterization of Mouse Anti-STn Antibody-Drug Conjugate (ADC)

A mouse anti-STn antibody-drug conjugate (ADC) is generated by contacting a mouse anti-STn antibody with maleimidocaproyl-valine-citruline-p-aminobenzyloxycarbonyl-monomethyl auristatin E (MC-vc-PAB-MMAE, referred to herein as CL-MMAE). The resulting conjugation is maleimide-cysteine based, where the antibody interchain disulfide bonds are reduced with TCEP and then linked to the maleimide moiety of the drug.

The mouse anti-STn ADC is assessed for STn binding via glycan array analysis and tumor cytotoxicity via selectively killing of STn+ cell lines in vitro. As a control to toxin mediated cytotoxicity, the unconjugated antibody and an isotype IgG antibody are evaluated for their ability to achieve MDSC depletion through complement-mediated lysis or ADCC when injected into mice.

### Example 10. In Vivo Tumor Inhibition in Mice

Anti-STn ADC-dependent in vivo tumor inhibition is assessed in a syngeneic mouse model utilizing a mouse syngeneic tumor line identified above that contains STn+ MDSC or an engineered cell line expressing STn. The anti-STn ADC is evaluated for antibody-mediated killing of MDSC on growth of a syngeneic mouse tumor. Animals are treated with vehicle, unconjugated antibody, conjugated antibody (1 mg/kg, 2.5 mg/kg, 5 mg/kg), isotype control, and a chemotherapeutic control. Treatments are administered once per week for four weeks. Tumor growth is observed throughout the study. Remaining tumor at study termination is removed and characterized for tumor and MDSC STn expression and MDSC content. Depletion of STn+ MDSCs within the peripheral blood is also assessed.

### Example 11. In Vivo Models for Human Tumors

Upon successful completion of in vivo mouse studies, in vivo models are identified for targeting human MDSCs through STn. Successful identification of appropriate patient tumor models and subsequent demonstration of pre-clinical efficacy of the human anti-STn ADC provides the basis for clinical translation. Additionally, these anti-STn antibodies or ADCs are evaluated in combination therapy with immune checkpoint inhibitors (e.g., anti-PD-1 or anti-CTLA4) for antitumor activity. Reducing the inhibition of T cells through depletion of MDSCs provides a basis to enhance the efficacy of existing immuno-therapies.

### Example 12. Association Between Tumor and MDSC STn Expression

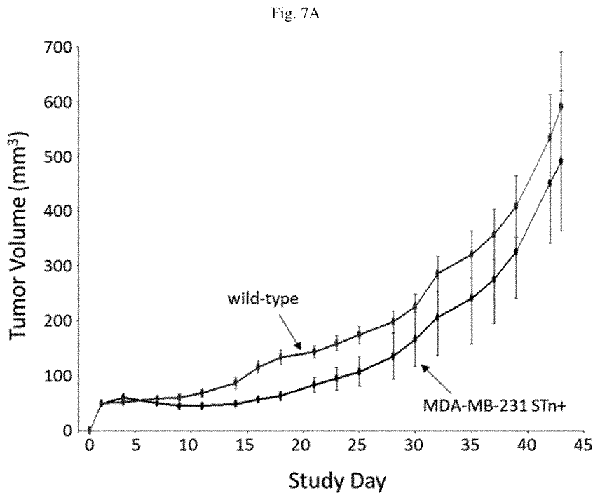

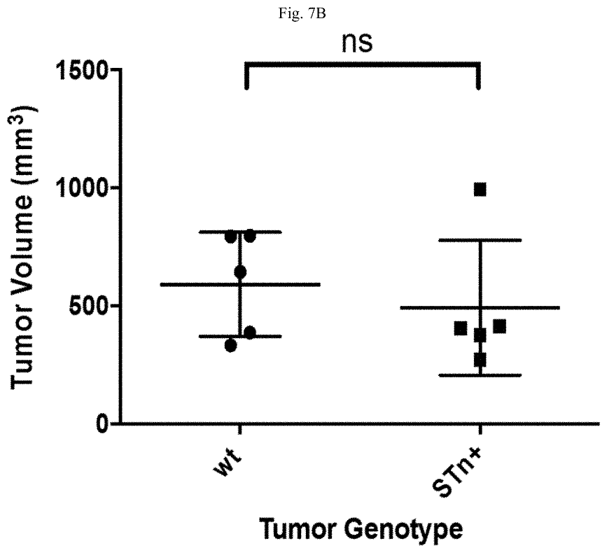

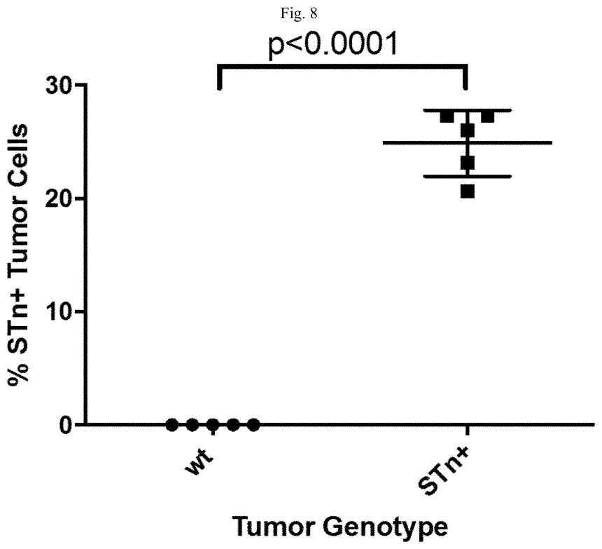

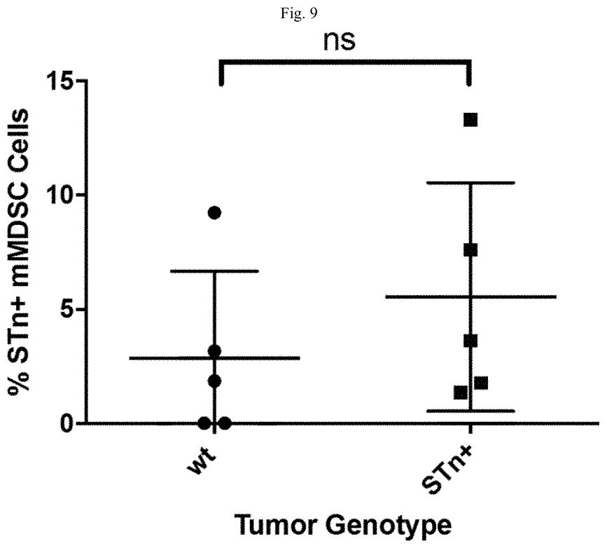

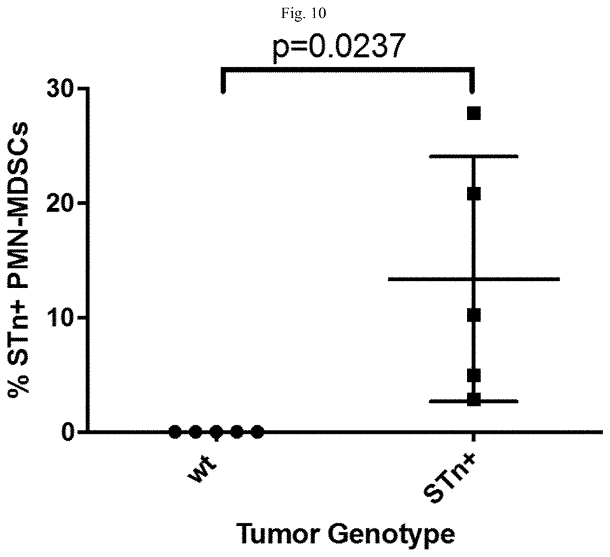

To explore the association between tumor and MDSC STn expression, MDA-MB-231 breast cancer cells, wild-type or engineered to express STn (MDA-MB-231 STn+ cells), were used to initiate xenograft tumors in immune-deficient mice. Average tumor volumes for each group were assessed over time (FIG. 7A) and at the end of the study (FIG. 7B). Terminal tumor tissues were collected and analyzed according to the methods described in Example 2 using antibody panels specific for murine MDSCs [anti-CD45 and anti-CD11b antibodies for MDSC identification and anti-Ly6c and anti-Ly6g antibodies for distinction between mMDSCs (Ly6c+/Ly6g−) and PMN-MDSCs (Ly6c−/Ly6g+)] and SIA01 (or isotype control) to assess STn expression among tumor and MDSC populations. As expected, STn expression was significantly higher in MDA-MB-231 STn+ tumors when compared to wild-type tumors (FIG. 8). Also, while low levels of STn were associated with mMDSCs from both tumor types (FIG. 9), STn expression by PMN-MDSCs was significantly higher in MDA-MB-231 STn+ tumors (FIG. 10). These results indicate that tumor STn expression correlates with PMN-MDSC STn expression and that SIA01 selectively binds to STn associated with these cells. Accordingly, SIA01 may be used to target STn+ MDSCs and reduce MDSC immune-suppressive activity.

### Example 13. Targeted Destruction of MDSCs in Tumor and Spleen Using Anti-STn Antibodies

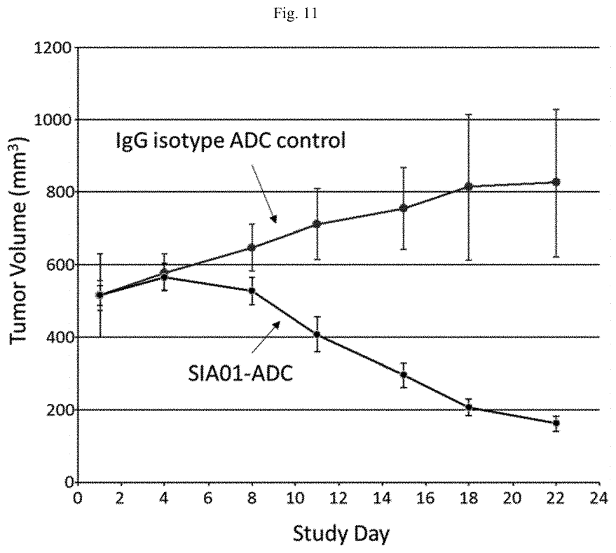

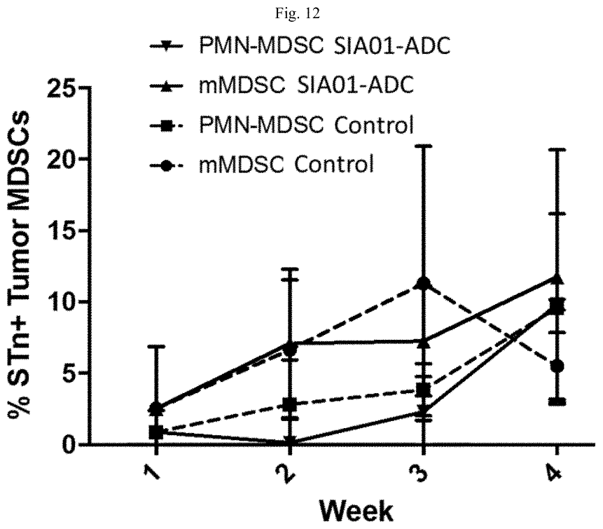

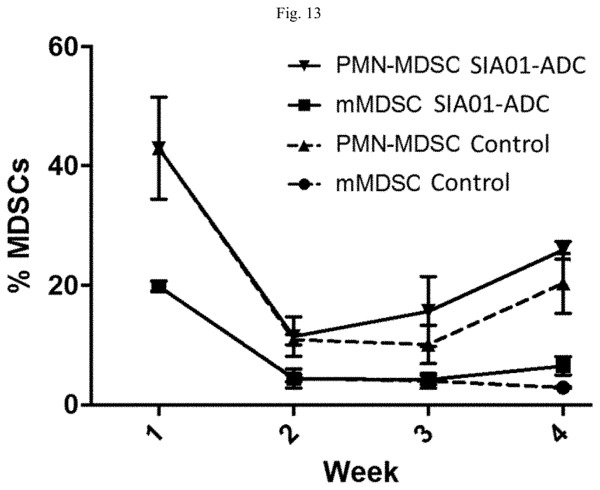

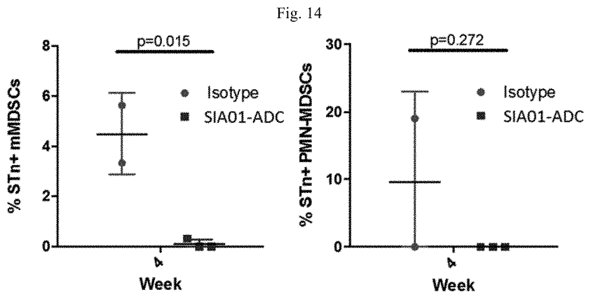

The ability of anti-STn antibodies to target and reduce MDSCs in mouse tumors and spleen was evaluated. OVCAR3 cells, known to express STn, were used to establish xenograft tumors in immune-deficient mice. After tumor volumes reached 500 mm³, mice were administered 5 mg/kg SIA01 conjugated with MMAE (SIA01-ADC) or an isotype control IgG-MMAE via weekly intravenous injection. As expected, tumor volumes were reduced with SIA01-ADC (FIG. 11). After 4 weeks, tumor and spleen cells were harvested and analyzed according to the methods described in Example 12. While no significant difference between treatment groups was observed for STn+ tumor MDSC levels (FIG. 12) or spleen MDSC levels (FIG. 13), a reduction in STn+ MDSCs in the spleen was observed (FIG. 14). This collectively suggests that the antibody may be depleting specifically STn+ MDSC in the spleen, potentially due to antibody dependent cellular cytotoxicity (ADCC). In the tumor environment, where direct cytotoxicity of the toxin may play a more prominent role, a different toxin conjugate optimized for terminally differentiated cells such as MDSC may be necessary. Accordingly, anti-STn antibodies are well suited to target STn+ subpopulations of MDSCs in the tumor and spleen. Depletion of STn+ MDSCs is likely to be achieved through ADCC or the use of a conjugated cytotoxic agent able to target cells at any phase of the cellular cycle (e.g., duocarmycin, gemcitabine, etc.).

1. A method of targeting one or more Myeloid-Derived Suppressor Cells (MDSCs) comprising contacting a subject or subject sample with a glycan-interacting antibody, wherein the subject or subject sample includes at least one MDSC.

2. The method of claim 1, wherein the glycan-interacting antibody binds to STn.

3-9. (canceled)

10. A method of treating a subject comprising administering an anti-STn antibody to the subject, wherein the subject has at least one tumor comprising an MDSC.

11-12. (canceled)

13. The method of claim 10, wherein the anti-STn antibody is an IgG antibody.

14. The method of claim 10, wherein the anti-STn antibody comprises an antibody-drug conjugate (ADC).

15. The method of claim 14, wherein the ADC comprises at least one conjugate selected from one or more of an auristatin, a maytansine, a tubulysin, a vinca alkaloid, a pyrrolobenzodiazepine dimer, a camptothecin, a duocarmycin, an amanitin, a phosphoinositide 3-kinase (PI3K) inhibitor, and a mitogen-activated protein kinase kinase (MEK) inhibitor.

16. The method of claim 15, wherein the ADC comprises one or more polymer, wherein the one or more polymer connects the anti-STn antibody and the at least one conjugate.

17. The method of claim 16, wherein the one or more polymer comprises one or more of a poly(ethylene glycol) (PEG), a poly(N-(2-hydroxypropyl)methacrylamide) (polyHPMA), a poly(α-amino acid), a carbohydrate polymer, a glycopolysaccharide, a glycolipid, a glycoconjugate, a polyglycerol, a polyvinyl alcohol, a poly(acrylic acid), a polyketal, and a polyacetal.

18. The method of claim 17, wherein the one or more polymer comprises a poly(1-hydroxymethylethylene hydroxymethylformal) (PHF).

19-23. (canceled)

24. A method of identifying a candidate for anti-STn antibody treatment, the method comprising:
a. obtaining a sample from a subject;
b. identifying one or more MDSC in the sample; and
c. evaluating the one or more MDSC for STn expression using an anti-STn antibody.

25. The method of claim 24, wherein the sample is a tumor sample.

26. The method of claim 24, wherein the sample is a fluid sample.

27. The method of claim 26, wherein the fluid sample is selected from blood, plasma, lymph, saliva, and interstitial fluid.

28-42. (canceled)


SEQUENCE LISTING (6 sequences)

SEQ ID NO: 1 - protein, 115 aa, synthetic construct
  REGION 1..115: note = Description of Artificial Sequence: Synthetic polypeptide
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 2 - protein, 113 aa, synthetic construct
  REGION 1..113: note = Description of Artificial Sequence: Synthetic polypeptide
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 3 - protein, 330 aa, Homo sapiens [sequence omitted]

SEQ ID NO: 4 - protein, 107 aa, Homo sapiens [sequence omitted]

SEQ ID NO: 5 - protein, 10 aa, Homo sapiens
EQKLISEEDL

SEQ ID NO: 6 - protein, 8 aa, synthetic construct
  REGION 1..8: note = Description of Artificial Sequence: Synthetic peptide
DYKDDDDK